(19) United States
(12) Patent Application Publication
(10) Pub. No.: US 2002/0157123 A1
(43) Pub. Date: Oct. 24, 2002

(54) Compositions and methods for treating enzyme deficiency

(76) Inventors: Arnold J. Reuser, Rotterdam (NL); Ans T. Van der Ploeg, Poortugaal (NL); Martin Ph. Verbeet, Amsterdam (NL)

Correspondence Address: NATH & ASSOCIATES, 1030 15th STREET, 6TH FLOOR, WASHINGTON, DC, 20005, US

(21) Appl. No.: 10/014,511

(22) Filed: Dec. 14, 2001

Related U.S. Application Data
(63) Continuation of application No. 09/770,496, filed on Jan. 29, 2001.
(60) Provisional application No. 60/001,796, filed on Aug. 2, 1995.
(60) Provisional application No. 60/111,291, filed on Dec. 7, 1998.

(51) Int. Cl. A01K 67/027

(52) U.S. Cl. 800/14; 800/18

(57) ABSTRACT

The invention provides transgenic nonhuman mammals producing phosphorylated lysosomal proteins in their milk, methods of generating the same, pharmaceutical compositions for use in enzyme replacement therapy, and methods of treating Pompe's disease using human acid alpha glucosidase.

### CROSS-REFERENCE TO RELATED APPLICATIONS

The present application is a continuation-in-part of U.S. Patent Application Serial No. 60/001,796 filed Aug. 2, 1995, which is now U.S. Pat. No. 6,118,045, granted Sep. 12, 2000 examined as U.S. patent application Ser. No. 08/700,760 filed Jul. 29, 1996 the subject matter of each incorporated by reference herein in their entirety and, the present application is a continuation-in-part of U.S. Patent Application Serial No. 60/111,291 filed Dec. 7, 1998, which is now published as WO/00/34451 on Jun. 15, 2000 from PCT application US99/29042, filed Dec. 6, 1999 the subject matter of each incorporated by reference herein in their entirety.

### TECHNICAL FIELD

The present invention relates to the technical fields of protein chemistry and medicine, and particularly to the production of mannose 6-phosphate containing lysosomal proteins in the milk of transgenic mammals, and administration of the proteins to patients suffering from disease resulting from deficiencies in corresponding endogenous proteins.

### BACKGROUND

Like other secretory proteins, lysosomal proteins are synthesized in the endoplasmic reticulum and transported to the Golgi apparatus. However, unlike most other secretory proteins, the lysosomal proteins are not destined for secretion into extracellular fluids but into an intracellular organelle. Within the Golgi, lysosomal proteins undergo special processing to equip them to reach their intracellular destination. Almost all lysosomal proteins undergo a variety of posttranslational modifications, including glycosylation and phosphorylation via the 6′ position of a terminal mannose group. The phosphorylated mannose residues are recognized by specific receptors on the inner surface of the Trans Golgi Network. The lysosomal proteins bind via these receptors, and are thereby separated from other secretory proteins. Subsequently, small transport vesicles containing the receptor-bound proteins are pinched off from the Trans Golgi Network and are targeted to their intracellular destination. See generally Kornfeld, Biochem. Soc. Trans. 18, 367-374 (1990).

There are over thirty lysosomal diseases, each resulting from a deficiency of a particular lysosomal protein, usually as a result of genetic mutation. See, e.g., Cotran et al., Robbins Pathologic Basis of Disease (4^{th} ed. 1989) (incorporated by reference in its entirety for all purposes). The deficiency in the lysosomal protein usually results in harmful accumulation of a metabolite. For example, in Hurler's, Hunter's, Morquio's, and Sanfilippo's syndromes, there is an accumulation of mucopolysaccharides; in Tay-Sachs, Gaucher, Krabbe, Niemann-Pick, and Fabry syndromes, there is an accumulation of sphingolipids; and in fucosidosis and mannosidosis, there is an accumulation of fucose-containing sphingolipids and glycoprotein fragments, and of mannose-containing oligosaccharides, respectively.

Glycogen storage disease type II (GSD II; Pompe disease; acid maltase deficiency) is caused by deficiency of the lysosomal enzyme acid .alpha.-glucosidase (acid maltase). Acid a-glucosidase (acid maltase) is a enzyme with an essential function in the lysosomal degradation of glycogen to glucose (Rosenfeld, E. L. (1975) Pathol. Biol. 23.71-84). Pathological conditions occur with complete enzyme deficiency or when the functional enzyme is present in low amounts. Massive accumulation of glycogen is observed in the lysosomes, disrupting cellular function (reviewed by Hirschhorn, R. (1995) in The Metabolic and Molecular Basis of Inherited Disease, eds. Scriver, C. R., Beaudet, A. L., Sly, W. S. & Valle, D. (McGraw-Hill New York), 7th Ed., Vol. 2, pp. 2443-2464). Human acid a-glucosidase was discovered in 1963 as the primary defect in Glycogenesis Type II (Pompe's disease) (Hers, H. G. (1963) Biochem. J. 86, 11-16; (Hers, H. G. and De Barsy, Th. (1973) in Lysosomes and Storage Diseases (Hers, H. G., and Van Hoof, F., eds) Pp. 197-216). Glycogenesis Type II is known as an inherited, generalized, glycogen storage disease. Three clinical forms are distinguished: infantile, juvenile and adult. Infantile GSD II has its onset shortly after birth and presents with progressive muscular weakness and cardiac failure. This clinical variant is fatal within the first two years of life. Symptoms in adult and juvenile patients occur later in life, and only skeletal muscles are involved. The patients eventually die due to respiratory insufficiency. Patients may exceptionally survive for more than six decades. There is a good correlation between the severity of the disease and the residual acid .alpha.-glucosidase activity, the activity being 10-20% of normal in late onset and less than 2% in early onset forms of the disease (see Hirschhorn, The Metabolic and Molecular Bases of Inherited Disease (Scriver et al., eds., 7^{th} ed., McGraw-Hill, 1995), pp. 2443-2464).

Since the discovery of lysosomal enzyme deficiencies as the primary cause of lysosomal storage diseases (see, e.g., Hers, Biochem. J. 86, 11-16 (1963)), attempts have been made to treat patients having lysosomal storage diseases by (intravenous) administration of the missing enzyme, i.e., enzyme therapy. For lysosomal diseases other than Gaucher disease the evidence suggests that enzyme therapy is most effective when the enzyme being administered is phosphorylated at the 6′ position of a mannose side chain group. For glycogenesis type II this has been tested by intravenously administering purified acid .alpha.-glucosidase in phosphorylated and unphosphorylated forms to mice and analyzing uptake in muscle tissue. The highest uptake was obtained when mannose 6-phosphate-containing enzyme was used (Van der Ploeg et al., Pediat. Res. 28, 344-347 (1990); J. Clin. Invest. 87, 513-518 (1991)). The greater accumulation of the phosphorylated form of the enzyme can be explained by uptake being mediated by a mannose-6-phosphate receptor present on the surface of muscle and other cells.

Some phosphorylated lysosomal enzymes can, in theory, be isolated from natural sources such as human urine and bovine testis. However, the production of sufficient quantities of enzyme for therapeutic administration is difficult. An alternative way to produce human acid .alpha.-glucosidase is to transfect the acid .alpha.-glucosidase gene into a stable eukaryotic cell line (e.g., CHO) as a cDNA or genomic construct operably linked to a suitable promoter.

Mammalian cellular expression systems are not entirely satisfactory for production of recombinant proteins because of the expense of propagation and maintenance of such cells. An alternative approach to production of recombinant proteins has been proposed by DeBoer et al., WO 91/08216, whereby recombinant proteins are produced in the milk of a transgenic animal. This approach avoids the expense of maintaining mammalian cell cultures and also simplifies purification of recombinant proteins.

Although the feasibility of expressing several recombinant proteins in the milk of transgenic animals has been demonstrated, it was unpredictable whether this technology could be extended to the expression of lysosomal proteins containing mannose 6-phosphate. Because typical secretory proteins like the milk proteins do not contain mannose groups phosphorylated at the 6′ position, it was uncertain whether these cells possessed the necessary complement and activity of enzymes for phosphorylation of substantial amounts of an exogenous lysosomal protein. Further, if such cells did possess the necessary complement of enzymes, it would have appeared likely that phosphorylation would target the phosphorylated lysosomal protein via the mannose 6-phosphate receptor. to an intracellular location rather than to an extracellular location. Substantial intracellular accumulation of a lysosomal protein might have been expected to have harmful or fatal consequences to the mammary gland function of the transgenic animal.

Notwithstanding the above uncertainties and difficulties, the invention provides inter alia healthy transgenic mammals secreting authentically phosphorylated lysosomal proteins in their milk.

Several clinical phenotypes have been observed (reviewed by Hirschhorn, R. (1995) in The Metabolic and molecular bases of inherited disease (Scriver et a/. Eds) Pp. 2443-2464], and some are associated with identified mutations within the human acid a-glucosidase gene (reviewed by Reuser et al, Suppl. 3 (1995) Muscle and Nerve, Pp. S61-S69).

Human acid a-glucosidase is produced in the cell as a 110 kD precursor form. The seven potential N-linked glycosylation sites are probably all used (Hermans et al, (1993) Biochem. J. 289,681-686). The carbohydrate chains are supposed to be of the high mannose type. In the Golgi stack specific mannose residues attached to the precursor are phosphorylated, yielding mannose-6-P. These residues are recognized by the mannose-6-phosphate receptor, which targets proteins to the lysosomes (reviewed by Von Figura & Hasilik, (1986) Ann. Rev. Biochem. 55,167-193; reviewed by Kornfeld, S., (1992) Ann. Rev. Biochem. 61,307-330).

Within the lysosomes, N-and C-terminal processing finally leads, via a 95 kD human acid a-glucosidase intermediate, to the mature 70 and 76 kD enzymes. The mature enzymes are active in the breakdown of glycogen to glucose (Hasilik & Neufeld, J. Biol. Chem. (1980) 255,4937-4946; Hasilik & Neufeld, J. Biol. Chem. (1980) 255, 4946-4950; Martiniuk et al, Arch. Biochem. Biophys. (1984) 231,454-460 ; Reuser et al, J. Biol. Chem. (1985) 260,8336-8341; Reuser et al, J. Clin. Invest. (1987) 79,1689-1699).

In glycogenesis type 11, the lower (or absence of) enzyme activity could be due to many factors, like no or partial mRNA levels, no synthesis of human acid a-glucosidase precursor, or no processing to mature enzyme. Also mature enzyme can be produced, but with lower or no activity (reviewed by Hirschhorn, R. (1995) in The Metabolic and molecular bases of inherited disease (Scriver et al. Eds) Pp. 2443-2464; reviewed by Reuser et al, Muscle & Nerve, Suppl. 3 (1995) Pp S61-S69).

Since the discovery of this and other lysosomal storage diseases, enzyme replacement therapy for Pompe patients has been attempted as a possible treatment. However, the trials were not successful. They were limited in the duration of treatment, and in the amount of enzyme administered. Moreover, either non-human acid a-glucosidase from Aspergillus niger, giving immunological reactions, or “low-uptake” (nonphosphorylated) enzyme from human placenta were used (Baudhuin et al, (1964) Lab. Invest. 13. 1139-1152; Lauer et al, (1968) Pediatrics 42, p. 672; De Barsy et al (1973) In Enzyme Therapy in Genetic Diseases (Eds. Desnick, Bernlohr, Krivit) Williams & Wilkins, Baltimore, Pp. 184-190).

Since the isolation of the gene [Hoefsloot et al (1988) EM80 J. 7, 1697-1704; (Hoefsloot et al (1990) Biochem. J. 272,493-497; Martiniuk et al (1990) DNA Cell Biol. 9,85; Martiniuk et al (1991) DNA Cell Biol. 10,283) expression of recombinant human acid a-glucosidase has been reported.

Recombinant human acid a-glucosidase made in baculovirus-infected insect cells was active but not taken up efficiently by Pompe patient's fibroblasts (Martiniuk et al. (1992) DNA Ce//Biol. 11, 701-706). Fuller et al. (1995) Eur. J. Biochem. 234,903-909) and Van Hove et al ((1996) Proc. Natl. Acad. Sci. USA. 93,65-70), have reported expression in the medium of human precursor acid a-glucosidase of cDNA-transfected Chinese hamster ovary cells.

Acid-a-glucosidase has been purified from a variety of tissues (see review of Hirschhorn, R. (1995) in The Metabolic and molecular bases of inherited disease (Scriver et a/. Eds) Pp. 2443-2464). Many reported procedures are based on two properties of the enzyme: (1) the enzyme is N-glycosylated (predominantly high mannose), so the lectin Concanavalin A coupled to a matrix like Sepharose can be used; and (2) the enzyme has affinity for (1,4 á and (1,6 a-glycosidic linkages, so the enzyme under certain conditions is retarded on a gel-filtration matrices like Sephadex (contains (1,6 linkages) resulting in an affinity type of purification. A number of examples of methods to purify acid a-glucosidase from various tissues are given below.

Jeffrey et al ((1970) Biochem. 9,1403-1415) report the purification of the enzyme from rat liver. After homogenization and centrifugation, the lysosomes were disrupted, and the supernatant, obtained after high-speed centrifugation, was precipitated with 42% ammonium sulphate. The pellet, was resuspended, dialyzed, and loaded on a Sephadex G-100 column. The a-glucosidase fractions from the column were loaded on a weak anion exchange column, and bound enzyme was eluted with 250 mM KCl. The purified enzyme was lyophilized.

Palmer ((1971) Biochem. J. 124,701-711) report the purification of acid a-glucosidase from rabbit muscle. Minced rabbit muscle was washed to remove blood components, homogenized, freeze/thawed, centrifuged, and the precipitate was re-extracted. The combined supernatant were acidified, again centrifuged, and the supernatant was first precipitated with 30% ammonium sulphate. The supernatant was precipitated again, now with 60% ammonium sulphate. The pellet was dissolved in low salt buffer and dialyzed. After freeze/drying, the enzyme was loaded on a Sephadex G-100 column for further purification.

Schram et al ((1979) Biochim. Biophys. Acta 567,370-383) report purification of acid a-glucosidase from human liver. After homogenization and high-speed centrifugation, the supernatant was loaded on a concanavalin A column. Bound enzyme was eluted with 1 M methyl-glucoside, concentrated, dialyzed, and loaded on a S-200 gel-filtration column to obtain purified enzyme.

Martiniuk et al (1984) Arch. Biochem. Biophys. 231, 454) report the purification of acid a-glucosidase from human placenta. After homogenization and centrifugation, the supernatant was loaded on a CM-Sepharose column, essentially to remove hemoglobin. After centrifugation at 27,000 g (15 min), the homogenate was precipitated with 80% ammonium sulphate, centrifuged, and the supernatant was dialyzed, again centrifuged and loaded on a Sephadex G-100 column to obtain purified enzyme.

Reuser et al ((1985) J. Biol. Chem. 260,8336-8341) report the purification of acid a-glucosidase from human placenta. After homogenization and centrifugation, the supernatant was filtered, and loaded on a Concanavalin A Sepharose column. Bound enzyme was eluted with 1 M methyl glucoside, concentrated, dialyzed, and gain concentrated by ultrafiltration before loading on a Sephadex G-200 column. The retarded enzyme was collected from the column and stored frozen.

Lin et al ((1992) Hybridoma 11,493) report the purification of acid a-glucosidase from human urine. The urine was concentrated by ultrafiltration, followed by Concanavalin A column chromatography. Eluted enzyme was precipitated with 80% ammonium sulphate. The pellet was redissolved in PBS, and loaded on a Sephadex G-100 column. The enzyme eluting from the column was again precipitated with 80% ammonium sulphate, and the redissolved pellet was loaded on a DEAE anion column. Bound enzyme was eluted with 0.1 M NaCl buffer. A 70 kD enzyme was visualized on SDS-PAGE.

Fuller et al ((1995) Eur. J. Biochem. 234,903-909) report the purification of recombinant human acid a-glucosidase from the medium of cDNA-transfected Chinese hamster ovary cells. After clarifying the culture medium by low-speed centrifugation, the pH is adjusted to 6.6, and the medium was run over a Concanavalin A sepharose column. Recombinant human acid a-glucosidase is eluted with 1 M methyl-glucoside buffer and concentrated by ultrafiltration. The concentrate is loaded on a Sephadex G-100 column and fractionated at a low flow rate to obtain purified human acid a-glucosidase. Van Hove et al [(1996) Proc. Natl. Acad. Sci. USA. 93,65-70] report the isolation of recombinant human acid (a-glucosidase produced in the medium of transfected CHO cells using similar techniques.

Van Hove et al ((1997) Biochem. Mol. Biol. Int. 43,613-623) report the isolation of recombinant human acid a-glucosidase produced in the medium of transfected CHO cells using the following techniques: after addition of a suitable binding buffer, the medium was loaded on a Concanavalin A column. a-glucosidase was eluted with a 1 M methyl glucoside buffer. Ammonium-sulphate was added, and the sample was loaded on a Phenyl Sepharose HP column. The column was washed, and contaminating proteins were eluted with a gradient of 25-45% elution buffer (20 mM acetate pH 5.3).

Subsequently, a-glucosidase was eluted with a gradient to 100% elution buffer. The enzyme containing fractions were concentrated by ultrafiltration (Amicon stirred bar cell, YM30 membrane), and the enzyme was applied to a Superdex 200 prep grade column. Enzyme was eluted isocratically with 25 mM NaCI, 20 mm acetate buffer pH 4.6 at a low flow rate of 2.5 ml/min.

Enzyme containing solutions were pooled, dia-filtered in the stirred bar cell against a 10 mM NaCI, 25 mM histidine pH 5.5. After loading the sample on a Source Q column, the column was washed with 2% elution buffer (500 mM NaCI, 25 mM histidine pH 5.5) and bound acid a-glucosidase was eluted with a gradient of 24% elution buffer.

All of the above methods are capable of achieving purification of human acid a-glucosidase for therapeutic use. Use of concanavalin A is disadvantageous because it is mitogenic to human lymphocytes and can also give rise to allergy problems (Mody et al. (1995) J. Pharmacol. Toxicol. Methods 33, 1-10). Processing of fractions containing acid a-glucosidase on gel filtration columns, i.e. Sephadex, is also an option but is time consuming and cumbersome for large-scale operation.

### SUMMARY OF THE INVENTION

In one aspect, the invention provides transgenic nonhuman mammals producing a lysosomal protein in their milk. The lysosomal protein is phosphorylated at the 6′ position of a mannose side chain residue. The transgenic mammals have a transgene in their genome. The transgene comprises a mammary-gland specific promoter, a mammary-gland specific enhancer, a secretory DNA segment encoding a signal peptide functional in mammary secretory cells of the transgenic nonhuman mammal, and a recombinant DNA segment encoding a lysosomal protein, usually an enzyme. The recombinant DNA segment is operably linked to the secretory DNA segment to form a secretory-recombinant DNA segment. The secretory-recombinant DNA segment is in turn operably linked to the promoter and to the enhancer. The transgene, in an adult form of the nonhuman mammal or a female descendant of the nonhuman mammal, is capable of expressing the secretory-recombinant DNA segment in the mammary secretory cells to produce a form of the lysosomal protein that is processed and secreted by the mammary secretory cells into milk as a mannose 6-phosphate containing lysosomal protein. The concentration of the mannose 6-phosphate containing lysosomal protein in the milk is usually at least 100 .mu.g/ml. One useful enzyme expressed by such animals is acid .alpha.-glucosidase. Preferred animals for use in the invention include mice, rabbits, goats, sheep, porcines or bovines. The recombinant DNA segment can be cDNA, genomic or a hybrid of the two. In some transgenes, the secretory DNA segment is from the lysosomal protein gene encoded by the recombinant DNA segment.

In another aspect, the invention provides methods for producing a mannose 6-phosphate containing lysosomal protein in the milk of a transgenic mammal. The methods entails recovering milk from the adult form of the transgenic nonhuman mammals described above. Optionally, the mannose 6-phosphate containing protein can be purified from the milk. The purified protein can then be mixed with a pharmaceutical carrier for intravenous, intradermal, intramuscular or oral administration.

In another aspect, the invention provides milk from the transgenic nonhuman mammals described above comprising the mannose 6-phosphate containing lysosomal protein.

In another aspect, the invention provides methods of treating a patient with Pompe's disease. Such methods entail administering to the patient a therapeutically effective amount of human acid alpha glucosidase. The dosage is preferably at least 10 mg/kg body weight per week. In some methods, the dosage is at least 60 mg/kg body weight per week or at least 120 mg/kg body weight per week. In some methods, such dosages are administered on a single occasion per week and in other methods on three occasions per week. In some methods, the treatment is containued for at least 24 weeks.

Administration is preferably intravenous. The human acid alpha glucosidase is preferably obtained in the milk of a nonhuman transgenic mammal, and is preferably predominatly in a 110 kD form.

The methods can be used for treating patients with infantile, juvenile or adult Pompe's disease. In some methods of treating infantile Pompe's disease efficacy is indicated by a patient surviving to be at least one year old.

In some methods, levels of human acid alpha glucosidase are monitored in the recuperated patient. Optionally, a second dosage of human acid alpha glucosidase can be administered if the level of alpha-glucosidase falls below a threshold value in the patient.

In some methods, the human alpha glucosidase is administered intravenously and the rate of administration increases during the period of administration.

In some methods, the rate of administration increases by at least a factor of ten during the period of administration. In some methods, the rate of administration increases by at least a factor of ten within a period of five hours. In some methods, the patient is administered a series of at least four dosages, each dosage at a higher strength than the previous dosage. In some methods, the dosages are a first dosage of 0.03-3 mg/kg/hr, a second dosage of0.3-12 mg/kg/hr, a third dosage of 1-30 mg/kg/hr and a fourth dosage of 2-60 mg/kg/hr. In some methods, the dosages are a first dosage of 0.1-1 mg/kg/hr, a second dosage of 1-4 mg/kg/hr, a third dosage of 3-10 mg/kg/hr and a fourth dosage of 6-20 mg/kg/hr. In some methods, the dosages are a first dosage of 0.25-4 mg/kg/hr, a second dosage of 0.9-1.4 mg/kg/hr, a third dosage of 3.6-5.7 mg/kg/hr and a fourth dosage of 7.211.3 mg/kg/hr. In some methods, the dosages are a first dosage of 0.3 mg/kg/hr, a second dosage of 1 mg/kg/hr, a third dosage of 4 mg/kg/hr and a fourth dosage of 12 mg/kg/hr.

In some methods, the first, second, third and fourth dosages are each administered for periods of 15 min to 8 hours.

In some methods, the first, second, third and fourth dosages are administered for periods of 1 hr, 1 hr, 0.5 hr and 3 hr respectively.

In another aspect, the invention provides a pharmaceutical composition comprising human acid alpha glucosidase, human serum albumin, and a sugar in a physiologically acceptable buffer in sterile form. Some such compositions comprise human acid alpha glucosidase. human serum albumin. and glucose in sodium phosphate buffer. Some compositions comprise alpha glucosidase, mannitol and sucrose in an aqueous solution. In some compositions, the sugar comprises mannitol and sucrose and the concentration of mannitol is 1-3% w/w of the aqueous solution and the concentration of sucrose is 0.1 to 1% w/w of the aqueous solution. In some compositions, the concentration of mannitol is 2% w/w and the concentration of sucrose is 0.5% w/w.

The invention further provides a lyophilized composition produced by lyophilizing a pharmaceutical composition comprising human acid glucosidase, mannitol and sucrose in aqueous solution. Such a composition can be prepared by lyophilizing a first composition comprising human acid alpha-glucosidase, mannitol, sucrose and an aqueous solution to produce a second composition; and reconstituting the lyophilized composition in saline to produce a third composition. In some such compositions, the human acid alpha-glucosidase is at 5 mg/ml in both the first and third composition, the mannitol is at 2 mg/ml in the first composition, the sucrose is at 0.5 mg/ml in the first composition, and the saline used in the reconstituting step is 0.9% w/w.

The invention provides for the use of human acid a-glucosidase as hereinbefore defined for the manufacture of a medicament for treatment of human acid a-glucosidase deficiency. In twelfth aspect the invention provides for the use of human acid a-glucosidase as hereinbefore defined for the manufacture of a medicament for intravenous administration for the treatment of human acid a-glucosidase deficiency.

### BRIEF DESCRIPTION OF THE DRAWINGS

FIG. 1: A transgene containing acid .alpha.-glucosidase cDNA. The .alpha.s1-casein exons are represented by open boxes; .alpha.-glucosidase cDNA is represented by a shaded box. The .alpha.s1-casein intron and flanking sequences are represented by a thick line. A thin line represents the IgG acceptor site. The transcription initiation site is marked ( ), the translation initiation site (ATG), the stopcodon (TAG) and the polyadenylation site (pA).

FIG. 2 (panels A, B, C): Three transgenes containing acid .alpha.-glucosidase genomic DNA. Dark shaded areas are .alpha.s1 casein sequences, open boxes represent acids .alpha-glucosidase exons, and the thin line between the open boxes represents .alpha.-glucosidase introns. Other symbols are the same as in FIG. 1.

FIG. 3 (panels A, B, C): Construction of genomic transgenes. The .alpha.-glucosidase exons are represented by open boxes; the .alpha.-glucosidase introns and nontranslated sequences are indicated by thin lines. The pKUN vector sequences are represented by thick lines.

FIG. 4 (panels A and B). Detection of acid .alpha.-glucosidase in milk of transgenic mice by Western blotting.

FIG. 5. Chromatography profile of rabbit whey on a Q Sepharose FF column.

A whey fraction from rabbit (line 60) milk (about 550 ml), prepared by tangential flow filtration (TFF) of the (diluted) skimmed milk, was incubated with solvent/detergent (1% Tween-80,0.3% TnBP), and loaded on a Q Sepharose FF column (Pharmacia XK-50 column, 18 cm bed height; 250 cm/hr flow rate). The column was washed with (7) column volumes (cv) of buffer A (20 mM sodium phosphate buffer pH 7.0), and the human acid a-glucosidase fraction was eluted with 3.5 cv buffer A, containing 100 mM sodium chloride. All strongly bound proteins were eluted with about 3 cv 100% buffer B (1 M NaCl, 20 mM sodium phosphate buffer pH 7.0). All column chromatography was controlled by the AKTA system of Pharmacia. Protein was detected on-line by measuring the absorbance at 280 nm.

FIG. 6. Chromatography profile of Q Sepharose FF-purified recombinant human a-glucosidase fraction on a Phenyl HP Sepharose column.

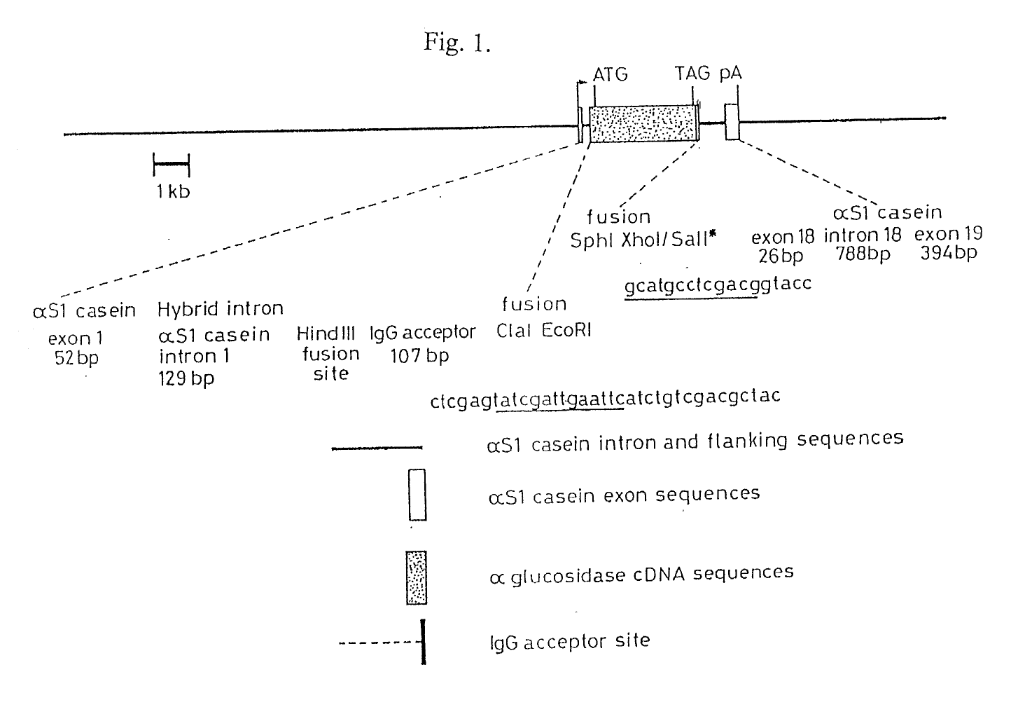

One volume of 1 M ammonium sulphate was added to the Q Sepharose FF human acid a-glucosidase eluate (obtained with 100 mM sodium chloride, 20 mM sodium phosphate buffer pH 7.0 step; fraction F3 of FIG. 1) while stirring continuously. This sample was loaded on a Phenyl HP Sepharose column (Pharmacia XK-50 column 14 cm bed height; 150 cm/hr flow rate) at room temperature (loaded 1-1.2 mg a-glucosidase/ml Sepharose). Before loading, the column was equilibrated in 0.5 M ammonium sulphate, 50 mM sodium phosphate buffer pH 6.0 (=buffer C). After loading the sample, the column was washed with 2 cv of buffer C to remove contaminating proteins like transferrin and serum albumin. Most recombinant human acid a-glucosidase was eluted from the Phenyl HP column with 4 cv buffer D (=50 mM sodium phosphate at pH 6.0 buffer). The strongest bound proteins were eluted first with water, then with 20% ethanol.

FIG. 7. Chromatography profile of a (Phenyl HP Sepharose-purified) recombinant human a-glucosidase fraction on Source Phenyl 15 column.

A 2 M ammonium-sulphate, 50 mM sodium phosphate buffer, pH 7.0 was added to the human acid a-glucosidase eluate from the Phenyl HP column (fraction F4 from FIG. 6), until a final concentration of 0.85 M ammonium sulphate was reached. The solution was stirred continuously and mildly. The eluate was loaded on a Source Phenyl 15 column (Pharmacia Fineline 100 column, 15 cm bed height ; 76 cm/hr flow rate) pre-equilibrated in 0.85 M ammonium sulphate, 50 mM sodium phosphate pH 7.0 buffer (=buffer E). About 2 mg of acid a-glucosidase can be loaded per ml Source 15 Phenyl in this column. After loading the sample, recombinant human acid a glucosidase was eluted from the (Source 15 Phenyl) column with 10 cv of a linear gradient from 100% buffer E to 100% buffer F (buffer F=50 mM sodium phosphate buffer, pH 7.0). Careful pooling of the elution fraction is required (based on purity profiles of the column fractions on SDS-PAGE using Coomassie Brillant Blue staining) to obtain highly purified recombinant acid a-glucosidase. Residual bound proteins were eluted from the column, first with water, and then with 20% ethanol.

FIG. 8. SDS-PAGE analysis of various fractions during the acid a-glucosidase purification procedure.

Various fractions obtained during a recombinant human acid a-glucosidase purification from rabbit milk (line 60) were diluted in non-reduced SDS sample buffer. The samples were boiled for 5 minutes and loaded on a SDS-PAGE gradient gel (4-12%, Novex). Proteins were stained with Coomassie Brillant Blue. Lane 1: Full rabbit milk (40 ug); 2. Whey after TFF of skimmed milk (40, ug); 3. Acid a-glucosidase eluate fraction from the Q Sepharose FF column (30 ug); 4. Acid aglucosidase eluate fraction from the Phenyl HP column (5 ug); 5. Acid aglucosidase eluate fraction from the Source 15 Phenyl column (5 ug). The letters refer to protein bands which were identified as: a.) rabbit immunoglobulins; b.) unknown protein; c.) recombinant human acid aglucosidase precursor (doublet under these SDS-PAGE conditions); d.) rabbit transferrin; e.) rabbit serum albumin; f.) rabbit caseins; g.) rabbit Whey Acidic Protein (WAP), possibly a dimer; h.) rabbit Whey Acidic Protein (WAP), monomer; i.) unknown protein, possibly a rabbit WAP variant, or a-lactalbumin; j.) dimer or recombinant human acid a-glucosidase precursor (doublet under these SDS-PAGE conditions); k.) unknown protein (rabbit transferrin, or processed recombinant human acid a-glucosidase).

FIG. 9. HPLC size exclusion profile of purified recombinant human acid aglucosidase precursor. Recombinant human acid a-glucosidase precursor was purified from transgenic rabbit milk by defatting milk, TFF of skimmed milk, Q FF chromatography, Phenyl HP chromatography. Source 15 Phenyl chromatography, and final filtration. The sample was prepared for the HP SEC chromatography run as described in Example 5.

FIG. 10. Binding of 1251 human acid a-glucosidase precursor to various metal-chelating and lectin Sepharoses.

Purified human acid a-glucosidase precursor from rabbit line 60 was radio-labeled with 1251 as described in Example 5. Binding of the labeled enzyme to the metal-chelating Sepharoses (Fe2+, Fe3+, Cu2+, Zn2+, glycine, and control) and to the lectin Sepharoses (Concanavalin A and lentil) was done as described in Example 1. Two washing procedures were tested: either a wash with PBS, 0.002% Tween-20 buffer, or a wash with PBS, 0.1% Tween-20,0.5 M sodium chloride buffer. The binding percentages relate to the total amount of radiolabel added to the tubes.

FIG. 11. Chromatographic elution profiles of acid a-glucosidase-containing fractions on various HIC columns.

Purified acid a-glucosidase 110 kDa precursor or mature 76 kDa acid a-glucosidase (A and B; both 5 ug; recombinant from transgenic mouse milk line 2585) were analyzed on a 1 ml Butyl 4 Fast Flow Sepharose or Octyl 4 Fast Flow Sepharose HiTrap column (Pharmacia, Sweden). A transgenic (line 60;-0-) and non-transgenic (--)′ whey fraction (prepared by 20,000 g, 60 min centrifugation) were also analyzed on a butyl column (both 200 ul, 25 fold diluted; C). Also a Q Fast Flow fraction (eluted at 100 ut salt from the column; see FIG. 1) of transgenic (line 60 ;-0-) and non-transgenic (-) whey were loaded on an ether column (both 200 ul, 25-fold diluted; Toyopearl Ether 650 M (TosoHaas) in a 2.5 ml, 5 cm bed height column; C). The results indicate a strong binding of acid a-glucosidase to the HIC columns (A and B). Most whey proteins do not bind (C). A nearly pure acid a-glucosidase was obtained after loading a Q Fast Flow eluate on an ether column (D), where most of the contaminating proteins like serum albumin and transferrin do not bind (SDS-PAGE gels not shown). The binding buffer in A, B, and C was M ammonium sulphate, 50 mM sodium phosphate pH 7.0. The binding buffer in D was 1.5 M ammonium sulphate, 50 mM sodium phosphate pH 7.0. The flow rate was 1 ml/min. Bound protein was eluted with a linear salt gradient to 50 mM sodium phosphate pH 7.0 in 30 min. All column chromatography was controlled by the AKTA system of Pharmacia. Protein was detected on-line by measuring the absorbance at 280 nm (0.2 cm flow cell). The conductivity was measured on line. mAU=milli-Absorbance units, mS/cm=milli-Siemens/cm.

FIG. 12 Chromatography profiles of transgenic and non-transgenic whey fractions on a Hydroxylapatite column. Transgenic (----) and non-transgenic (-.-.-.-) rabbit whey, obtained after skimming (by centrifugation) and casein removal (by TFF), were loaded on a Amberchrome column (4.6×150 mm) containing Macro-Prep ceramic hydroxylapatite type I (40 Ltm beads; BioRad) connected to a FPLC system of Pharmacia. Whey fractions obtained after TFF were diluted 5-fold in buffer A (10 mM NaPi pH 6.8), and 0.2 ml was loaded on the column pre-equilibrated in buffer A. The flow rate was 2 ml/min. After loading, bound protein was eluted with a gradient to 500 mM NaPj pH 6.8 in 10 column volumes. Protein was detected by measuring the absorbance at 280 nm (flow cell is 2 mm).

FIG. 13. SDS-PAGE analysis of whey fractions from the hydroxylapatite column. Transgenic and non-transgenic rabbit whey were loaded on the Macro-Prep ceramic hydroxylapatite type) column as described in FIG. 12. Flow through and eluate fractions were obtained, which were analyzed on SDS-PAGE (for details of the gels see FIG. 8). A. silver stained SDS PAGE of transgenic whey run on hydroxylapatite; B. silver stained SDS PAGE of non-transgenic whey. Up to 6 g protein was loaded.

FIGS. 14 to 19 are chromatograms of hydroxylapatite chromatography separations of transgenic whey samples in which the samples were loaded on to the column at sodium phosphate buffer (NaPi) concentrations of 5, 10, 20, 30, 40 or 50 mM respectively. The pH of the buffer was 7.0. The chromatograms show the gradient of sodium phosphate eluting buffer to 400 mM, the AZSO and the pH of the eluate and the fractions collected.

FIGS. 20 to 23 are chromatograms of hydroxylapatite chromatography separations as in FIGS. 14 to 19 above except that the pH of the sample was varied whilst the NaPi buffer concentration was retained at 5 mM. The pH of the samples fractionated were pH 6.0, 7.0 and 7.5 respectively.

FIG. 24 is a chromatogram of an industrial (pilot) scale separation of transgenic milk whey on Q Sepharose FF.

FIG. 25 is a chromatogram of hydroxylapatite column chromatography of 0.1 M eluate from the Q Sepharose FF column.

FIG. 26 is a silver stained SDS-PAGE gel of flow through fractions from a series of hydroxylapatite chromatography separations of 0.1M eluates of Q Sepharose FF.

### DEFINITIONS

The term “substantial identity” or “substantial homology” means that two peptide sequences, when optimally aligned, such as by the programs GAP or BESTFIT using default gap weights, share at least 65 percent sequence identity, preferably at least 80 or 90 percent sequence identity, more preferably at least 95 percent sequence identity or more (e.g., 99 percent sequence identity). Preferably, residue positions which are not identical differ by conservative amino acid substitutions.

The term “substantially pure” or “isolated” means an object species, e.g. human acid a-glucosidase, has been identified and separated and/or recovered from a component of its natural environment. Usually, the object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 to 90 percent by weight of all macromolecular species present in the composition. Most preferably, the object species is purified to 95%, 99%, or 99.9% purity or essential homogeneity (contaminant species cannot be detected in the composition by conventional detection methods) wherein the composition consists essentially of derivatives of a single macromolecular species.

A DNA segment is operably linked when placed into a functional relationship with another DNA segment. For example, DNA for a signal sequence is operably linked to DNA encoding a polypeptide if it is expressed as a preprotein that participates in the secretion of the polypeptide; a promoter or enhancer is operably linked to a coding sequence if it stimulates the transcription of the sequence. Generally, DNA sequences that are operably linked are contiguous, and in the case of a signal sequence both contiguous and in reading phase. However, enhancers need not be contiguous with the coding sequences whose transcription they control. Linking is accomplished by ligation at convenient restriction sites or at adapters or linkers inserted in lieu thereof.

An exogenous DNA segment is one foreign to the cell, or homologous to a DNA segment of the cell but in an unnatural position in the host cell genome. Exogenous DNA segments are expressed to yield exogenous polypeptides.

In a transgenic mammal, all, or substantially all, of the germline and somatic cells contain a transgene introduced into the mammal or an ancestor of the mammal at an early embryonic stage.

A low salt buffer means a buffer with a salt concentration less than 100 mM and preferably less than 50 mM. A high salt buffer means a buffer with a salt concentration greater than 300 mM and preferably at least 500 mM.

### DETAILED DESCRIPTION

The invention provides transgenic nonhuman mammals secreting a mannose 6-phosphate containing lysosomal protein into their milk. Secretion is achieved by incorporation of a transgene encoding a lysosomal protein and regulatory sequences capable of targeting expression of the gene to the mammary gland. The transgene is expressed, and the expression product posttranslationally modified within the mammary gland, and then secreted in milk. The posttranslational modification includes steps of glycosylation and phosphorylation.

A. Lysosomal Genes

The invention provides transgenic nonhuman mammals expressing DNA segments containing any of the more than 30 known genes encoding lysosomal enzymes and other types of lysosomal proteins, including .alpha.-glucosidase, .alpha.-L-iduronidase, iduronate-sulfate sulfatase, hexosaminidase A and B, ganglioside activator protein, arylsulfatase A and B, iduronate sulfatase, heparan N-sulfatase, galactoceramidase, .alpha.-galactosylceramidase A, sphingomyelinase, .alpha.-fucosidase, .alpha.-mannosidase, aspartylglycosamine amide hydrolase, acid lipase, N-acetyl-.alpha.-D-glucosamine-6-sulphate sulfatase, .alpha.- and .beta.-galactosidase, .beta.-glucuronidase, .beta.-mannosidase, ceramidase, galactocerebrosidase, .alpha.-N-acetylgalactosaminidase, and protective protein and others. Transgenic mammals expressing allelic, cognate and induced variants of any of the known lysosomal protein gene sequences are also included. Such variants usually show substantial sequence identity at the amino acid level with known lysosomal protein genes. Such variants usually hybridize to a known gene under stringent conditions or crossreact with antibodies to a polypeptide encoded by one of the known genes.

DNA clones containing the genomic or cDNA sequences of many of the known genes encoding lysosomal proteins are available. (Scott et al., Am. J Hum. Genet. 47, 802-807 (1990); Wilson et al., PNAS 87, 8531-8535 (1990); Stein et al., J. Biol. Chem. 264, 1252-1259 (1989); Ginns et al., Biochem. Biophys. Res. Comm. 123, 574-580 (1984); Hoefsloot et al., EMBO J. 7, 1697-1704 (1988); Hoefsloot et al., Biochem. J. 272, 473-479 (1990); Meyerowitz & Proia, PNAS 81, 5394-5398 (1984); Scriver et al., supra, part 12, pages 2427-2882 and references cited therein)) Other examples of genomic and cDNA sequences are available from GenBank. To the extent that additional cloned sequences of lysosomal genes are required, they may be obtained from genomic or cDNA libraries (preferably human) using known lysosomal protein DNA sequences or antibodies to known lysosomal proteins as probes.

B. Conformation of Lysosomal Proteins

Recombinant lysosomal proteins are preferably processed to have the same or similar structure as naturally occurring lysosomal proteins. Lysosomal proteins are glycoproteins that are synthesized on ribosomes bound to the endoplasmic reticulum (RER). They enter this organelle co-translationally guided by an N-terminal signal peptide (Ng et al., Current Opinion in Cell Biology 6, 510-516 (1994)). The N-linked glycosylation process starts in the RER with the en bloc transfer of the high-mannose oligosaccharide precursor Glc.sub.3 MangGlcNAc.sub.2 from a dolichol carrier. Carbohydrate chain modification starts in the RER and continue in the Golgi apparatus with the removal of the three outermost glucose residues by glycosidases I and II. Phosphorylation is a two-step procedure in which first N-acetylglucosamine-1-phosphate is coupled to select mannose groups by a lysosomal protein specific transferase, and second, the N-acetylglucosamine is cleaved by a diesterase (Goldberg et al., Lysosomes: Their Role in Protein Breakdown (Academic Press Inc., London, 1987), pp. 163-191). Cleavage exposes mannose 6-phosphate as a recognition marker and ligand for the mannose 6-phosphate receptor mediating transport of most lysosomal proteins to the lysosomes (Kornfeld, Biochem. Soc. Trans. 18, 367-374 (1992)).

In addition to carbohydrate chain modification, most lysosomal proteins undergo proteolytic processing, in which the first event is removal of the signal peptide. The signal peptide of most lysosomal proteins is cleaved after translocation by signal peptidase after which the proteins become soluble. There is suggestive evidence that the signal peptide of acid .alpha.-glucosidase is cleaved after the enzyme has left the RER, but before it has entered the lysosome or the secretory pathway (Wisselaar et al., J. Biol. Chem. 268, 2223-2231 (1993)). The proteolytic processing of acid .alpha.-glucosidase is complex and involves a series of steps in addition to cleavage of the signal peptide taking place at various subcellular locations. Polypeptides are cleaved off at both the N and C terminal ends, whereby the specific catalytic activity is increased. The main species recognized are a 110/100 kDa precursor, a 95 kDa intermediate and 76 kDa and 70 kDa mature forms. (Hasilik et al., J. Biol. Chem. 255, 4937-4945 (1980); Oude Elferink et al., Eur. J. Biochem. 139, 489-495 (1984); Reuser et al., J. Biol. Chem. 260, 8336-8341 (1985); Hoefsloot et al., EMBO J. 7, 1697-1704 (1988)). The post translational processing of natural human acid .alpha.-glucosidase and of recombinant forms of human acid .alpha.-glucosidase as expressed in cultured mammalian cells like COS cells, BHK cells and CHO cells is similar (Hoefsloot et al., (1990) supra; Wisselaar et al., (1993) supra.

Authentic processing to generate lysosomal proteins phosphorylated at the 6′ position of the mannose group can be tested by measuring uptake of a substrate by cells bearing a receptor for mannose 6-phosphate. Correctly modified substrates are taken up faster than unmodified substrates, and in a manner whereby uptake of the modified substrate can be competitively inhibited by addition of mannose 6-phosphate.

C. Transgene Design

Transgenes are designed to target expression of a recombinant lysosomal protein to the mammary gland of a transgenic nonhuman mammal harboring the transgene. The basic approach entails operably linking an exogenous DNA segment encoding the protein with a signal sequence, a promoter and an enhancer. The DNA segment can be genomic, minigene (genomic with one or more introns omitted), cDNA, a YAC fragment, a chimera of two different lysosomal protein genes, or a hybrid of any of these. Inclusion of genomic sequences generally leads to higher levels of expression. Very high levels of expression might overload the capacity of the mammary gland to perform posttranslation modifications, and secretion of lysosomal proteins. However, the data presented below indicate that substantial posttranslational modification occurs including the formation of mannose 6-phosphate groups, notwithstanding a high expression level in the mg/ml range. Substantial modification means that at least about 10, 25, 50, 75 or 90% of secreted molecules bear at least one mannose 6-phosphate group. Thus, genomic constructs or hybrid cDNA-genomic constructs are generally preferred. In genomic constructs, it is not necessary to retain all intronic sequences. For example, some intronic sequences can be removed to obtain a smaller transgene facilitating DNA manipulations and subsequent microinjection. See Archibald et al., WO 90/05188 (incorporated by reference in its entirety for all purposes). Removal of some introns is also useful in some instances to reduce expression levels and thereby ensure that posttranslational modification is substantially complete. It is also possible to delete some or all of noncoding exons. In some transgenes, selected nucleotides in lysosomal protein encoding sequences are mutated to remove proteolytic cleavage sites.

Because the intended use of lysosomal proteins produced by transgenic mammals is usually administration to humans, the species from which the DNA segment encoding a lysosomal protein sequence is obtained is preferably human. Analogously if the intended use were in veterinary therapy (e.g., on a horse, dog or cat), it is preferable that the DNA segment be from the same species.

The promoter and enhancer are from a gene that is exclusively or at least preferentially expressed in the mammary gland (i.e., a mammary-gland specific gene). Preferred genes as a source of promoter and enhancer include .beta.-casein, .kappa.-casein, .alpha.S1-casein, .alpha.S2-casein, .beta.-lactoglobulin, whey acid protein, and .alpha.-lactalbumin. The promoter and enhancer are usually but not always obtained from the same mammary-gland specific gene. This gene is sometimes but not necessarily from the same species of mammal as the mammal into which the transgene is to be expressed. Expression regulation sequences from other species such as those from human genes can also be used. The signal sequence must be capable of directing the secretion of the lysosomal protein from the mammary gland. Suitable signal sequences can be derived from mammalian genes encoding a secreted protein. Surprisingly, the natural signal sequences of lysosomal proteins are suitable, notwithstanding that these proteins are normally not secreted but targeted to an intracellular organelle. In addition to such signal sequences, preferred sources of signal sequences are the signal sequence from the same gene as the promoter and enhancer are obtained. Optionally, additional regulatory sequences are included in the transgene to optimize expression levels. Such sequences include 5′ flanking regions, 5′ transcribed but untranslated regions, intronic sequences, 3′ transcribed but untranslated regions, polyadenylation sites, and 3′ flanking regions. Such sequences are usually obtained either from the mammary-gland specific gene from which the promoter and enhancer are obtained or from the lysosomal protein gene being expressed. Inclusion of such sequences produces a genetic milieu simulating that of an authentic mammary gland specific gene and/or that of an authentic lysosomal protein gene. This genetic milieu results in some cases (e.g., bovine .alpha.S1-casein) in higher expression of the transcribed gene Alternatively, 3′ flanking regions and untranslated regions are obtained from other heterologous genes such as the .beta.-globin gene or viral genes. The inclusion of 3′ and 5′ untranslated regions from a lysosomal protein gene, or other heterologous gene can also increase the stability of the transcript.

In some embodiments, about 0.5, 1,5, 10, 15, 20 or 30 kb of 5′ flanking sequence is included from a mammary specific gene in combination with about 1, 5, 10, 15, 20 or 30 kb or 3′ flanking sequence from the lysosomal protein gene being expressed. If the protein is expressed from a cDNA sequence, it is advantageous to include an intronic sequence between the promoter and the coding sequence. The intronic sequence is preferably a hybrid sequence formed from a 5′ portion from an intervening sequence from the first intron of the mammary gland specific region from which the promoter is obtained and a 3′ portion from an intervening sequence of an IgC intervening sequence or lysosomal protein gene. See DeBoer et al., WO 91/08216 (incorporated by reference in its entirety for all purposes).

A preferred transgene for expressing a lysosomal protein comprises a cDNA-genomic hybrid lysosomal protein gene linked 5′ to a casein promoter and enhancer. The hybrid gene includes the signal sequence, coding region, and a 3′ flanking region from the lysosomal protein gene. Optionally, the cDNA segment includes an intronic sequence between the 5′ casein and untranslated region of the gene encoding the lysosomal protein. Of course, corresponding cDNA and genomic segments can also be fused at other locations within the gene provided a contiguous protein can be expressed from the resulting fusion.

Other preferred transgenes have a genomic lysosomal protein segment linked 5′ to casein regulatory sequences. The genomic segment is usually contiguous from the 5′ untranslated region to the 3′ flanking region of the gene. Thus, the genomic segment includes a portion of the lysosomal protein 5′ untranslated sequence, the signal sequence. alternating introns and coding exons, a 3′ untranslated region, and a 3′ flanking region. The genomic segment is linked via the 5′ untranslated region to a casein fragment comprising a promoter and enhancer and usually a 5′ untranslated region.

DNA sequence information is available for all of the mammary gland specific genes listed above, in at least one, and often several organisms. See, e.g., Richards et al., J. Biol. Chem. 256, 526-532 (1981) (.alpha.-lactalbumin rat); Campbell et al., Nucleic Acids Res. 12, 8685-8697 (1984) (rat WAP); Jones et al., J. Biol. Chem. 260, 7042-7050 (1985)) (rat .beta.-casein); Yu-Lee & Rosen, J. Biol. Chem. 258, 10794-10804 (1983) (rat .gamma.-casein)); Hall, Biochem. J. 242, 735-742 (1987) (.alpha.-lactalbumin human); Stewart, Nucleic Acids Res. 12, 389 (1984) (bovine .alpha.s1 and K casein cDNAs); Gorodetsky et al., Gene 66, 87-96 (1988) (bovine .beta. casein); Alexander et al., Eur. J. Biochem. 178, 395-401 (1988) (bovine .kappa. casein); Brignon et al., FEBS Lett. 188, 48-55 (1977) (bovine .alpha.S2 casein); Jamieson et al., Gene 61, 85-90 (1987), Ivanov et al., Biol. Chem. Hoppe-Seyler 369, 425-429 (1988), Alexander et al., Nucleic Acids Res. 17, 6739 (1989) (bovine .beta. lactoglobulin); Vilotte et al., Biochimie 69, 609-620 (1987) (bovine .alpha.-lactalbumin) (incorporated by reference in their entirety for all purposes). The structure and function of the various milk protein genes are reviewed by Mercier & Vilotte, J. Dairy Sci. 76, 3079-3098 (1993) (incorporated by reference in its entirety for all purposes). To the extent that additional sequence data might be required, sequences flanking the regions already obtained could be readily cloned using the existing sequences as probes. Mammary-gland specific regulatory sequences from different organisms are likewise obtained by screening libraries from such organisms using known cognate nucleotide sequences, or antibodies to cognate proteins as probes. General strategies and exemplary transgenes employing .alpha.S1-casein regulatory sequences for targeting the expression of a recombinant protein to the mammary gland are described in more detail in DeBoer et al., WO 91/08216 and WO 93/25567 (incorporated by reference in their entirety for all purposes). Examples of transgenes employing regulatory sequences from other mammary gland specific genes have also been described. See, e.g., Simon et al., Bio/Technology 6, 179-183 (1988) and WO88/00239 (1988) (.beta.-lactoglobulin regulatory sequence for expression in sheep); Rosen, EP 279,582 and Lee et al., Nucleic Acids Res. 16, 1027-1041 (1988) (.beta.-casein regulatory sequence for expression in mice); Gordon, Biotechnology 5, 1183 (1987) (WAP regulatory sequence for expression in mice); WO 88/01648 (1988) and Eur. J. Biochem. 186, 43-48 (1989) (.alpha.-lactalbumin regulatory sequence for expression in mice) (incorporated by reference in their entirety for all purposes).

The expression of lysosomal proteins in the milk from transgenes can be influenced by co-expression or functional inactivation (i.e., knock-out) of genes involved in post translational modification and targeting of the lysosomal proteins. The data in the Examples indicate that surprisingly mammary glands already express modifying enzymes at sufficient quantities to obtain assembly and secretion of mannose 6-phosphate containing proteins at high levels. However, in some transgenic mammals expressing these proteins at high levels, it is sometimes preferable to supplement endogenous levels of processing enzymes with additional enzyme resulting from transgene expression. Such transgenes are constructed employing similar principles to those discussed above with the processing enzyme coding sequence replacing the lysosomal protein coding sequence in the transgene. It is not generally necessary that posttranslational processing enzymes be secreted. Thus, the secretion signal sequence linked to the lysosomal protein coding sequence is replaced with a signal sequence that targets the processing enzyme to the endoplasmic reticulum without secretion. For example, the signal sequences naturally associated with these enzymes are suitable.

D. Transgenesis

The transgenes described above are introduced into nonhuman mammals. Most nonhuman mammals, including rodents such as mice and rats, rabbits, ovines such as sheep and goats, porcines such as pigs, and bovines such as cattle and buffalo, are suitable. Bovines offer an advantage of large yields of milk, whereas mice offer advantages of ease of transgenesis and breeding. Rabbits offer a compromise of these advantages. A rabbit can yield 100 ml milk per day with a protein content of about 14% (see Buhler et al., Bio/Technology 8, 140 (1990)) (incorporated by reference in its entirety for all purposes), and yet can be manipulated and bred using the same principles and with similar facility as mice. Nonviviparous mammals such as a spiny anteater or duckbill platypus are typically not employed.

In some methods of transgenesis, transgenes are introduced into the pronuclei of fertilized oocytes. For some animals, such as mice and rabbits, fertilization is performed in vivo and fertilized ova are surgically removed. In other animals, particularly bovines, it is preferable to remove ova from live or slaughterhouse animals and fertilize the ova in vitro. See DeBoer et al., WO 91/08216. In vitro fertilization permits a transgene to be introduced into substantially synchronous cells at an optimal phase of the cell cycle for integration (not later than S-phase). Transgenes are usually introduced by microinjection. See U.S. Pat. No. 4,873,292. Fertilized oocytes are then cultured in vitro until a pre-implantation embryo is obtained containing about 16-150 cells. The 16-32 cell stage of an embryo is described as a morula. Pre-implantation embryos containing more than 32 cells are termed blastocysts. These embryos show the development of a blastocoel cavity, typically at the 64 cell stage. Methods for culturing fertilized oocytes to the pre-implantation stage are described by Gordon et al., Methods Enzymol. 101, 414 (1984); Hogan et al., Manipulation of the Mouse Embryo: A Laboratory Manual, C.S.H.L. N.Y. (1986) (mouse embryo); and Hammer et al., Nature 315, 680 (1985) (rabbit and porcine embryos); Gandolfi et al. J. Reprod. Fert. 81,23-28 (1987); Rexroad et al., J. Anim. Sci. 66, 947-953 (1988) (ovine embryos) and Eyestone et al. J. Reprod. Fert. 85, 715-720 (1989); Camous et al., J. Reprod. Fert. 72, 779-785 (1984); and Heyman et al. Theriogenology 27, 5968 (1987) (bovine embryos) (incorporated by reference in their entirety for all purposes). Sometimes pre-implantation embryos are stored frozen for a period pending implantation. Pre-implantation embryos are transferred to the oviduct of a pseudopregnant female resulting in the birth of a transgenic or chimeric animal depending upon the stage of development when the transgene is integrated. Chimeric mammals can be bred to form true germline transgenic animals.

Alternatively, transgenes can be introduced into embryonic stem cells (ES). These cells are obtained from preimplantation embryos cultured in vitro. Bradley et al., Nature 309, 255-258 (1984) (incorporated by reference in its entirety for all purposes). Transgenes can be introduced into such cells by electroporation or microinjection. Transformed ES cells are combined with blastocysts from a nonhuman animal. The ES cells colonize the embryo and in some embryos form the germline of the resulting chimeric animal. See Jaenisch, Science, 240, 1468-1474 (1988) (incorporated by reference in its entirety for all purposes). Alternatively, ES cells can be used as a source of nuclei for transplantation into an enucleated fertilized oocyte giving rise to a transgenic mammal.

For production of transgenic animals containing two or more transgenes, the transgenes can be introduced simultaneously using the same procedure as for a single transgene. Alternatively, the transgenes can be initially introduced into separate animals and then combined into the same genome by breeding the animals. Alternatively, a first transgenic animal is produced containing one of the transgenes. A second transgene is then introduced into fertilized ova or embryonic stem cells from that animal. In some embodiments, transgenes whose length would otherwise exceed about 50 kb, are constructed as overlapping fragments. Such overlapping fragments are introduced into a fertilized oocyte or embryonic stem cell simultaneously and undergo homologous recombination in vivo. See Kay et al., WO 92/03917 (incorporated by reference in its entirety for all purposes).

E. Characteristics of Transgenic Mammals

Transgenic mammals of the invention incorporate at least one transgene in their genome as described above. The transgene targets expression of a DNA segment encoding a lysosomal protein at least predominantly to the mammary gland. Surprisingly, the mammary glands are capable of expressing proteins required for authentic posttranslation processing including steps of oligosaccharide addition and phosphorylation. Processing by enzymes in the mammary gland results in phosphorylation of the 6′ position of mannose groups. Lysosomal proteins can be secreted at high levels of at least 10, 50, 100, 500, 1000, 2000, 5000 or 10,000 .mu.g/ml. Surprisingly, the transgenic mammals of the invention exhibit substantially normal health. Secondary expression of lysosomal proteins in tissues other than the mammary gland does not occur to an extent sufficient to cause deleterious effects. Moreover, exogenous lysosomal protein produced in the mammary gland is secreted with sufficient efficiency that no significant problem is presented by deposits clogging the secretory apparatus.

The age at which transgenic mammals can begin producing milk, of course, varies with the nature of the animal. For transgenic bovines, the age is about two-and-a-half years naturally or six months with hormonal stimulation, whereas for transgenic mice the age is about 5-6 weeks. Of course, only the female members of a species are useful for producing milk. However, transgenic males are also of value for breeding female descendants. The sperm from transgenic males can be stored frozen for subsequent in vitro fertilization and generation of female offspring.

F. Recovery of Proteins from Milk

Transgenic adult female mammals produce milk containing high concentrations of exogenous lysosomal protein. The protein can be purified from milk, if desired, by virtue of its distinguishing physical and chemical properties, and standard purification procedures such as precipitation, ion exchange, molecular exclusion or affinity chromatography. See generally Scopes, Protein Purification (Springer-Verlag, N.Y., 1982)

Purification of human acid a-glucosidase from milk can be carried out by defatting of the transgenic milk by centrifugation and removal of the fat, followed by removal of caseins by high speed centrifugation followed by dead-end filtration (i.e., dead-end filtration by using successively declining filter sizes) or cross-flow filtration, or; removal of caseins directly by cross-flow filtration. Human acid a-glucosidase is purified by chromatography, including Q Sepharose FF (or other anion-exchange matrix), hydrophobic interaction chromatography (HIC), metal-chelating Sepharose, or lectins coupled to Sepharose (or other matrices).

Q Sepharose Fast Flow chromatography may be used to purify human acid a-glucosidase present in filtered whey or whey fraction as follows: a Q Sepharose Fast Flow (QFF; Pharmacia) chromatography (Pharmacia XK-50 column, 15 cm bed height; 250 cm/hr flow rate) the column was equilibrated in 20 mM sodiumphosphate buffer, pH 7.0 (buffer A); the S/D-incubated whey fraction (about 500 to 600 ml) is loaded and the column is washed with 4-6 column volumes (cv) of buffer A (20 mM sodium phosphate buffer, pH 7.0). The human acid a-glucosidase fraction is eluted from the Q FF column with 2-3 cv buffer A, containing 100 mM NaCl.

The Q FF Sepharose human acid a-glucosidase containing fraction can be further purified using Phenyl Sepharose High Performance chromatography. For example, 1 vol. of 1M ammonium sulphate is added to the Q FF Sepharose human acid aglucosidase eluate while stirring continuously. Phenyl HP (Pharmacia) column chromatography (Pharmacia XK-50 column, 15 cm bed height; 150 cm/hr flow rate) is then done at room temperature by equilibrating the column in 0.5 M ammonium sulphate, 50 mM sodiumphosphate buffer pH 6.0 (buffer C), loading the 0.5 M ammoniumsulphate-incubated human acid a-glucosidase eluate (from Q FF Sepharose), washing the column with 2-4 cv of buffer C, and eluting the human acid a-glucosidase was eluted from the Phenyl HP column with 3-5 cv buffer D (50 mM sodiumphosphate buffer at pH 6.0). Alternative methods and additional methods for further purifying human acid a-glucosidase will be apparent to those of skill. For example, see United Kingdom patent application 998 07464.4 (incorporated by reference in its entirety for all purposes).

The present invention provides inter alia methods of purifying heterologous proteins from the milk of transgenic animals, preferably human acid a-glucosidase. The methods are amenable for large-scale production, and result in proteins including a-glucosidase in a form suitable for therapeutic administration. The methods are particularly suitable for isolating human proteins and in particular human acid a-glucosidase from milk produced by transgenic animals. In one aspect the invention provides methods entailing two chromatography steps, one an anion-exchange column or affinity chromatography step, the other a hydrophophic interaction column or using hydroxylapatite in batch or column chromatography format. The two different separations act in a synergistic fashion substantially eliminating contaminating proteins present in a milk composition. For example, an anion exchange column separates human acid a-glucosidase from acid whey protein but not completely from serum albumin and transferrin. A hydrophobic interaction column effectively separates human acid a-glucosidase from serum albumin and transferrin but not from acid whey protein.

A typical purification procedure may involve addition steps before and after the above column purifications. For example, when human acid a-glucosidase is purified from milk, fat and caseins are removed from milk before column chromatography. The procedure can also include further steps to eliminate any viruses that may be present. a-Glucosidase is then separated from whey proteins and other milk proteins by the two column steps noted above. Each or both of these may be performed more than once until a desired degree of purification has been achieved. After column chromatography a-glucosidase is optionally concentrated and resuspended in a storage buffer.

In another aspect the invention provides a procedure involving hydroxylapatite under optimised conditions wherein the heterologous protein is substantially unable to bind to the matrix whereas the contaminating milk proteins are substantially bound. The method provides a quick and reproducible one step clean up giving a substantial purification of the heterologous protein of interest.

Sources of a-gucosidase

As noted, the methods are particularly suited to the purification of human acid a-glucosidase from the milk of transgenic animals. Production of a-glucosidase in the milk of transgenic animals is described by WO 97/05771 (incorporated by reference in its entirety for all purposes). Briefly, regulatory sequences from a mammary gland specific gene, such as a-s1-casein are operably linked to an a-glucosidase coding sequence. The transgene is then introduced into an embryo, which is allowed to develop into a transgenic mammals. Female transgenic mammals express the transgene in their mammary gland and secrete human acid a-glucosidase into milk. For mice, levels up to 4 gram per liter and for rabbit, levels up to 7 gram per liter can be obtained.

Transgenic rabbits are of particular interest since they breed fast, so a production herd can be established in a short time frame, and they produce significant quantities of milk (up to 0.5 liter/week) containing about 150 gram of protein per liter. Transgenic cows (DeBoer et al., WO 91/08216) are also of interest since they produce, at low costs, large quantities of milk (about 10,000 liters/year) containing about 35 gram of protein per liter [Swaisgood, Developments in Dairy Chemistry-1, Ed. Fox, Elsevier Applied Science Publisher, London (1982) Pp. 1-59]. Goats, sheep, pigs, mice and rats are also appropriate hosts for expression of a-glucosidase in their milk (see, e.g., Rosen, EP 279,582, Simon et al., BiolTechnology 6,179-183 (1988)).

Other sources of human acid a-glucosidase include cellular expression systems (e.g., bacterial, insect, yeast or mammalian) and natural sources, such as human tissues (e.g., liver from cadavers).

II. Defattina Milk

Defatting of the rabbit milk can be done using conventional methods e.g. low-speed centrifugation (about 2000 g) with a Hettich Rotanta R P, Sorvall RC-5B, or a continuous flow centrifugation appliance such as an Elecrem that result in a required efficiency of fat removal. Milk can be collected and frozen directly, or can first be defatted and then frozen. Optionally, separated fat can be washed with water or a low salt buffer, and the wash subsequently re-centrifuged to improve the recovery of product to be purified. Also other methods as used in the bovine dairy industry for fat removal can be applied (e.g. filtration).

III. Removal of Caseins from Milk

Caseins can be removed from milk by various methods. Some methods employ either acid treatment or heat shock. For example, in one method, skimmed milk is brought to pH 4.7, incubated for about 30 min, followed by e.g. centrifugation. Optionally, a temperature shock can be applied after adjusting to pH 4.7, from e.g. 10 C. to about 35 C., again followed by (low-speed ˜2000 g for a few minutes) centrifugation. Although this method can be employed in the separation of caseins from milk containing human acid a-glucosidase, it is not preferred because human acid a-glucosidase is sensitive to both pH and temperature treatment. Human acid a-glucosidase activity is in general significantly decreased when the pH drops below 4.5, or when the temperature is raised above 40 C.

Other methods of separating casein from milk use high-speed centrifugation and/or dead-end filtration and/or tangential flow filtration. Centrifugation can be performed on a large scale using a Powerfuge (hundreds of liters of skimmed milk) to remove caseins. Since the efficiency of casein removal is not 100% but more like 80-90%, the centrifuged whey is further clarified before subsequent chromatography. Clarification can be done by either dead-end filtration (i.e., use of filters of successively smaller pore size) or cross-flow filtration (i.e.TFF) can be used.

Tangential flow filtration gives the best results: clear whey is obtained with high acid a-glucosidase passage over the membrane (>90% recovery of product can be obtained after diafiltration). Tangential flow filtration (also known as cross flow filtration) is a special way of filtration that leads to less clogging of the membrane due to the recirculated flow transverse to the membrane. The advantage in a pharmaceutical (industrial) process is that these types of membranes can be reused after cleaning, in contrast to dead-end filters. As well as being used as a source of acid a-glucosidase for subsequent purification, whey resulting from TFF can be used to produce food products containing whey. Separated caseins can also be used in food production.

In the tangential flow filtration mode, several types of membranes have been tested. Various membranes were found to be suitable (meaning a clear filtrate with high acid a-glucosidase passage): pores varied from about 0.05 to 0.3 um with a preference for a pore size of 0.1 to 0.2 um. Processing of a Powerfuge whey fraction over a Biomax 1000k membrane (Millipore) yielded a clear whey filtrate with a passage of human acid a-glucosidase of 60-80%.

The recovery can be increased up to >97% after washing the retentate fraction with a buffer (e.g. 20 mM sodium phosphate buffer pH 7.0) in the so-called diafiltration mode.

Cross-flow filtration can be used to separate caseins from milk without a prior high speed centrifugation step. Clear whey is obtained with a passage of human acid a-glucosidase of 65-35%. With diafiltration (addition of buffer to maintain volume) the recovery can be increased to >90%. After diafiltration the filtrate has to be concentrated. This can be done easily with ultrafiltration using e.g. a Biomax 30k (Millipore) membrane or any other membrane with a pore-size so small that acid a-glucosidase does not pass the membrane.

IV. Column Chromatography

One preferred method according to the invention employs two column chromatography steps, one an anion exchange or affinity column, the other a hydrophobic interaction column. The steps can be performed in either order. Either or both of the steps can be repeated to obtain a higher degree of purity.

Anion exchange columns have two components, a matrix and a ligand. The matrix can be, for example, cellulose, dextrans, agarose or polystyrene. The ligand can be diethylaminoethyl (DEAE), polyethyleneimine (PEI) or a quaternary ammonium functional group. The strength of an anion exchange column refers to the state of ionization of the ligand. Strong anionic exchange columns, such as those having a quaternary ammonium ligand, bear a permanent positive charge over a wide pH range. In weak anion exchange columns, such as DEAE and PEI, the existence of the positive charge depends on the pH of the column. Strong anion exchange columns such as Q Sepharose FF, or metal-chelating Sepharose (e.g., Cu2+-chelating Sepharose) are preferred. Anion exchange columns are generally loaded with a low-salt buffer at a pH above the pl of a-glucosidase.

The calculated p1 of a-glucosidase is 5.4 (SWISS-PROT database). The columns are washed several times in the low-salt buffer to elute proteins that do not bind. Proteins that have bound are then eluted using a buffer of increased salt concentration.

Q Sepharose FF is a preferred anion exchange column because this material is relatively inexpensive compared with other anion-exchange columns and has a relatively large bead size suitable for large scale purification. Under specified conditions Q Sepharose FF binds human acid a-glucosidase and separates a-glucosidase sufficiently from the strongest binding (milk) proteins. This is essential since some of these strongly binding proteins, for instance rabbit whey acidic protein (WAP), tend to co-elute with a-glucosidase in the subsequent hydrophobic interaction chromatography (HIC) steps. To obtain good binding of human acid aglucosidase to the Q Sepharose FF, the column is pre-equilibrated in low salt (i.e., less than 50 mM, preferably less than 35 mM such as sodium or potassium phosphate buffer or other suitable salts such as Tris. The pH of the buffer should be about 7.0+/−1.0 to obtain a good binding of human acid a-glucosidase to the column. A much higher pH is not suitable because human acid a-glucosidase is inactivated to some extent. A much lower pH weakens binding of a-glucosidase to the anion-exchange material.

Human acid a-glucosidase is then eluted by step-wise or gradient elution at increased salt concentration. Step-wise elution is more amenable to largescale purification. About 85% of loaded human acid a-glucosidase can be eluted from a Q FF column in one step (at about 0.1 M salt) with relatively high purity. The main protein contaminants when a-glucosidase is purified from rabbit milk are rabbit milk-derived proteins like transferrin and serum albumin. Strongly binding milk proteins, such as WAP, elute from Q Sepharose FF with higher salt concentrations, e.g. about 1 M salt.

Optionally, the anion-exchange step can be replace with an affinity chromatography step, although such is not preferred. Suitable affinity reagents include lectins and antibodies. Lectins are plant-derived carbohydrate binding proteins that have affinity for glycoproteins. Proteins are typically loaded on lectin columns in a buffer of about 150 mM salt and neutral pH containing about 1 mM Ca2+ or Mg2+. Glycoproteins can be eluted from such columns using a buffer containing 0.1-0.5 M concentration of a simple sugar, such as sucrose. Examples of lectin affinity columns includes lectins coupled to Sepharose (or other matrices) such as lentil Sepharose (reported to be less toxic compared to Concanavalin A). Also, ligands recognizing vicinal diols can be used, such as (amino) phenyl boronate. Monoclonal or polyclonal antibodies to human acid a-glucosidase can also be used as affinity reagents. Antibodies are typically linked to cyanogen bromide activated Sepharose. Non-specifically bound or weakly bound proteins can be eluted from such a column using a neutral buffer at moderately high salt concentration (i.e., greater than about 0.5 M).

Specifically bound (x-glucosidase is the eluted using low pH buffer (e.g., 50 mM citrate, pH 3.0). Following elution, a-glucosidase should be neutralized.

Antibody-based affinity purification is not preferred relative to anion exchange, because antibodies are relatively expense reagents, and as a biologic are subject to FDA review if the ultimate goal of purification is to produce a protein for therapeutic use.

The second column used for isolating human acid a-glucosidase is a hydrophobic interaction chromatography (HIC) column. HIC columns have two components, a matrix and a ligand. Suitable matrices include Sepharose and polystyrene. Suitable ligands include phenyl-, butyl-, octyl-, and ether-groups. Phenyl-Sepharose™ or (Source Phenyl 15 (phenyl group linked to polystyrene column)) are particularly suitable. The loading buffer for HIC chromatography contains a high concentration of a salt that favours hydrophobic interactions. Suitable anions are phosphate, sulphate and acetate. Suitable cations are ammonium, rubidium and potassium. For example, a solution of about 0.5+/−0.2 M ammonium sulphate, pH 6 is suitable. Under these conditions, human acid a-glucosidase binds to the column whereas most other proteins do not. a-glucosidase can then be eluted with a low salt elution buffer. For example, buffer of 25-100 mM, preferably 50 mM sodium phosphate buffer, pH about 6.0 (+/−1.0) is suitable).

The relative order of elution of human acid a-glucosidase and other milk proteins depends on the nature of the column For example, on a phenyl-Sepharose column, a-glucosidase binds better than serum albumin. On a (Source Phenyl 15) column the reverse is the case. Transferrin binds more weakly to a Source Phenyl 15 column and a phenyl-Sepharose column.

Transferrin binding can be blocked at (e.g. 0.5 M ammonium sulfate).

V. Viral Elimination

For the removal of viruses, a solvent/detergent step can be incorporated at any point in the procedure, usually after removal of fat and caseins from milk. A specific combination of solvent and detergent, like 0.3% tri-n-butylphosphate (TnBP) combined with 1% Tween-80, is very effective in the removal of enveloped viruses (Horowitz et al (1985) Transfusion 25, pp. 516-522). A whey fraction obtained after cross-flow filtration was incubated for 6 hours at 25 C with 0.3% TnBP and 1% Tween-80. After this incubation, the whey was directly loaded on a Q FF chromatography column.

Uses of Purified Human Acid a-Glucosidase

Purified human acid a-glucosidase produced according to the invention finds use in enzyme replacement therapeutic procedures. A patient having a genetic or other deficiency resulting in an insufficiency of enzyme can be treated by administering exogenous enzyme to the patient. Patients in need of such treatment can be identified from symptoms (e.g., cardiomegaly, hepatosplenomegaly, increased numbers of lysosomes and markers thereof, joint stiffness). Alternatively, or additionally, patients can be diagnosed from biochemical analysis of a tissue sample to reveal excessive accumulation of a metabolite processed by a-glucosidase or by enzyme assay using an artificial or natural substrate to reveal deficiency of acid a-glucosidase.

Diagnosis can be made by measuring the particular enzyme deficiency or by DNA analysis before occurrence of symptoms or excessive accumulation of metabolites (Scriver et al., supra, chapters on lysosomal storage disorders). a-Glucosidase storage diseases are hereditary. Thus, in offspring from families known to have members suffering from a-glucosidase, it is sometimes advisable to commence prophylactic treatment even before a definitive diagnosis can be made.

In some methods, human acid a-glucosidase is administered in purified form together with a pharmaceutical carrier as a pharmaceutical composition.

The preferred form depends on the intended mode of administration and therapeutic application. The pharmaceutical carrier can be any compatible, nontoxic substance suitable to deliver the polypeptides to the patient. Sterile water, alcool, fats, waxes, and inert solids can be used as the carrier.

Pharmaceutically-acceptable adjuvants, buffering agents, dispersing agents, and the like, may also be incorporated into the pharmaceutical compositions.

The concentration of the enzyme in the pharmaceutical composition can vary widely, i.e., from less than about 0.1% by weight, usually being at least about 1% by weight to as much as 20% by weight or more.

For oral administration, the active ingredient can be administered in solid dosage forms, such as capsules, tablets, and powders, or in liquid dosage forms, such as elixirs, syrups, and suspensions. Active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate and the like. Examples of addition inactive ingredients that may be added to provide desirable color, taste, stability, buffering capacity, dispersion or other known desirable features are red iron oxide, silica gel, sodium lauryl sulfate, titanium dioxide, edible white ink and the like. Similar diluents can be used to make compresse tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain colouring and flavouring to increase patient acceptance.

A typical composition for intravenous infusion could be made up to contain 100 to 500 ml of sterile 0.9% NaCl or 5% glucose optionally supplemented with a 20% albumin solution and 100 to 500 mg of enzyme. A typical pharmaceutical compositions for intramuscular injection would be made up to contain, for example, 1 ml of sterile buffered water and 1 to 10 mg of the purified enzyme of the present invention. Methods for preparing parenterally administrable compositions are described in more detail in various sources, including, for example, Remington's Pharmaceutical Science (15th ed., Mack Publishing, Easton, Pa., 1980) (incorporated by reference in its entirety for all purposes).

The pharmaceutical compositions of the present invention are usually administered intravenously. Intradermal, intramuscular or oral administration is also possible in some circumstances. The compositions can be administered for prophylactic treatment of individuals suffering from, or at risk of, a lysosomal enzyme deficiency disease. For therapeutic applications, the pharmaceutical compositions are administered to a patient suffering from established disease in an amount sufficient to reduce the concentration of accumulated metabolite and/or prevent or arrest further accumulation of metabolite. For individuals at risk of lysosomal enzyme deficiency disease, the pharmaceutical composition are administered prophylactically in an amount sufficient to either prevent or inhibit accumulation of metabolite. An amount adequate to accomplish this is defined as a “therapeutically-” or “prophylactically-effective dose.” Such effective dosages will depend on the severity of the condition and on the general state of the patient's health, but will generally range from about 0.1 to 10 mg of purified enzyme per kilogram of body weight.

Human acid a-glucosidase produced in the milk of transgenic animals has a number of other uses. For example, a-glucosidase, in common with other a-amylases, is an important tool in production of starch, beer and pharmaceuticals. See Vihinen & Mantsala, Crit. Rev. Biochem. Mol. Biol. 24, 329-401 (1989) (incorporated by reference in its entirety for all purpose).

Human acid a-glucosidase is also useful for producing laboratory chemicals or food products. For example, acid a-glucosidase degrades 1,4 and 1,6 aglucosidic bonds and can be used for the degradation of various carbohydrates containing these bonds, such as maltose, isomaltose, starch and glycogen, to yield glucose. Acid a-glucosidase is also useful for administration to patients with an intestinal maltase or isomaltase deficiency.

Symptoms otherwise resulting from the presence of undigested maltose are avoided. In such applications, the enzyme can be administered without prior fractionation from milk, as a food product derived from such milk (e.g., ice cream or cheese) or as a pharmaceutical composition. Purified recombinant lysosomal enzymes are also useful for inclusion as controls in diagnostic kits for assay of unknown quantities of such enzymes in tissue samples.

### EXAMPLES

1. Materials and Methods

Acid a-ctlucosidase Assay

A 96-well microtiter plate (NUNC) was put on ice. and 20 ut 4-MU substrate (4-methyl umbelliferyl-a-D-glucopyranoside; Mellford Labs, London; 2.2 mM in 0.2 M Na Acetate buffer pH 4.3) was added in a well. Sample to be tested (10 ul, diluted in PBS (phosphate buffered saline)+0.5% BSA (w/v; Sigma fraction V)), was added and incubated for 30 min at 37 C. The reaction was stopped with 200 NI 0.5 M Na-carbonate buffer (pH 10.5). The microtiter plate was assayed in a fluorometer (excitation wavelength=360 nm; emission wavelength=460 nm). As a standard recombinant human mature acid aglucosidase was included in each assay.

Radio-iodination of Acid a-glucosidase

Recombinant human precursor acid a-glucosidase purified from transgenic rabbit milk (line 60) was radio-iodinated with the Chloramin T method. Labeling was essentially done as follows: to 0.2 ml of precursor (−0.1 mg) 10 NI of Na1251 (˜1 mCi) was added. Chloramin T (50 pl; 0.4 mg/ml in PBS) was added, and incubated for 60 seconds. Then, 50 ut Na2S205 (1 mg/ml in PBS) and 100 ul of a 0.2 mg/ml Na1solution in PBS was added. Free 1251 was separated on a PD 10 gel filtration column (Pharmacia) equilibrated in PBS, 0.1% Tween-20, 1 M NaCl, 0.05% sodium azide. Labeled protein was pooled and kept at −80 C.

Radio-assay with metal-chelating Sepharoses and lectin Sepharoses The binding of radio-iodinated precursor acid a-glucosidase to metal-chelating sepharose was measured to determine whether a specific metal interacts with the (radio-labeled) enzyme. Also the binding to lectin Sepharoses was determined.

Chelating Sepharose (Pharmacia) was incubated with various salts according to the recommendations of the manufacturer. Essentially the Sepharoses were prepared as follows: 3.5 ml packed Sepharose beads were diluted in 500 ml water, centrifuged (3500 rpm, 10 minutes), and after removal of the supernatant, the beads were resuspended in either 50 ml Cucu12 (257 mg), ZnCl2 (215 mg), ferric-sulphate (400 mg), or ferrous-sulphate (417 mg). After overnight incubation (rotating), the beads were washed 3 times with water, and then washed with PBS, 0.1% Tween-20, 1 M NaCI, and stored in 50 ml water at 4 C. For the binding experiment, 0.5 ml of the Sepharose beads were washed 5 times with PBS, 0.02% Tween-20, or PBS, 0.1% Tween-20, 0.5 M NaCI. Radio-labeled precursor enzyme (50 pi in PBS, 0.1% cpm) was added to 0.5 ml beads suspension, and incubated (rotating) overnight at room temperature. Sepharose beads were washed 4 times with PBS, 0.02% Tween-20, and the amount of bound label was counted in a liquid scintillation counter.

2. Skimmina (Defatting) of the Transgenic Milk

(0136) A. Milk was thawed in water bath at 25 C. while shaking. Then the milk was diluted 2-fold in water to maximize the recovery of the target protein and put into centrifugation bottles or tubes. The milk was defatted by centrifugation at 2800×g, for 15-30 min. at 4 C. The fat was removed with a spoon or by means of suction. Also full (undiluted) milk was centrifuged under the same conditions. The fat fraction obtained was: (1) washed with water and re-centrifuged, or (2) another batch was washed with a low salt buffer, and re-centrifuged. The skimmed milk and the wash fraction (after re-centrifugation) were pooled for further processing.

B. Milk was thawed in water bath at 25 C. while shaking, then the milk was put into an Elecrem centrifuge, using continuous centrifugation. The fat fraction was recovered, diluted in water, and re-centrifuged to maximize the recovery of human acid a-glucosidase in the pooled skimmed milk. A recovery of >90% can be obtained.

3. Preparation of the Whey Fraction from Skimmed Transgenic Milk Using Centrifugation and Dead-end Filtration

The removal of caseins from (diluted) skimmed rabbit milk was obtained by continuous centrifugation, at 20,000×g, for 30-45 min at 5-20 C. in a Powerfuge (Carr). The resulting whey fraction was made suitable for chromatography by dead-end filtration.

Dead-end filtration: first a CP15 or AP15 prefilter (Millipore) was used, followed by subsequent filtration over 1.2 pm RA, 0.8 pm AA, 0.65 pm DA and 0.45 um HA membrane filters (Millipore, disc-filters with a diameter of 47 mm) at a mild under-pressure. When clogging of filters occurred, new filters were used. The filtrate obtained after 0.45 pm membrane filtration was suitable for chromatography. The recovery of the target protein after centrifugation with the Carr Powerfuge was in general about 60-80%. Dead-end filtration resulted in a minimal loss of human acid a-glucosidase activity, in general <3%.

4. Preparation of the Whey Fraction from Skimmed Transgenic Milk Using TFF

Whey was prepared out of about 4.5 liter of diluted skimmed rabbit milk by TFF. A Biomax 1000 (0.5 m2) membrane cassette was placed in a cassette holder connected to a Proflux MA from Millipore. This membrane was chosen because it gives a very good retention of casein micelles (meaning the filtrate is very clear) and a passage of human acid a-glucosidase of about 30-60%. The process conditions were as follows: P-inlet=1.0 bar, P-outlet=0.7 bar, P-filtrate=0.7 bar, TransMembrane Pressure (TMP)=0.15 bar, flux=˜15 L/hr/m2, process temperature=10-35 C., preferably about 20 C. (room temperature). To improve the recovery of human acid a-glucosidase in the filtrate, the retentate was diluted with a low salt buffer, e.g. 20 mM sodium phosphate buffer at a pH of 7.0. After about 6 diafiltration volumes, the recovery of human acid a-glucosidase activity in the filtrate was >80%. Due to the diafiltration, the volume of the whey fraction (=filtrate) had increased dramatically. The filtrate was concentrated about 7 times by ultrafiltration using a Biomax 30 membrane (Millipore; 0.5 m2) in the same TFF device. This type of membrane is impermeable to a-glucosidase. A flux of 50 L/hr/m2 can easily be obtained in this step. The TMP was 1.0 bar. No activity was detected in the filtrate, but all activity was recovered in the retentate fraction. If the permeate contains to much sodium chloride, diafiltration was done with 20 mM sodium phosphate pH 7.0 buffer, to decrease the sodium chloride concentration below 5 mM.

5. Virus Inactivation by Solvent/Detergent (S/D)

Virus inactivation (at least of enveloped viruses) of the whey fraction was obtained by incubating the whey in the presence of 1% Tween-80 and 0.3% tri-n-butylphosphate (TnBP) while stirring continuously and mildly, for 4-8 hr (preferably 6 hr) at 25 C. No significant loss of a-glucosidase activity was observed (<10%).

6. Binding of Human Acid a-glucosidase Present in Filtered Whey or Whey Fraction to Q Sepharose

Fast Flow Q Sepharose Fast Flow (QFF; Pharmacia) chromatography (Pharmacia XK-50 column, 15 cm bed height; 250 cm/hr flow rate; all column chromatography controlled by the AKTA system of Pharmacia; protein was detected on-line by measuring the absorbance at 280 nm) was done using the following protocol:

1. the column was equilibrated in 20 mM sodium phosphate buffer, pH 7.0 (buffer A).

2. the S/D-incubated whey fraction (about 500 to 600 ml) was loaded.

3. after loading the whey fraction, the column was washed with 7 column volumes (cv) of buffer A.

4. the human acid a-glucosidase fraction was eluted from the Q FF column with 3.5 cv buffer A, containing 100 mM sodium chloride.

5. all strongly bound proteins were eluted with about 3 cv 100% buffer B, containing 1 M sodium chloride in 20 mM sodium phosphate buffer pH 7.0.

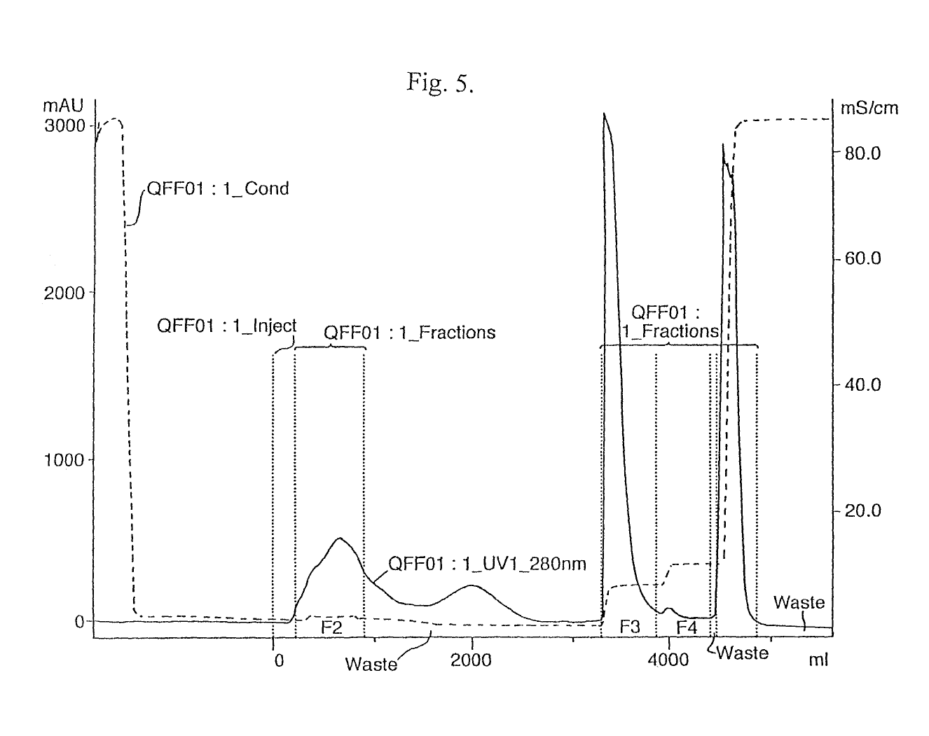

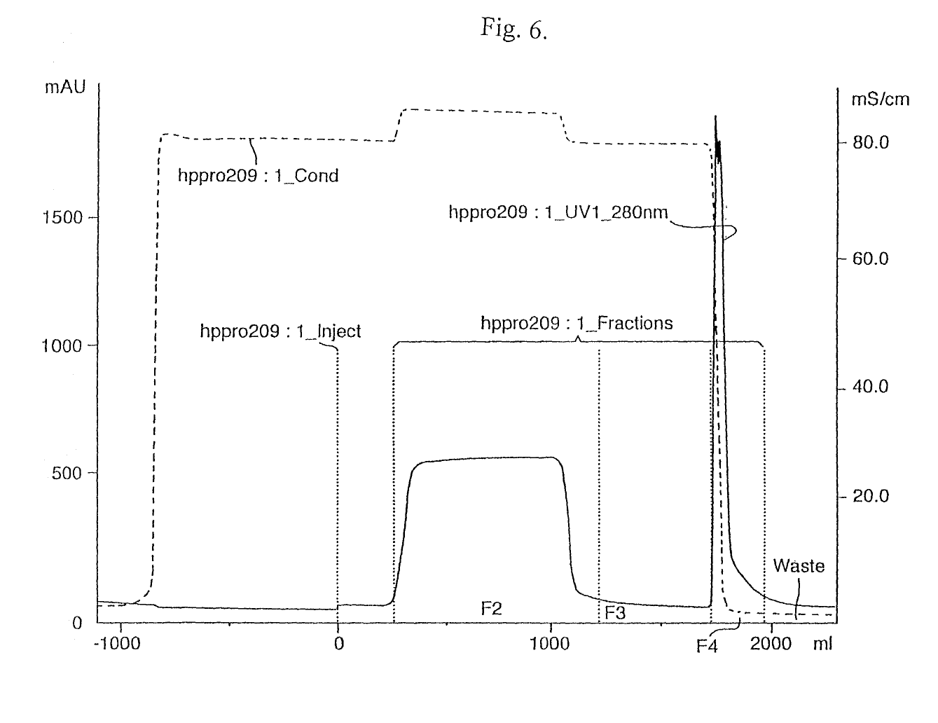

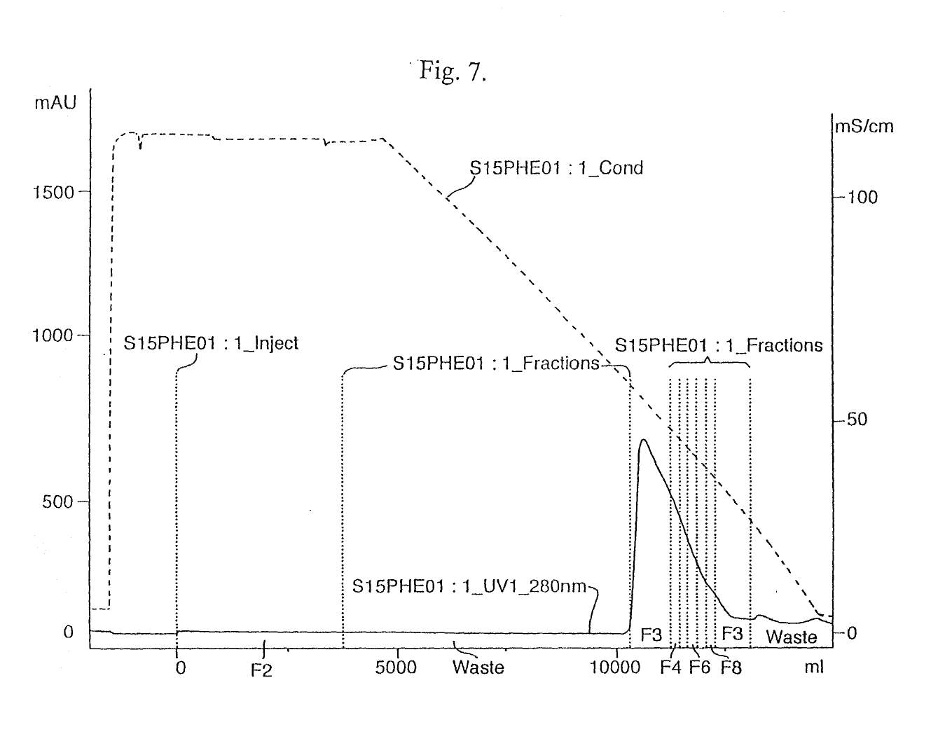

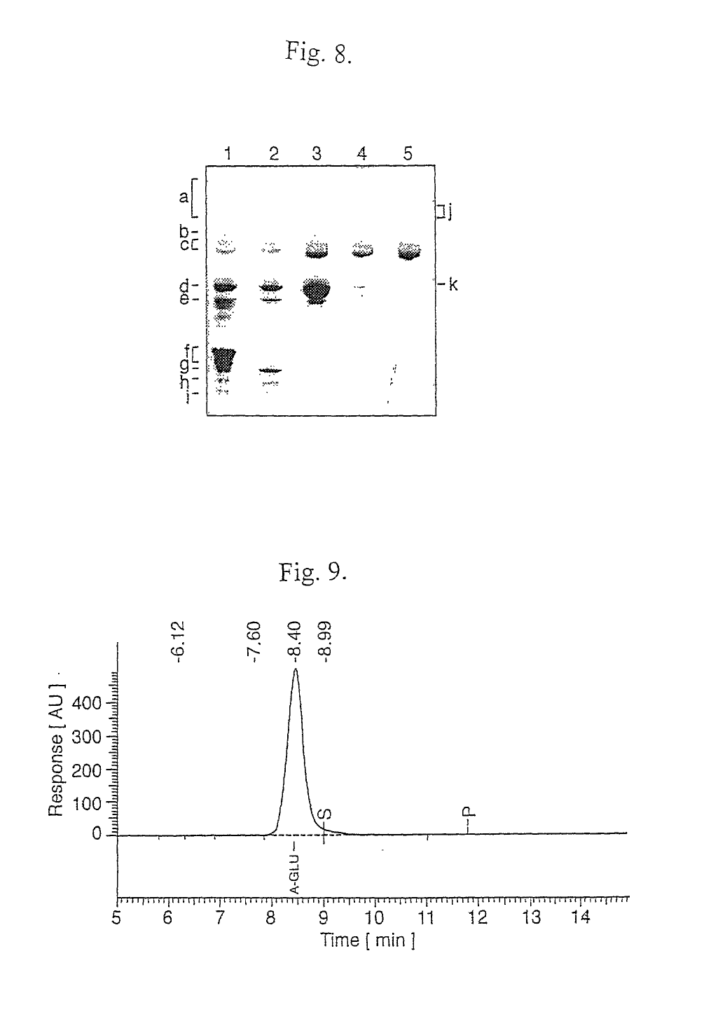

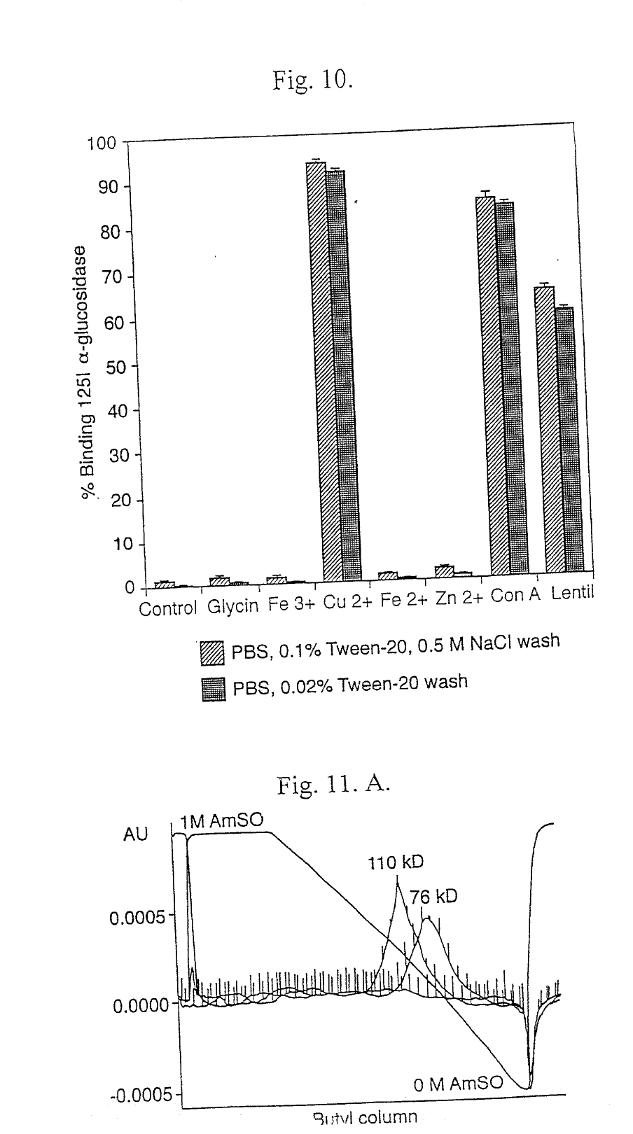

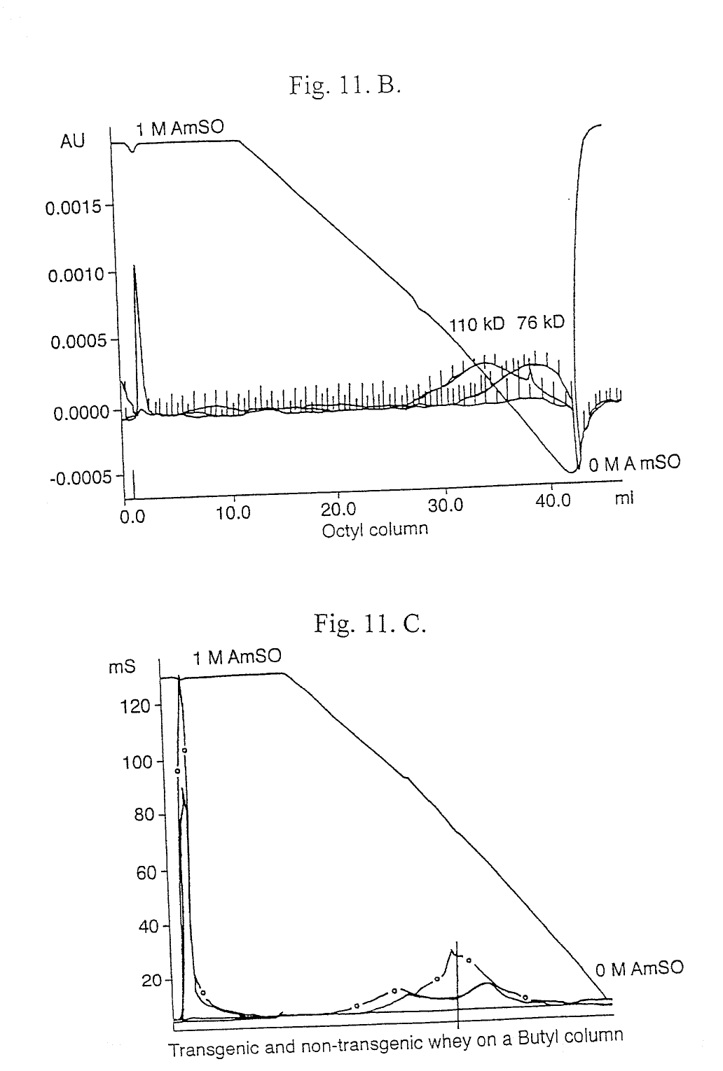

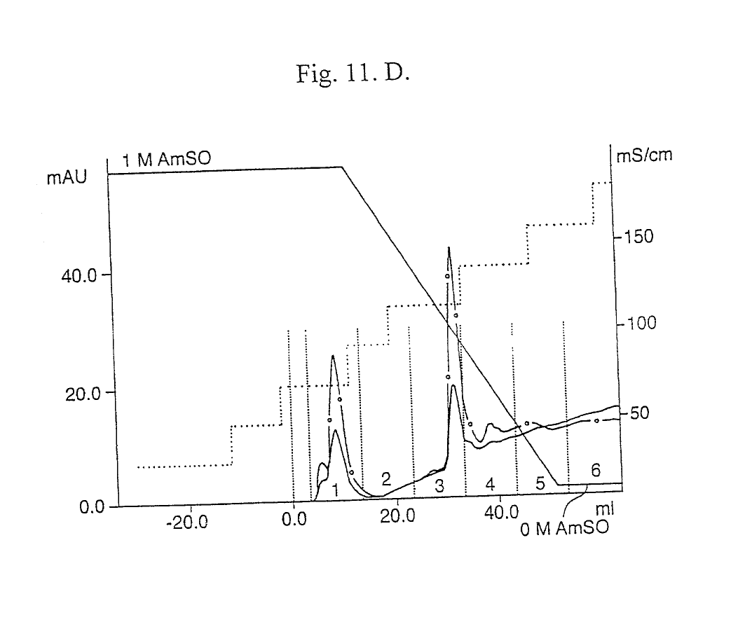

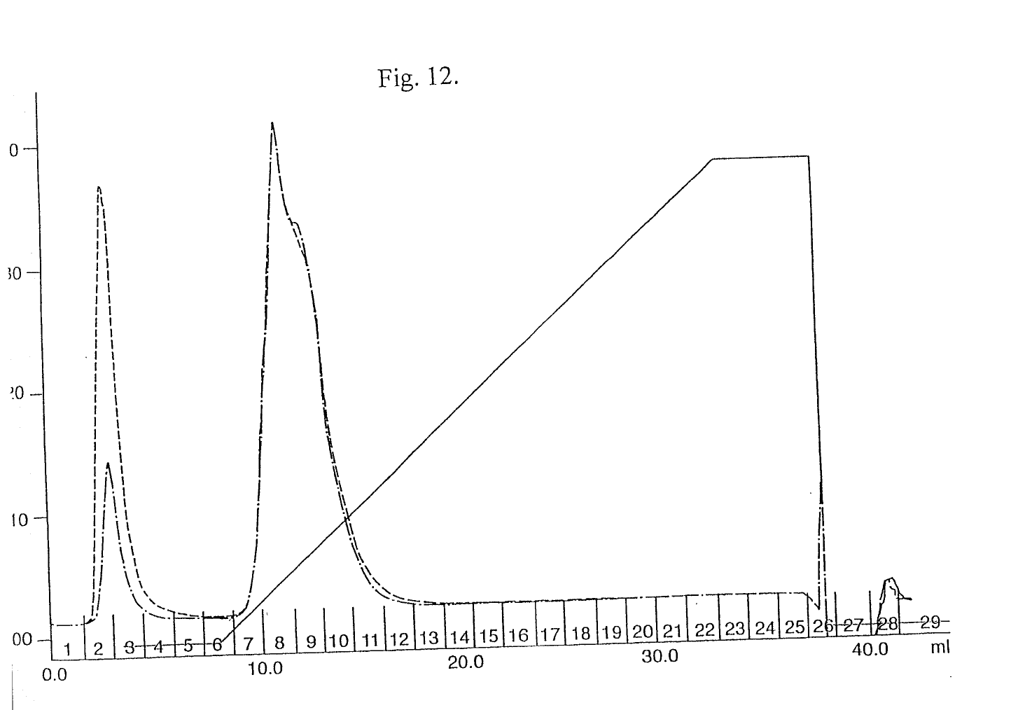

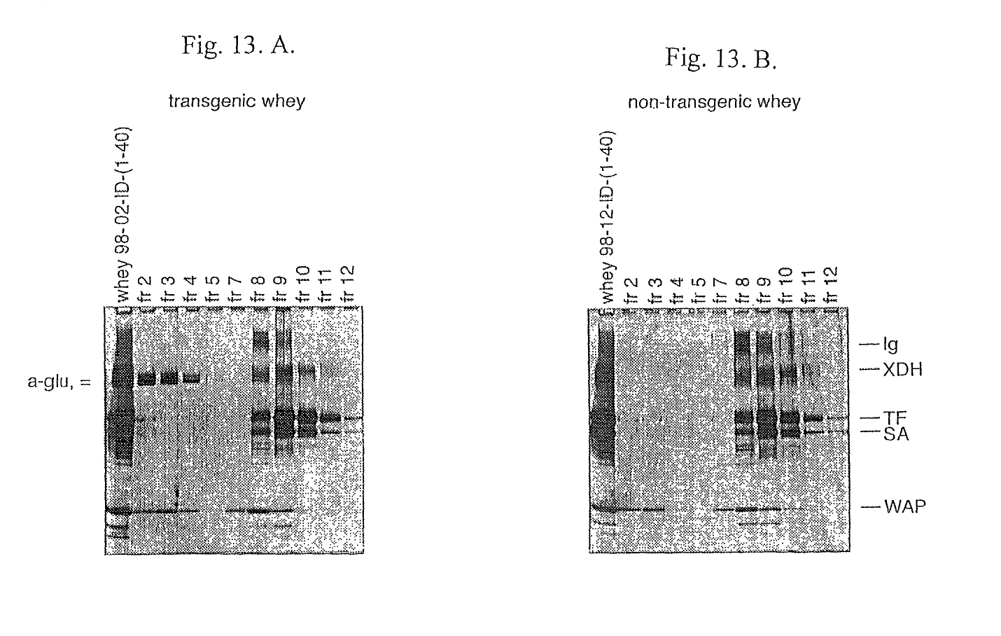

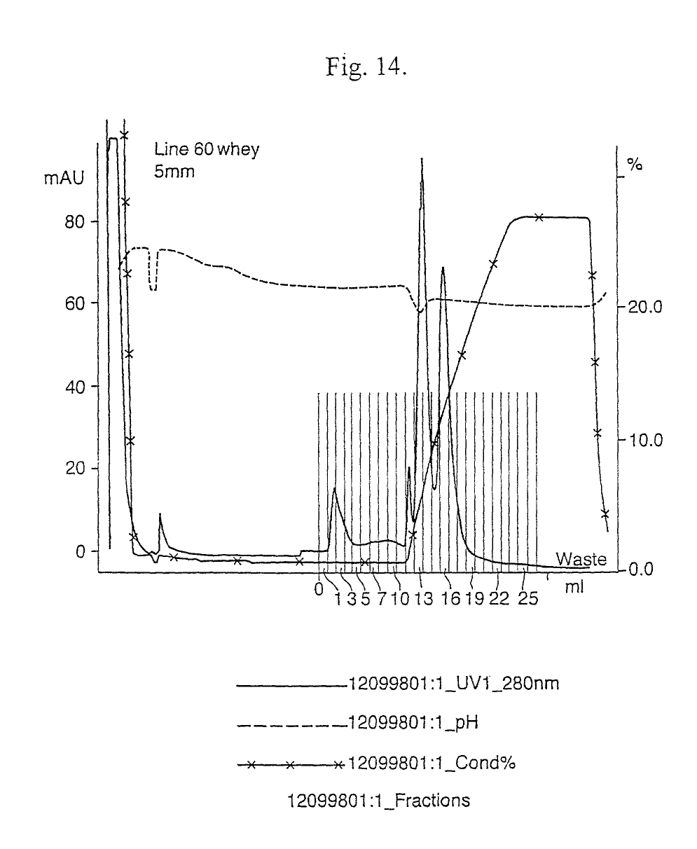

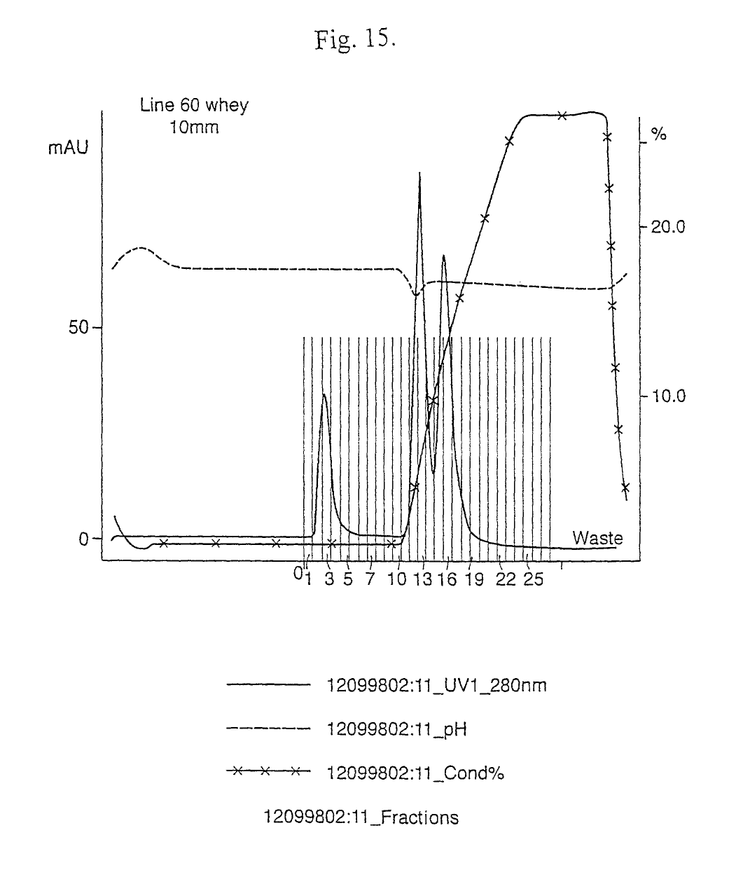

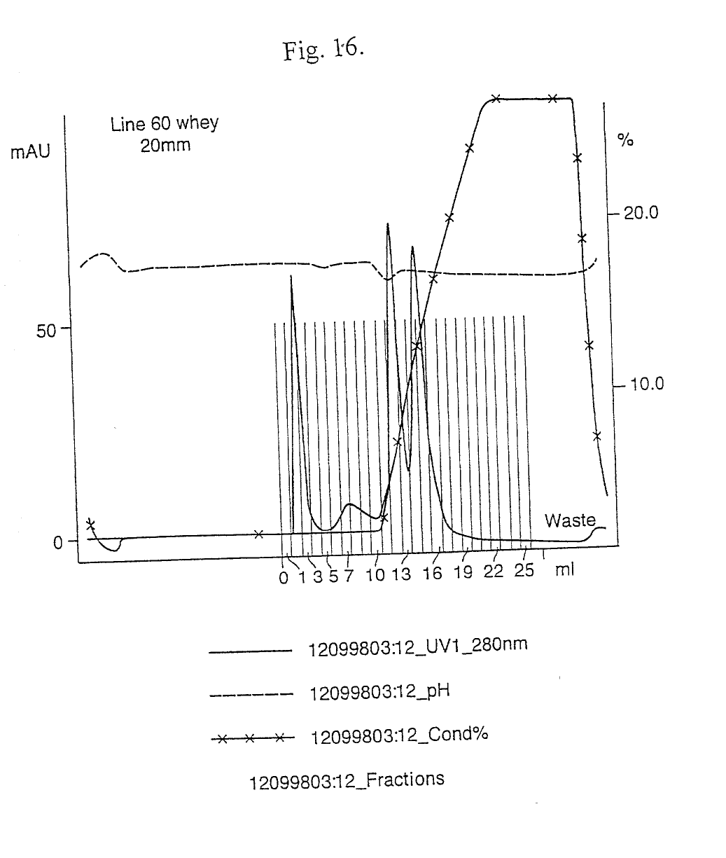

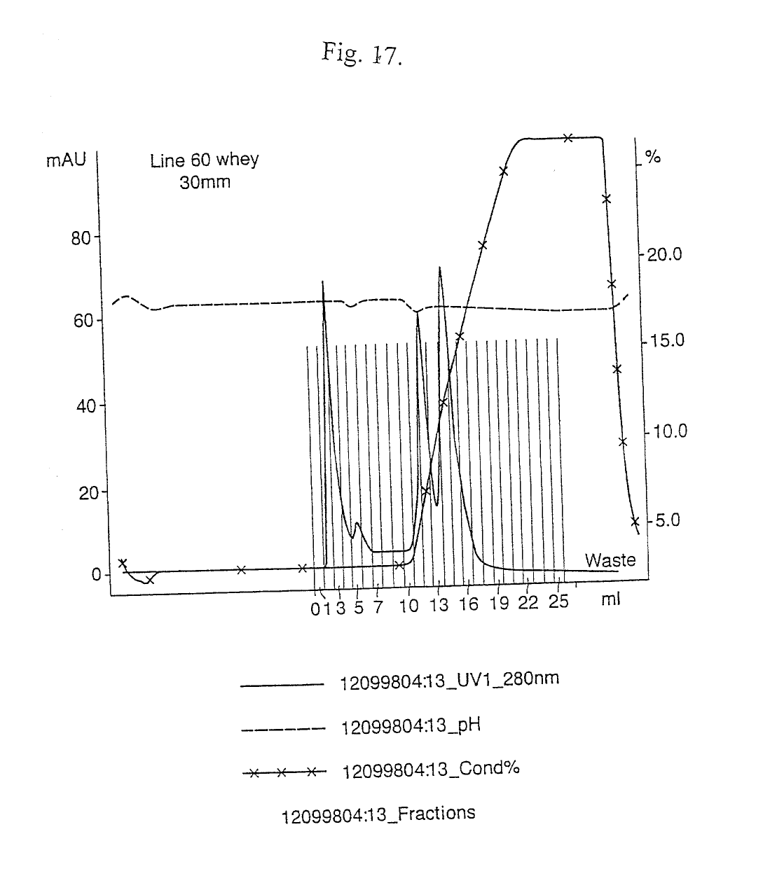

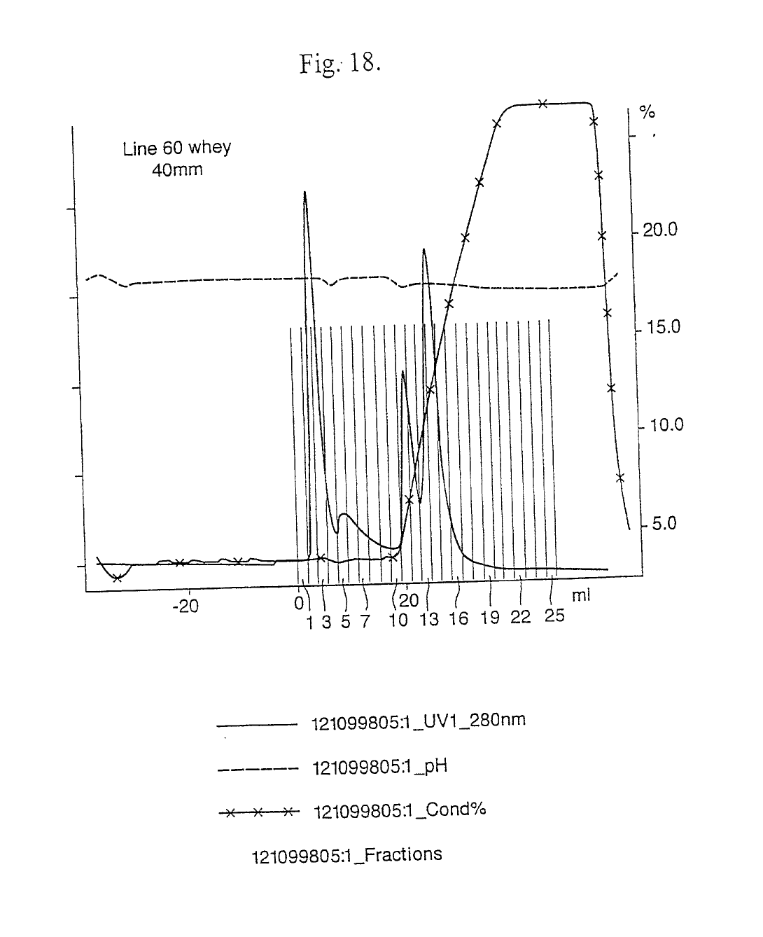

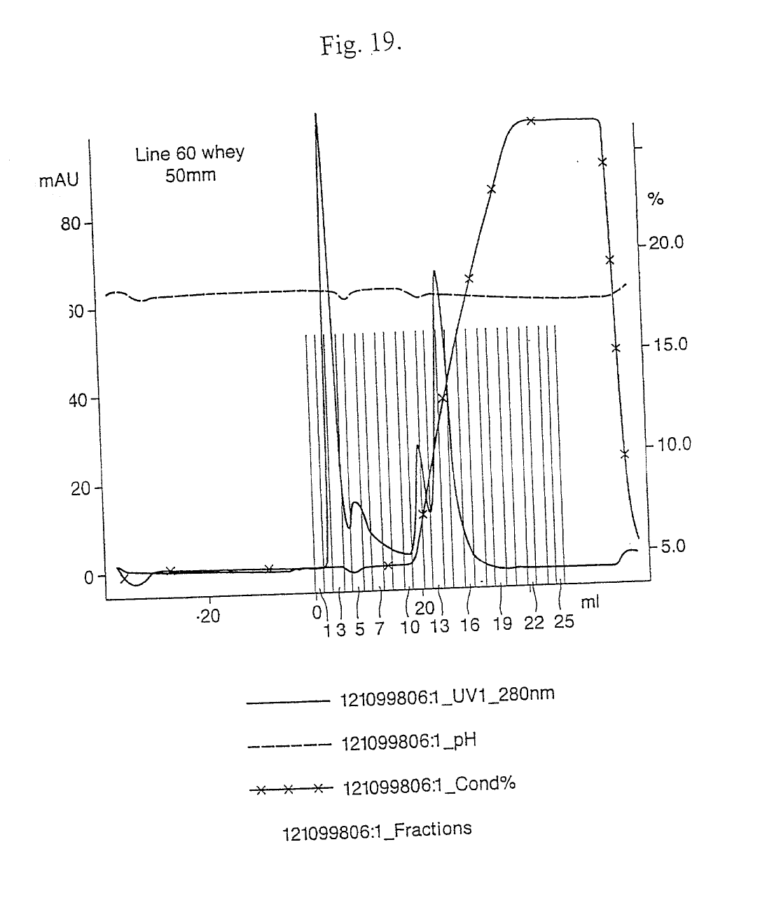

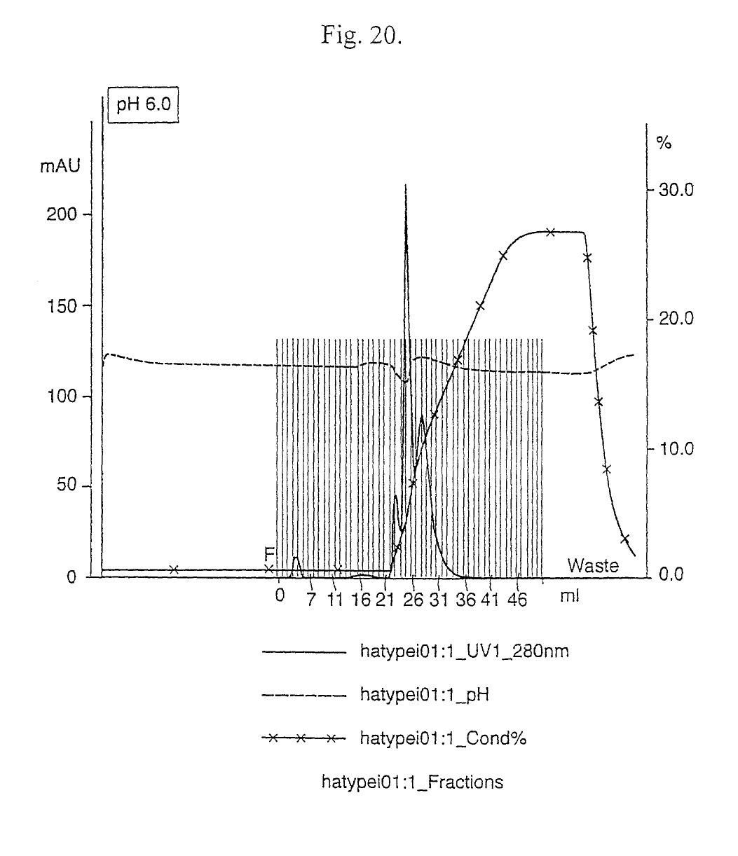

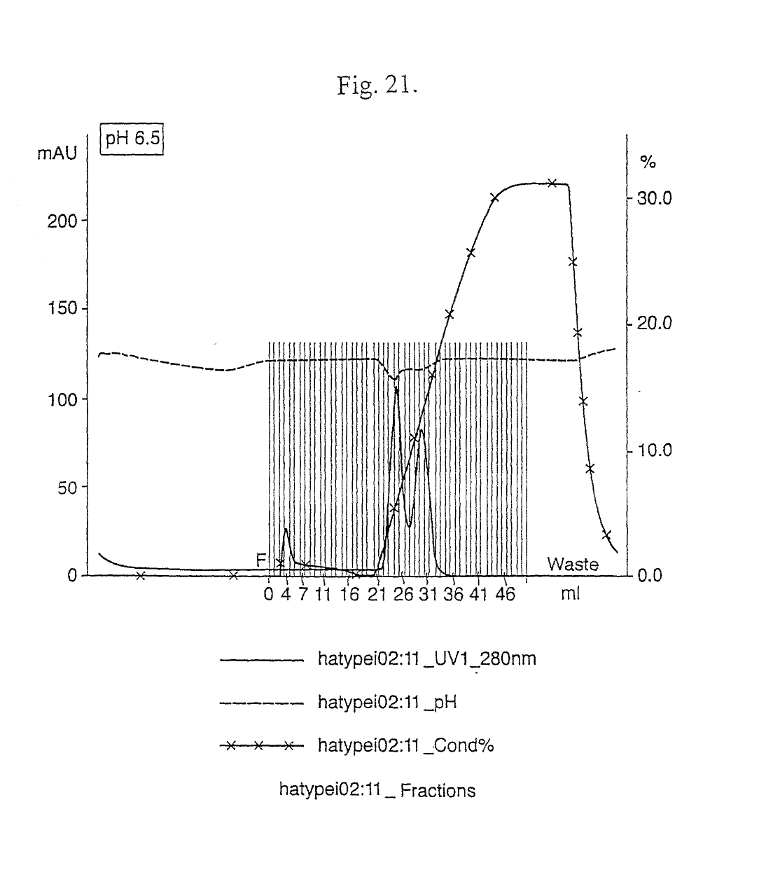

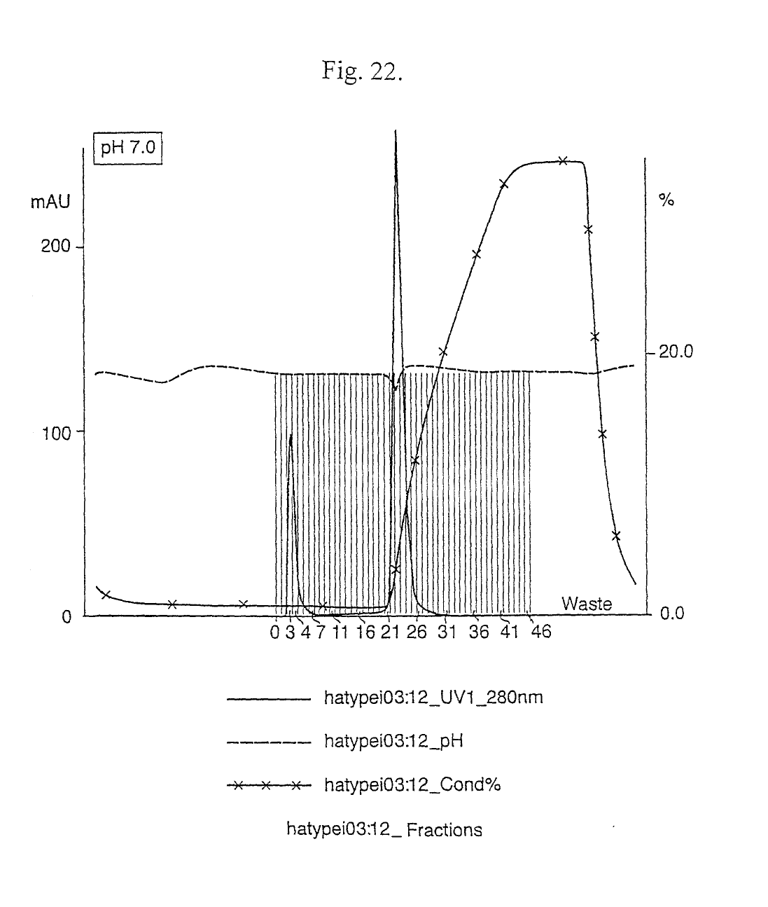

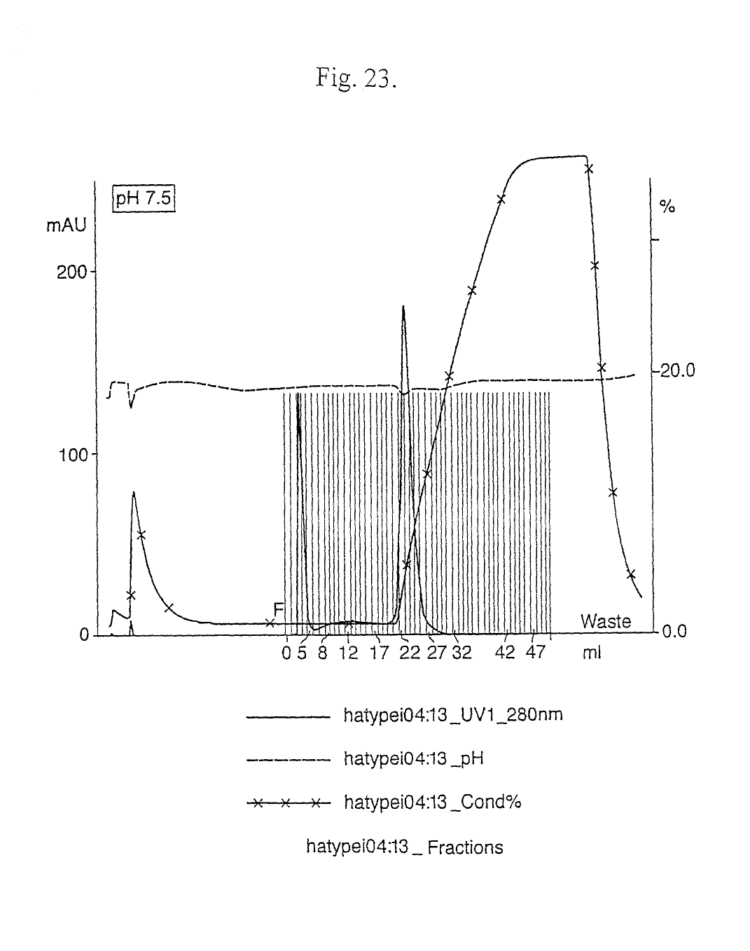

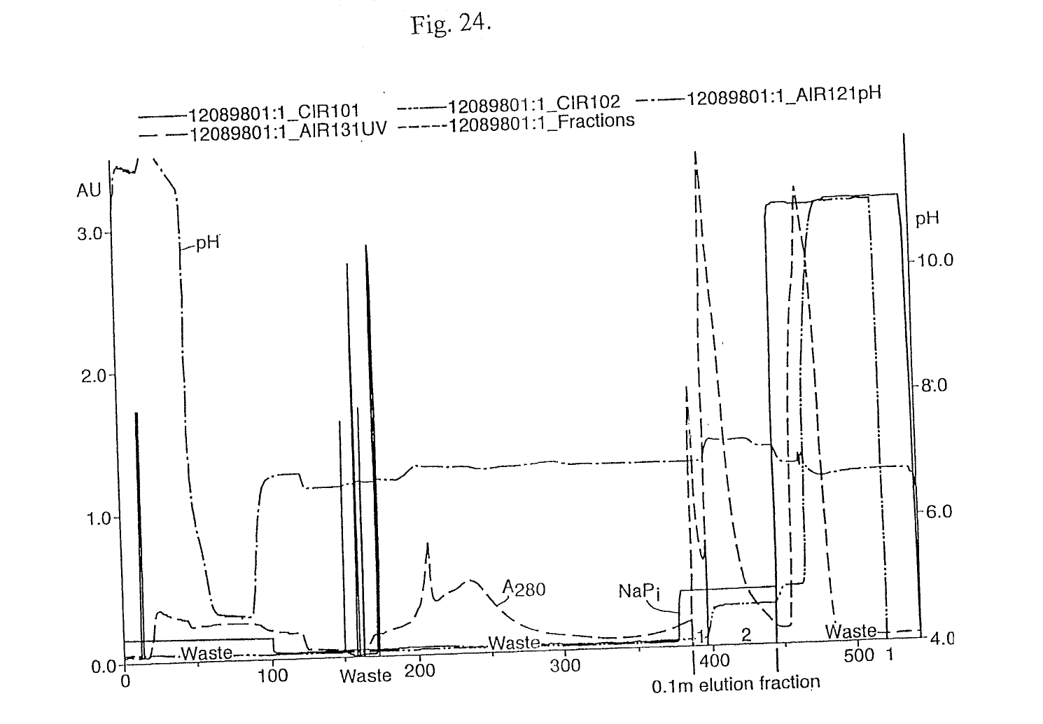

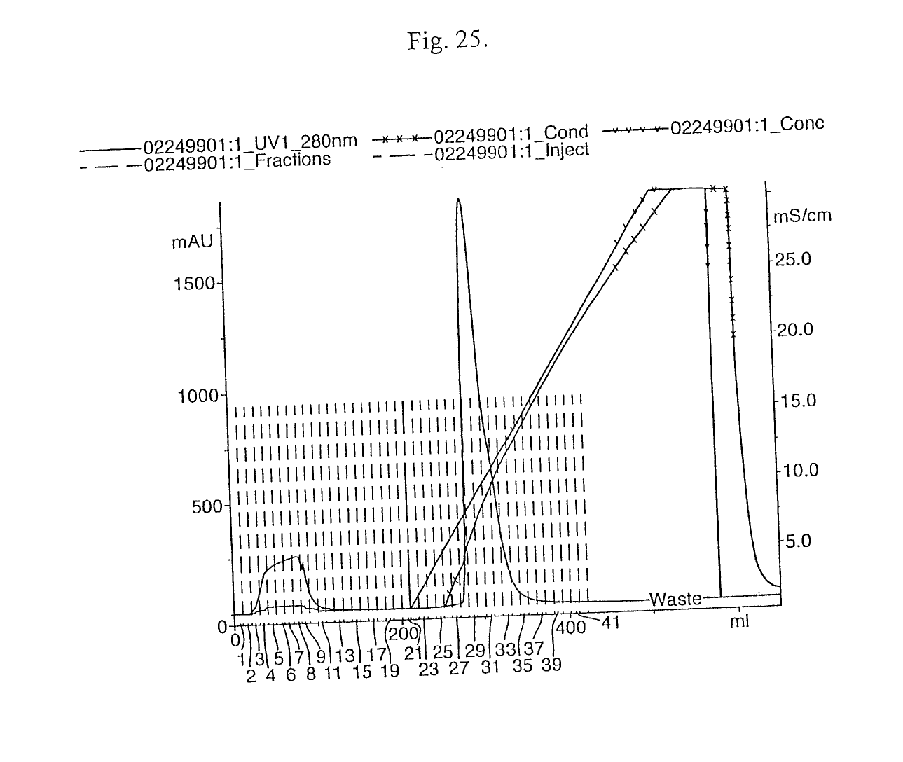

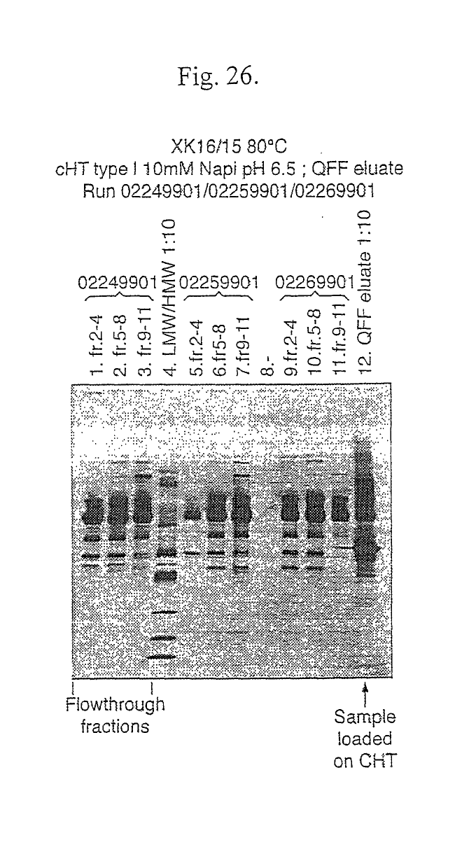

A representative elution profile of a Q FF chromatography run is shown in FIG. 5. In this specific run, the whey sample loaded on th Q FF column was S/D pretreated. Essentially the same elution profiles were obtained in a whey fraction, which was not subjected to S/D treatment, was loaded on the Q FF column No Tween-80 or TnBP could be detected in the recombinant human acid a-glucosidase fraction eluting in buffer A containing 100 mM sodium chloride. Essentially all Tween-80 and TnBP could be detected in the (unbound) flow through fraction. The recovery of recombinant human acid a-glucosidase (Step 4) was about 80-85%. About 15% of the aglucosidase activity was present in the fraction eluting with 100% buffer B.

7. Binding of Q FF Sepharose Human Acid a-Glucosidase Containing Fraction to Phenyl-SePharose High Performance

One volume of 1 M ammonium sulphate was added to the Q FF Sepharose human acid a-glucosidase eluate containing the major human acid a-glucosidase fraction (obtained with 0.1 M sodium chloride, 20 mM sodium phosphate buffer pH 7.0; see Example 10) while stirring continuously. Phenyl HP (Pharmacia) column chromatography (Pharmacia XK-50 column, 14 cm bed height; 150 cm/hr flow rate) was done at room temperature using the following protocol:

1. the column was equilibrated in 0.5 M ammonium sulphate, 50 mM sodium phosphate buffer pH 6.0 (buffer C).

2. the 0.5 M ammonium sulphate-incubated human acid a-glucosidase eluate was loaded. The dynamic capacity was about 1.2 mg human acid aglucosidase/ml Phenyl Sepharose High Performance.

3. after loading the sample, the column was washed with 2 cv of buffer C.

4. most human acid a-glucosidase was eluted from the Phenyl HP column with 4 cv buffer D (50 mM sodium phosphate buffer at pH 6.0).

5. the strongest binding proteins were eluted first with water, and then with 20% ethanol.

A representative elution profile of a Phenyl Sepharose HP chromatography run is shown in FIG. 6. The recovery of human acid a-glucosidase activity in step 4 was generally >85%.

8. Binding and Elution of Human Acid a-Glucosidase Fraction from the Phenyl HP Column on Source Phenyl 15

A 2 M ammonium-sulphate, 50 mM sodium phosphate buffer, pH 7.0 was added to the human acid a-glucosidase eluate from the Phenyl HP column, until a final concentration of 0.85 M ammonium sulphate was reached. The solution was stirred continuously and mildly.

Source Phenyl 15 (Pharmacia) chromatography (Pharmacia Fineline 100 column, 15 cm bed height; 76 cm/hr flow rate) was done using the following protocol:

1. the column was equilibrated in 0.85 M ammonium sulphate, 50 mM sodium phosphate pH 7.0 buffer (buffer E).

2. the ammonium sulphate-diluted human acid a-glucosidase eluate from Phenyl HP was loaded on the column. The dynamic capacity was about 2 mg recombinant human acid a-glucosidase/ml Source 15 Phenyl.

3. after loading the sample, human acid a-glucosidase was eluted from the Source 15 Phenyl column with 10 cv of a linear gradient from 100% buffer E to 100% buffer F (buffer F 50 mM sodium phosphate buffer, pH 7.0). Careful pooling of the elution fraction is required (based on purity profiles of the column fractions on SDS-PAGE using Coomassie Brillant Blue staining) since some contaminating proteins elute directly after a-glucosidase.

4. residual bound proteins were eluted from the column with water and/or 20% ethanol.

A representative elution profile of a Source 15 Phenyl chromatography run is shown in FIG. 7. The recovery of human acid a-glucosidase activity in the pooled fraction (step 4) was generally >70%.

9. Final Filtration Steps: Ultra-, Dia-, Sterile-, and Nano-filtration

The pooled human acid a-glucosidase fractions from the Source 15 Phenyl column were concentrated by ultrafiltration in a TFF mode over a 0.1 m2 Biomax 30 membrane connected to the Proflux M12 system of Millipore.

After a 7-fold concentration, the retentate was diafiltered in 10 mM sodium phosphate buffer, pH 7 .0 (about 6 diafiltration volumes were used). Finally the acid a-glucosidase fraction was sterile filtered (0.2 um dead-end filters).

The recovery of human acid a-glucosidase after these filtration steps was >85%. Optionally a virus removal step can be incorporated: virus removal filters (nanofilters) like Planova 15 and 35 are feasible.

10. SDS-PAGE and HP-SEC Analysis of Purified Human Acid a-glucosidase

Purified human acid a-glucosidase was analyzed by silver-stained SDS/PAGE and Size-Exclusion HPLC (HP-SEC). FIG. 8 shows a Coomassie Brillant Blue-stained SDS-PAGE gel (4-12%, NuPage) of various milk fractions obtained during the purification run. Similar SDS-PAGE gels were visualized by silver-staining. A few minor bands were present. Western blotting of the gels with a polyclonal antibody against acid a-glucosidase, identified most of these minor bands as dimers and processed forms of the precursor acid a-glucosidase. At least 2 host-related impurities were present in the purified recombinant human acid a-glucosidase preparation.

The amount of these host-related impurities quantitated by densitometric scanning of the gel was around 1% of total protein loaded. Purified recombinant human acid a-glucosidase was also analyzed on a size exclusion column connected to a High Performance Liquid Chromatography System (HP-SEC). Results are shown in FIG. 9. The size exclusion column is able to separate proteins essentially on the basis of their molecular weight. Thus in principle this column is able to visualize and quantitate protein impurities of different molecular weights compared with the 110 kDa precursor a-glucosidase. As expected, the main protein peak was the recombinant human acid a-glucosidase precursor; peak surface analysis indicated that this peak was 99% of the total surface area of all visualized peaks. The molecular weight of the 110 kDa a-glucosidase monomer was estimated on this column to be 127 kDa. Some other small peaks were visible. On the basis of their elution profiles they were thought to have molecular weight of about 240 kDa (the 110 kDa a-glucosidase dimer), about 67 kDa (serum albumin), and about 20 kDa (unknown). Also some protein was present in the high molecular weight area.

11. Purification of Human Acid a-glucosidase Using Metal-chelating and Lectin Sepharoses

Various metal-chelating Sepharoses were prepared according to the recommendation of the manufacturer (Pharmacia). Radio-labeled human precursor acid a-glucosidase was incubated overnight with the various Sepharoses (for details: see example 1). After removal of the unbound label by washing, radioactivity bound to the beads was measured in a liquid scintillation counter. The results are shown in FIG. 10. Clearly, the Cu2+-chelating Sepharose is binding the radio-labeled human precursor acid a-glucosidase very good. Thus this ligand might be suitable for purification of the enzyme from milk and other sources, in contrast to the Fe2+, Fe3+ and Zn2+ Sepharoses.

The radio-labeled human precursor acid a-glucosidase also binds well to lectin Sepharoses like Concanavalin A (as expected), but unexpectedly also to lentil Sepharose (FIG. 9). Thus also lentil Sepharose is likely to be suitable for purification of acid a-glucosidase from milk.

12. Purification of Human Acid a-glucosidase Using Various HIC Media

Purified acid a-glucosidase and rabbit milk fractions were incubated with other HIC media than the Phenyl Sepharoses. In FIG. 11 the results are shown of chromatography experiments with column containing butyl, octyl, an ether ligands coupled to Sepharose (Pharmacia) and/or Toyopearl (TosoHaas) beads. Under conditions normal for HIC, a-glucosidase was found to bind more or less tightly to the various media.

13. Purification of Human Acid a-alucosidase Using Hydroxylapatite

Experiment 1

Hydroxylapatite was tried for its ability to separate recombinant human acid a-glucosidase from contaminating (whey) proteins. Hydroxylapatite is a crystalline form of calcium phosphate. Binding of proteins is mediated through the carboxyl and amino groups of the protein and Ca2+ and P04groups of the hydroxylapatite crystal lattice (Current protocols in Protein Science, eds. J. E. Coligan, B. M. Dunn, H. L. Ploegh, D. W. Speicher, P. T. Wingfield. John Wiley & Sons Inc. (1995), suppl.

Electrostatic interactions and specific effects are involved in the binding of neutral and acidic proteins to the Ca2+ sites, although the interaction of many proteins with hydroxylapatite can not be explained by the pl alone. DNA also binds to the matrix due to the charged phosphate backbone (Current protocols in Protein Science, eds. J. E. Coligan, B. M. Dunn, H. L. Ploegh, D. W. Speicher, P. T. Wingfield. John Wiley & Sons Inc. (1995), suppl. 8.6.9-8.6.12).

Transgenic and non-transgenic rabbit whey were loaded on a column containing ceramic hydroxylapatite type I (BioRad) at low salt concentration. After loading, bound protein was eluted with a gradient to 400 mM sodium phosphate (NaPj) pH 6.8. The chromatography profiles shown in FIG. 12 clearly show an increased flow through of the transgenic whey compared with the non-transgenic whey. SDS-PAGE analysis using silver staining (FIG. 13) clearly indicated that this fraction contains recombinant human acid a-glucosidase, together with WAP protein. Nearly all other whey proteins were bound to the column (the x axis of FIG. 12 shows the fraction numbers corresponding to the fraction numbers at the top of the lanes of the gels in FIG. 13). Acid a-glucosidase activity assays indicated that most activity was in the flow through fractions, and less than 5% was bound to the column.

These results clearly show that, unexpectedly, the heterologous protein (recombinant human) acid a-glucosidase does not bind to hydroxylapatite, while nearly all other whey proteins do. This means that the 1 ml (containing 2.5 mg protein) were individually loaded onto 2.5 mi ceramic Hydroxy Apatite (cHT) type 1 (BioRad) columns (bed height 15 cm). The column was washed after loading with 5 cv of an equilibration buffer in order to remove any unbound proteins. The bound proteins were then eluted with a sodium phosphate gradient from the molarity of the sample in question to 400 mM. 1.9 ml fractions were taken from the column eluate and then stored at 4 C. until SDS-PAGE analysis.

FIGS. 14 to 19 show the chromatographic traces obtained on hydroxylapatite chromatography of the whey samples 5, 10, 20, 30; 40 and 50 mM NaPi buffer, pH 7.0 respectively. In FIGS. 16, 17 and 18 (samples=30, 40 and 50mM NaPi, pH 7.0 respectively), the left hand peak on the trace represents at least a portion of the flow through material and the peak areas in these figures are significantly greater than those to be seen in from the corresponding peaks in FIGS. 14 and 19 (samples=5 and 60 mM NaPi, pH 7.0 respectively).

Silver stained SDS-PAGE analysis of fractions showed that at 5, 10 and 20 mM sodium phosphate the majority of the a-glucosidase was to be found in the flow through, whereas substantially all of the whey proteins were bound to the cHT beads. At 30, 40 and 50 mM NaPi the majority of the a-glucosidase remain in the flow through but the amount of whey protein in the flow through was increased.

The experiment shows how a good purification with acceptable recovery of protein can be achieved for a-glucosidase from transgenic whey samples at a sodium phosphate buffer sample concentration of between 5 and 20 mM. Where a greater purification with lesser recovery is required then a lower sample buffer concentration may be used.

15. Purification of Human Acid a-glucosidase Using Hydroxylapatite

Experiment 3

As noted in Experiment 2 above, transgenic rabbit whey containing about 3% (w/w) recombinant human acid a-glucosidase made by tangential flow filtration (TFF). The transgenic whey was diluted with water to give a final concentration of sodium phosphate (NaPj) buffer of 5mM at pH 7.2.500 II of the diluted whey containing about 2.5 mg protein was loaded on 2.5 mi ceramic Hydroxy Apatite (cHT) type 1 (BioRad) columns (bed height 15 cm).

Each column was equilibrated with 5 mM sodium phosphate buffer at pH 6.0, 6.5, 7.0 or 7.5. (FIGS. 20 to 23). After sample loading the column was washed with 5 cv of equilibration buffer. The bound proteins were eluted at a flow rate of 723 cm/hr with a gradient to 400 mM sodium phosphate buffer at pH 6.0, 6.5, 7.0 or 7.5 respectively. 1.0 ml fractions were analyzed for protein content by SDS-PAGE stained with silver.

Looking at the results of SDS-PAGE gels of the fractions stained with silver, one can see that at pH 6.0 a-glucosidase is only bound weakly to the cHT (ie there was some flow through), while substantially all whey proteins were bound more strongly to the cHT. At pH 6.5, about 90% of the a-glucosidase was in the flow through of the column and a low molecular weight (LMW) protein, probably whey acidic protein (WAP), was also in the flow through with the a-glucosidase but was somewhat retarded on the column. At pH 7.0, all of the a-glucosidase as well as most of the LMW protein (probably WAP) and the HMW proteins (probably Immunoglobulins) were in the flow through; an about 80 kD protein (probably transferrin) was also in the flow through but was somewhat retarded on the column.

Based on these results pH 6.5 would seem optimal for separation of a-glucosidase from whey proteins.

16. Purification of Human Acid a-glucosidase Using Q Sepharose FF and Hydroxylapatite

Transgenic whey (containing recombinant human acid a-glucosidase) made by tangential flow filtration was processed in a pilot plant facility by applying it to Q Sepharose FF (25 liter column volume) (Amersham Pharmacia Biotech) in 20 mM sodium phosphate (NaP;) pH 7.0 buffer. (FIG. 24). The column was equilibrated with 4 cv 50 mM NaPj, pH 7.0 and then 2 cv 20 mM NaPj, pH 7.0. The a-glucosidase containing fraction was eluted with 2.7 cv 0.1M NaCl pH 7.0. A 47.3 liter sample was taken and this contained 265 g protein. A sample of the 0.1M fraction was dialyse (3,500 Dalton molecular weight cut off, Spectra Por) against 10 mM sodium phosphate (NaPj) pH 6.5 buffer. 60 ml of the dialyse 0.1M sample (3.91 mg/ml protein, 1.33 mS/cm) was applied to a 30 ml cHT type 1 column (XK 16/15) (BioRad) at a flowrate of 150 cm/hr (5 ml/min). (FIG. 25). The column was washed after sample loading with 5 cv of equilibration buffer (10 mM NaPj, pH 6.5) 10 ml fractions were collected and the a-glucosidase was found in the flow through, whereas the majority of the whey proteins bound to the cHT beads. The bound proteins were eluted with a linear gradient of 10 to 400 mM sodium phosphate buffer. This step decreased the impurity levels in the aglucosidase containing Q Sepharose FF fraction from 90% to <0.5% in the flow through fraction of the cHT column. The recovery of a-glucosidase was greater than 80%.

FIG. 25 shows the chromatogram of the sample run on the cHT column.

FIG. 26 shows a silver stained SDS-PAGE gel showing the flow through fractions from cHT columns (lanes 1-3, 5-7 and 9-11); molecular weight standards (lane 4) and sample of QFF eluate loaded onto the cHT column (lane 12).

G. Uses of Recombinant Lysosomal Proteins

The recombinant lysosomal proteins produced according to the invention find use in enzyme replacement therapeutic procedures. A patient having a genetic or other deficiency resulting in an insufficiency of functional lysosomal enzyme can be treated by administering exogenous enzyme to the patient. Patients in need of such treatment can be identified from symptoms (e.g., Hurler's syndrome symptoms include Dwarfism, corneal clouding, hepatosplenomegaly, valvular lesions, coronary artery lesions, skeletal deformities, joint stiffness and progressive mental retardation). Alternatively, or additionally, patients can be diagnosed from biochemical analysis of a tissue sample to reveal excessive accumulation of a characteristic metabolite processed by a particular lysosomal enzyme or by enzyme assay using an artificial or natural substrate to reveal deficiency of a particular lysosomal enzyme activity. For most diseases, diagnosis can be made by measuring the particular enzyme deficiency or by DNA analysis before occurrence of symptoms or excessive accumulation of metabolites (Scriver et al., supra, chapters on lysosomal storage disorders). All of the lysosomal storage diseases are hereditary. Thus, in offspring from families known to have members suffering from lysosomal diseases, it is sometimes advisable to commence prophylactic treatment even before a definitive diagnosis can be made.

In some methods, lysosomal enzymes are administered in purified form together with a pharmaceutical carrier as a pharmaceutical composition. The preferred form depends on the intended mode of administration and therapeutic application. The pharmaceutical carrier can be any compatible, nontoxic substance suitable to deliver the polypeptides to the patient. Sterile water, alcohol, fats, waxes, and inert solids may be used as the carrier. Pharmaceutically-acceptable adjuvants, buffering agents, dispersing agents, and the like, may also be incorporated into the pharmaceutical compositions. The concentration of the enzyme in the pharmaceutical composition can vary widely, i.e., from less than about 0.1% by weight, usually being at least about 1% by weight to as much as 20% by weight or more.

For oral administration, the active ingredient can be administered in solid dosage forms, such as capsules, tablets, and powders, or in liquid dosage forms, such as elixirs, syrups, and suspensions. Active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate and the like. Examples of additional inactive ingredients that may be added to provide desirable color, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

A typical composition for intravenous infusion could be made up to contain 100 to 500 ml of sterile 0.9% NaCl or 5% glucose optionally supplemented with a 20% albumin solution and 100 to 500 mg of a enzyme. A typical pharmaceutical compositions for intramuscular injection would be made up to contain, for example, 1 ml of sterile buffered water and 1 to 10 mg of the purified ligand of the present invention. Methods for preparing parenterally administrable compositions are well known in the art and described in more detail in various sources, including, for example, Remington's Pharmaceutical Science (15^{th} ed., Mack Publishing, Easton, Pa., 1980) (incorporated by reference in its entirety for all purposes).

AGLU can be formulated in 10 mM sodium phosphate buffer pH 7.0. Small amounts of ammonium sulphate are optionally present (<10 mM). The enzyme is typically kept frozen at about −70 C., and thawed before use. Alternatively, the enzyme may be stored cold (e.g., at about 4 C. to 8 C.) in solution. In some embodiments, AGLU solutions comprise a buffer (e.g., sodium phosphate, potassium phosphate or other physiologically acceptable buffers), a simple carbohydrate (e.g., sucrose, glucose, maltose, mannitol or the like), proteins (e.g., human serum albumin), and/or surfactants (e.g., polysorbate 80 (Tween-80), cremophore-EL, cremophore-R, labrofil, and the like).

AGLU can also be stored in lyophilized form. For lyophilization, AGLU can be formulated in a solution containing mannitol, and sucrose in a phosphate buffer.

The concentration of sucrose should be sufficient to prevent aggregation of AGLU on reconstitution. The concentration of mannitol should be sufficient to significantly reduce the time otherwise needed for lyophilization. The concentrations of mannitol and sucrose should, however, be insufficient to cause unacceptable hypertonicity on reconstitution.

Concentrations of mannitol and sucrose of 1-3 mg/ml and 0.1-1.0 mg/ml respectively are suitable. Preferred concentrations are 2 mg/ml mannitol and 0.5 mg/ml sucrose. AGLU is preferably at 5 mg/ml before lyophilization and after reconstitution. Saline preferably at 0.9% is a preferred solution for reconstitution.

For AGLU purified from rabbit milk, a small amount of impurities (e.g., up to about 5%) can be tolerated. Possible impurities may be present in the form of rabbit whey proteins. Other possible impurities are structural analogues (e.g., oligomers and aggregates) and truncations of AGLU. Current batches indicate that the AGLU produced in transgenic rabbits is >95% pure. The largest impurities are rabbit whey proteins, although on gel electrophoresis, AGLU bands of differing molecular weights are also seen.

Infusion solutions should be prepared aseptically in a laminar air flow hood.

The appropriate amount of AGLU should be removed from the freezer and thawed at room temperature. Infusion solutions can be prepared in glass infusion bottles by mixing the appropriate amount of AGLU finished product solution with an adequate amount of a solution containing human serum albumin (HSA) and glucose. The final concentrations can be 1% HSA and 4% glucose for 25-200 mg doses and 1% HSA and 4% glucose for 400-800 mg doses. HSA and AGLU can be filtered with a 0.2 pm syringe filter before transfer into the infusion bottle containing 5% glucose. Alternatively, AGLU can be reconstituted in saline solution, preferably 0.9% for infusion. Solutions of AGLU for infusion have been shown to be stable for up to 7 hours at room temperature. Therefore the AGLU solution is preferably infused within seven hours of preparation.

The pharmaceutical compositions of the present invention are usually administered intravenously. Intradermal, intramuscular or oral administration is also possible in some circumstances. The compositions can be administered for prophylactic treatment of individuals suffering from, or at risk of, a lysosomal enzyme deficiency disease. For therapeutic applications, the pharmaceutical compositions are administered to a patient suffering from established disease in an amount sufficient to reduce the concentration of accumulated metabolite and/or prevent or arrest further accumulation of metabolite. For individuals at risk of lysosomal enzyme deficiency disease, the pharmaceutical composition are administered prophylactically in an amount sufficient to either prevent or inhibit accumulation of metabolite. An amount adequate to accomplish this is defined as a “therapeutically-” or “prophylactically-effective dose.” Such effective dosages will depend on the severity of the condition and on the general state of the patient's health, but will generally range from about 0.1 to 10 mg of purified enzyme per kilogram of body weight.

In the present methods, human acid alpha glucosidase is usually administered at a dosage of 10 mg/kg patient body weight or more per week to a patient. Often dosages are greater than 10 mg/kg per week. Dosages regimes can range from 10 mg/kg per week to at least 1000 mg/kg per week. Typically dosage regimes are 10 mg/kg per week, 15 mg/kg per week, 20 mg/kg per week, 25 mg/kg per week, 30 mg/kg per week, 35 mg/kg per week, 40 mg/kg week, 45 mg/kg per week, 60 mg/kg week, 80 mg/kg per week and 120 mg/kg per week. In preferred regimes 10 mg/kg, 15 mg/kg, 20 mg/kg, 30 mg/kg or 40 mg/kg is administered once, twice or three times weekly. Treatment is typically continued for at least 4 weeks, sometimes 24 weeks, and sometimes for the life of the patient. Treatment is preferably administered i.v. Optionally, levels of human alpha-glucosidase are monitored following treatment (e.g., in the plasma or muscle) and a further dosage is administered when detected levels fall substantially below (e.g., less than 20%) of values in normal persons.

In some methods, human acid alpha glucosidase is administered at an initially “high” dose (i.e., a “loading dose”), followed by administration of a lower doses (i.e., a “maintenance dose”). An example of a loading dose is at least about 40 mg/kg patient body weight 1 to 3 times per week (e.g., for 1, 2, or 3 weeks). An example of a maintenance dose is at least about 5 to at least about 10 mg/kg patient body weight per week, or more, such as 20 mg/kg per week, 30 mg/kg per week, 40 mg/kg week.

In some methods, a dosage is administered at increasing rate during the dosage period. Such can be achieved by increasing the rate of flow intravenous infusion or by using a gradient of increasing concentration of alpha-glucosidase administered at constant rate. Administration in this manner reduces the risk of immunogenic reaction. In some dosages, the rate of administration measured in units of alpha glucosidase per unit time increases by at least a factor of ten. Typically, the intravenous infusion occurs over a period of several hours (e.g., 1-10 hours and preferably 2-8 hours, more preferably 3-6 hours), and the rate of infusion is increased at intervals during the period of administration.

Suitable dosages (all in mg/kg/hr) for infusion at increasing rates are shown in table 1 below. The first column of the table indicates periods of time in the dosing schedule.

For example, the reference to 0-1 hr refers to the first hour of the dosing. The fifth column of the table shows the range of doses than can be used at each time period. The fourth column shows a narrower included range of preferred dosages. The third column indicates upper and lower values of dosages administered in an exemplary clinical trial. The second column shows particularly preferred dosages, these representing the mean of the range shown in the third column of table 1.

TABLE 1

| | | Lower&Upper | Preferred | |
|---|---|---|---|---|
| Time | Mean Doses (I) | Value | Range | Range |
| 0-1 hr: | 0.3 mg/kg/hr | 0.25-0.4 | 0.1-1 | 0.03-3 |
| 1-2 hr: | 1 mg/kg/hr | 0.9-1.4 | 1-4 | 0.3-12 |
| 2-2.5 hr: | 4 mg/kg/hr | 3.6-5.7 | 3-10 | 1-30 |
| 2.5-5.8 hr: | 12 mg/kg/hr | 7.2-11.3 | 6-20 | 2-50 |

The methods are effective on patients with both early onset (infantile) and late onset (juvenile and adult) Pompe's disease. In patients with the infantile form of Pompe's disease symptoms become apparent within the first 4 months of life. Mostly, poor motor development and failure to thrive are noticed first. On clinical examination, there is generalized hypotonia with muscle wasting, increased respiration rate with sternal retractions, moderate enlargement of the liver, and protrusion of the tongue. Ultrasound examination of the heart shows a progressive hypertrophic cardiomyopathy, eventually leading to insufficient cardiac output. The ECG is characterized by marked left axis deviation, a short PR interval, large QRS complexes, inverted T waves and ST depression. The disease shows a rapidly progressive course leading to cardiorespiratory failure within the first year of life. On histological examination at autopsy lysosomal glycogen storage is observed in various tissues, and is most pronounced in heart and skeletal muscle. Treatment with human acid alpha glucosidase in the present methods results in a prolongation of life of such patients (e.g., greater than 1, 2, 5 years up to a normal lifespan). Treatment can also result in elimination or reduction of clinical and biochemical characteristics of Pompe's disease as discussed above. Treatment is administered soon after birth, or antenatally if the parents are known to bear variant alpha glucosidase alleles placing their progeny at risk.

Patients with the late onset adult form of Pompe's disease may not experience symptoms within the first two decades of life. In this clinical subtype, predominantly skeletal muscles are involved with predilection of those of the limb girdle, the trunk and the diaphragm. Difficulty in climbing stairs is often the initial complaint. The respiratory impairment varies considerably. It can dominate the clinical picture, or it is not experienced by the patient until late in life. Most such patients die because of respiratory insufficiency. In patients with the juvenile subtype, symptoms usually become apparent in the first decade of life. As in adult Pompe's disease, skeletal muscle weakness is the major problem; cardiomegaly, hepatomegaly, and macroglossia can be seen, but are rare. In many cases, nightly ventilatory support is ultimately needed. Pulmonary infections in combination with wasting of the respiratory muscles are life threatening and mostly become fatal before the third decade. Treatment with the present methods prolongs the life of patients with late onset juvenile or adult Pompe's disease up to a normal life span. Treatment also eliminates or significantly reduces clinical and biochemical symptoms of disease.

Lysosomal proteins produced in the milk of transgenic animals have a number of other uses. For example, .alpha.-glucosidase, in common with other .alpha.-amylases, is an important tool in production of starch, beer and pharmaceuticals. See Vihinen & Mantsala, Crit. Rev. Biochem. Mol. Biol. 24, 329-401 (1989) (incorporated by reference in its entirety for all purpose). Lysosomal proteins are also useful for producing laboratory chemicals or food products. For example, acid .alpha.-glucosidase degrades 1,4 and 1,6 .alpha.-glucosidic bounds and can be used for the degradation of various carbohydrates containing these bonds, such as maltose, isomaltose, starch and glycogen, to yield glucose. Acid .alpha.-glucosidase is also useful for administration to patients with an intestinal maltase or isomaltase deficiency. Symptoms otherwise resulting from the presence of undigested maltose are avoided. In such applications, the enzyme can be administered without prior fractionation from milk, as a food product derived from such milk (e.g., ice cream or cheese) or as a pharmaceutical composition. Purified recombinant lysosomal enzymes are also useful for inclusion as controls in diagnostic kits for assay of unknown quantities of such enzymes in tissue samples.

Therapeutic Methods

The present invention provides effective methods of treating Pompe's disease.

These methods are premised in part on the availability of large amounts of human acid alpha glucosidase in a form that is catalytically active and in a form that can be taken up by tissues, particularly, liver, heart and muscle (e.g., smooth muscle, striated muscle, and cardiac muscle), of a patient being treated. Such human acid alpha-glucosidase is provided from e.g., the transgenic animals described in the Examples. The alpha-glucosidase is preferably predominantly (i.e., >50%) in the precursor form of about 100-10 kD. (The apparent molecular weight or relative mobility of the 100-110 kD precursor may vary somewhat depending on the method of analysis used, but istypically within the range 95 kD and 120 kD.) Given the successful results with human acid alpha-glucosidase in the transgenic animals discussed in the Examples, it is possible that other sources of human alphaglucosidase, such as resulting from cellular expression systems, can also be used. For example, an alternative way to produce human acid a-glucosidase is to transfect the acid aglucosidase gene into a stable eukaryotic cell line (e.g., CHO) as a cDNA or genomic construct operably linked to a suitable promoter. However, it is more laborious to produce the large amounts of human acid alpha glucosidase needed for clinical therapy by such an approach.

### EXAMPLES

### Example 1

### Construction of Transgenes

(a) cDNA Construct

Construction of an expression vector containing cDNA encoding human acid .alpha.-glucosidase started with the plasmid p16,8hlf3 (see DeBoer et al. (1991) & (1993), supra) This plasmid includes bovine .alpha.S1-casein regulatory sequences. The lactoferrin cDNA insert of the parent plasmid was exchanged for the human acid .alpha.-glucosidase cDNA (Hoefsloot et al. EMBO J. 7,1697-1704 (1988)) at the ClaI site and SalI site of the expression cassette as shown in FIG. 1. To obtain the compatible restriction sites at the ends of the .alpha.-glucosidase cDNA fragment, plasmid pSHAG2 (id.) containing the complete cDNA encoding human .alpha.-glucosidase was digested with EcoRI and SphI and the 3.3 kb cDNA-fragment was subcloned in pKUN7.DELTA.C a pKUN1 derivative (Konings et al., Gene 46, 269-276 (1986)), with a destroyed ClaI site within the vector nucleotide sequences and with a newly designed polylinker: HindIII ClaI EcoRI SphI XhoI EcoRI SfiI SfiI/SmaI NotI EcoRI*(*=destroyed site). From the resulting plasmid p.alpha.gluCESX, the 3.3-kb cDNA-fragment could be excised by ClaI and XhoI. This fragment was inserted into the expression cassette shown in FIG. 1 at the ClaI site and XhoI-compatible SalI site. As a result, the expression plasmid p16,8.alpha.glu consists of the cDNA sequence encoding human acid .alpha.-glucosidase flanked by bovine alpha.S1-casein sequences as shown in FIG. 1. The 27.3-kb fragment containing the complete expression cassette can be excised by cleavage with NotI (see FIG. 1).

(b) Genomic Constructs

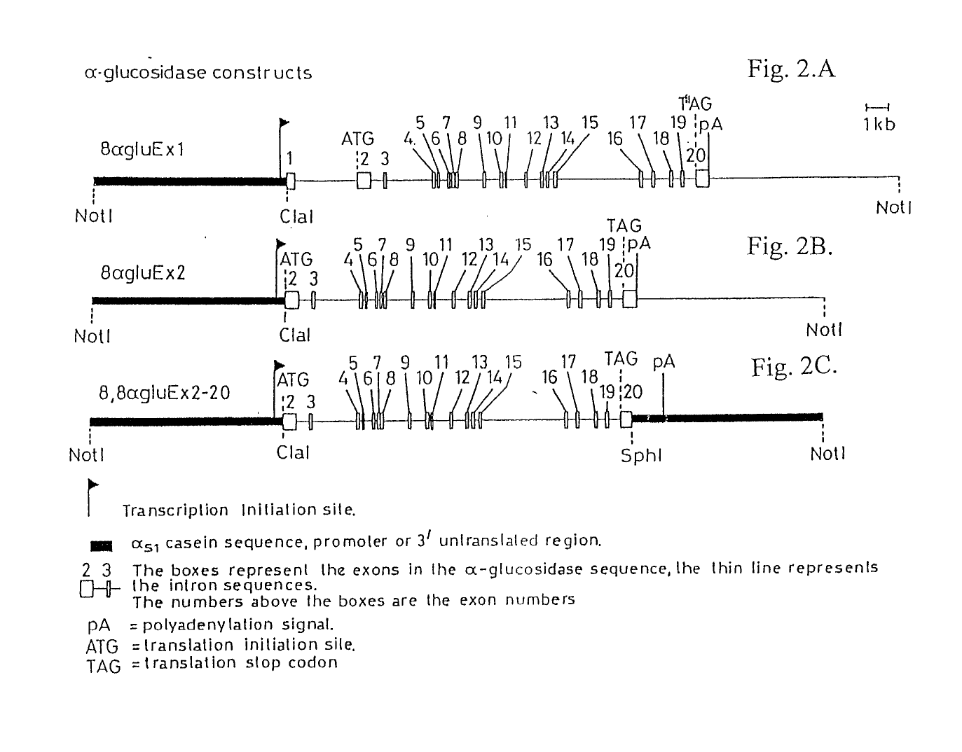

Construct c8.alpha.gluex1 contains the human acid .alpha.-glucosidase gene (Hoefsloot et al., Biochem. J. 272, 493--497 (1990)); starting in exon 1 just downstream of its transcription initiation site (see FIG. 2, panel A). Therefore, the construct encodes almost a complete 5′ UTR of the human acid .alpha.-glucosidase gene. This fragment was fused to the promoter sequences of the bovine .alpha.S1-casein gene. The .alpha.S1-casein initiation site is present 22 bp upstream of the .alpha.S1-casein/acid .alpha.-glucosidase junction. The construct has the human acid .alpha.-glucosidase polyadenylation signal and 3′ flanking sequences. Construct c8.alpha.gluex2 contains the bovine .alpha.S1-casein promoter immediately fused to the translation initiation site in exon 2 of the human acid .alpha.-glucosidase gene (see FIG. 2, panel B). Thus, the .alpha.S1-casein transcription initiation site and the .alpha.-glucosidase translation initiation site are 22-bp apart in this construct. Therefore no .alpha.-glucosidase 5′ UTR is preserved. This construct also contains the human acid .alpha.-glucosidase polyadenylation signal and 3′ flanking sequences as shown in FIG. 2, panel B.

Construct c8,8.alpha.gluex2-20 differs from construct c8.alpha.gluex2 only in the 3′ region. A SphI site in exon 20 was used to fuse the bovine .alpha.S1-casein 3′ sequence to the human acid .alpha.-glucosidase construct. The polyadenylation signal is located in this 3′ .alpha.S1-casein sequence (FIG. 2, panel C).

Methods for Construction of Genomic Constructs

Three contiguous BglII fragments containing the human acid .alpha.-glucosidase gene were isolated by Hoefsloot et al., supra. These fragments were ligated in the BglII-site of pKUN8.DELTA.C, a pKUN7.DELTA.C derivative with a customized polylinker: HindIII ClaI StuI SstI BglII PvnI NcoI EcoRI SphI XhoI EcoRI* SmaI/SfiI NotI EcoRI* (*=destroyed site). This ligation resulted in two orientations of the fragments generating plasmids p7.3.alpha.gluBES, p7.3.alpha.gluBSE, p8.5.alpha.gluBSE, p8.5.alpha.gluBES, p10.alpha.agluBSE and p10.alpha.gluBES.

Because unique NotI-sites at the ends of the expression cassette are used subsequently for the isolation of the fragments used for microinjection, the intragenic NotI site in intron 17 of human acid .alpha.-glucosidase had to be destroyed. Therefore, p10.alpha.gluBES was digested with ClaI and XhoI; the fragment containing the 3′ .alpha.-glucosidase end was isolated. This fragment was inserted in the ClaI and XhoI sites of pKUN10.DELTA.C, resulting in p10.alpha.glu.DELTA.NV. Previously pKUN10.DELTA.C (i.e., a derivative of pKUN8.DELTA.C) was obtained by digesting pKUN8.DELTA.C with NotI; filling in the sticky ends with Klenow and subsequently, annealing the plasmid by blunt-ended ligation. Finally, p10.alpha.glu.DELTA.NV was digested with NotI. These sticky ends were also filled with Klenow and the fragment was ligated, resulting in plasmid p10.alpha.glu.DELTA.NotI.

Construction of C8.Alpha.Gluex1

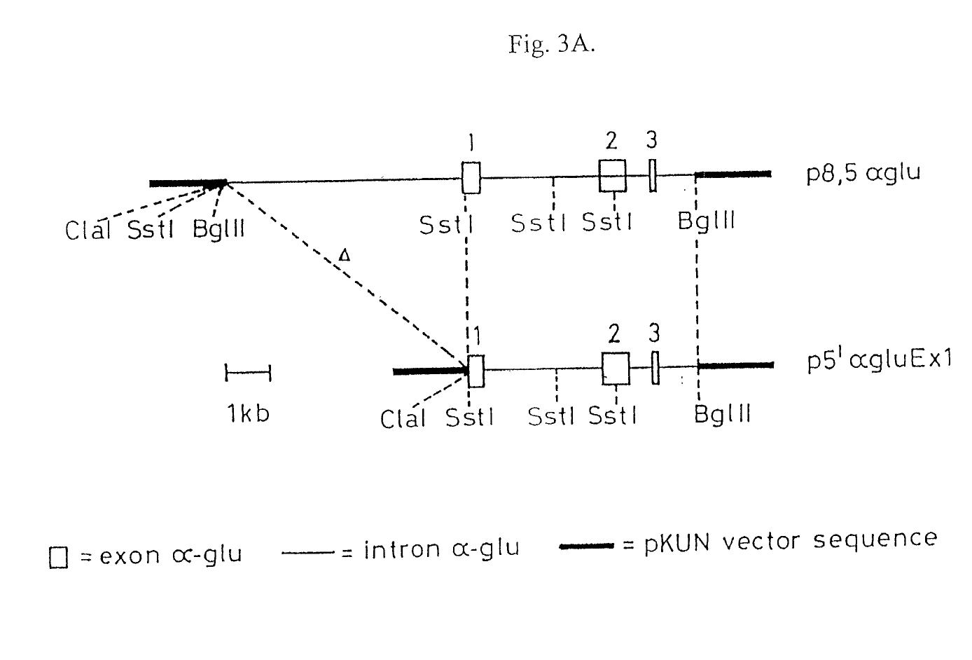

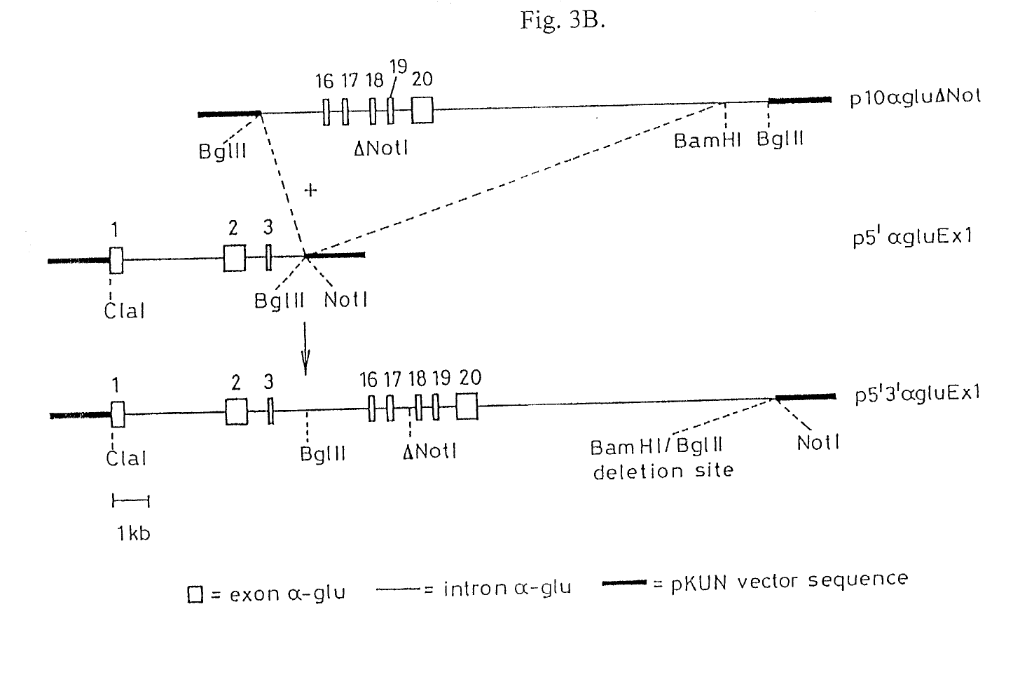

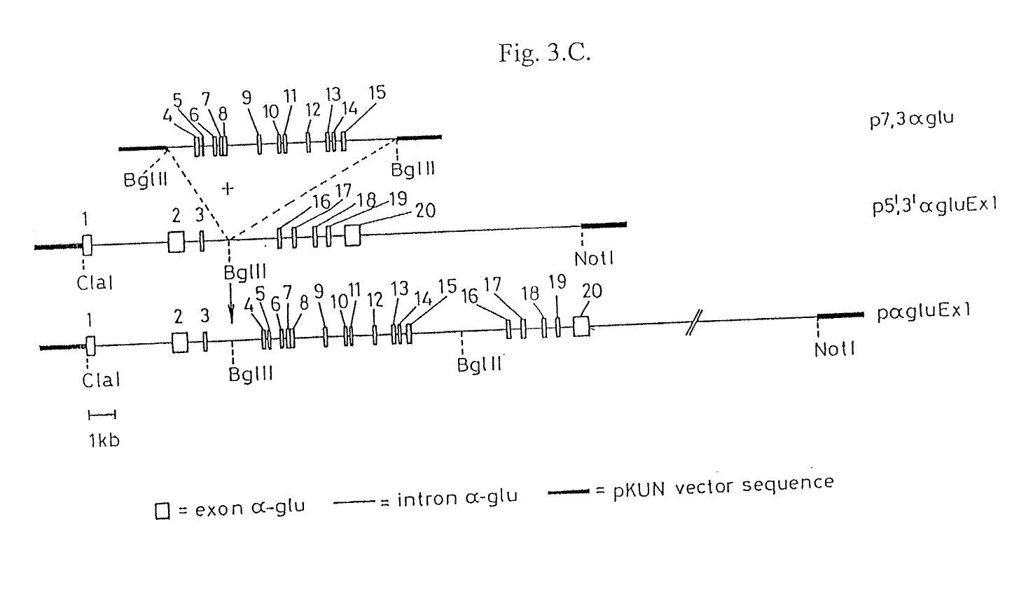

Since the SstI site in first exon of the .alpha.-glucosidase gene was chosen for the fusion to the bovine .alpha.S1-casein sequence, p8.5.alpha.gluBSE was digested with BglII followed by a partial digestion with SstI. The fragment containing exon 1-3 was isolated and ligated into the BglII and SstI sites of pKUN8.DELTA.C. The resulting plasmid was named: p5′.alpha.gluex1 (see FIG. 3, panel A).

The next step (FIG. 3, panel B) was the ligation of the 3 ′ part to p5′.alpha.gluex1. First, p10.alpha.gluAN was digested with BglII and BamHI. This fragment containing exon 16-20 was isolated. Second, p5′.alpha.gluex1 was digested with BglII and to prevent self-ligation, and treated with phosphorylase (BAP) to dephosphorylate the sticky BglII ends. Since BamHI sticky ends are compatible with the BglII sticky ends, these ends ligate to each other. The resulting plasmid, i.e., p5′3′.alpha.gluex1, was selected. This plasmid has a unique BglII site available for the final construction step (see FIG. 3, panels B and C). The middle part of the .alpha.-glucosidase gene was inserted into the latter construct. For this step, p7.3.alpha.gluBSE was digested with BglII, the 8.5-kb fragment was isolated and ligated to the BglII digested and dephosphorylated p5′3′.alpha.gluex1 plasmid. The resulting plasmid is p.alpha.gluex1 (FIG. 3, panel C).

The bovine .alpha.S1-casein promoter sequences were incorporated in the next step via a ligation involving three fragments. The pWE15 cosmid vector was digested with NotI and dephosphorylated. The bovine .alpha.s1-casein promoter was isolated as an 8 Rb NotI-ClaI fragment (see de Boer et al., 1991, supra). The human acid .alpha.-glucosidase fragment was isolated from p.alpha.gluex1using the same enzymes. These three fragments were ligated and packaged using the Stratagene GigapackII kit in 1046 E.coli host cells. The resulting cosmid c8.alpha.gluex1 was propagated in E.coli strain DH5.alpha. The vector was linearized with NotI before microinjection.

Construction of C8.Alpha.Gluex2 and C8,8.Alpha.Gluex2-20

The construction of the other two expression plasmids (FIG. 2, panels B and C) followed a similar strategy to that of c8.alpha.gluex1. The plasmid p5′.alpha.gluStuI was derived from p8,5.alpha.gluBSE by digestion of the plasmid with StuI, followed by self-ligation of the isolated fragment containing exon 2-3 plus the vector sequences. Plasmid p5′.alpha.gluStuI was digested with PglII followed by a partial digestion of the linear fragment with NcoI resulting in several fragments. The 2.4 kb fragment, containing exon 2 and 3, was isolated and ligated into the NcoI and BglII sites of vector pKUN12.DELTA.C, resulting in p5′.alpha.gluex2. Note that pKUN12.DELTA.C is a derivative of pKUN8.DELTA.C containing the polylinker: ClaI NcoI BglII HindIII EcoRI SphI XhoI SmaI/SfiI NotI.

The plasmid p10.alpha.glu.DELTA.NotI was digested with BglII and HindIII. The fragment containing exons 16-20 was isolated and ligated in p5′.alpha.gluex2 digested with BglIII and HindIII. The resulting plasmid was p5′3 ′.alpha.gluex2. The middle fragment in p5′3′.alpha.gluex2 was inserted as for p.alpha.gluex1. For this, p7.3.alpha.glu was digested with BglII. The fragment was isolated and ligated to the BglII-digested and dephosphorylated p5′3.alpha.gluex2. The resulting plasmid, p.alpha.gluex2, was used in construction of c8.alpha.gluex-20 and c8,8.alpha.gluex2-20 (FIG. 2, panels B and C).

For the construction of third expression plasmid c8,8.alpha.gluex2-20 (FIG. 2, panel C) the 3′ flanking region of .alpha.-glucosidase was deleted. To achieve this, p.alpha.gluex2 was digested with SphI. The fragment containing exon 2-20 was isolated and self-ligated resulting in p.alpha.gluex2-20. Subsequently, the fragment containing the 3′ flanking region of bovine .alpha.s1-casein gene was isolated from p16,8.alpha.glu by digestion with SphI and NotI. This fragment was inserted into p.alpha.gluex2-20 using the SphI site and the NotI site in the polylinker sequence resulting in p.alpha.gluex2-20-3.alpha.S1.

The final step in generating c8,8.alpha.gluex2-20 was the ligation of three fragments as in the final step in the construction leading to c8.alpha.gluex1. Since the ClaI site in p.alpha.gluex2-20-3′.alpha.S1 and p.alpha.gluex2 appeared to be uncleavable due to methylation, the plasmids had to be propagated in the E. coli DAM(−) strain ECO343. The p.alpha.gluex2-20-3 ′.alpha.S 1 isolated from that strain was digested with ClaI and NotI. The fragment containing exons 2-20 plus the 3′.alpha.S 1-casein flanking region was purified from the vector sequences. This fragment, an 8 kb NotI-ClaI fragment containing the bovine .alpha.s1 promoter (see DeBoer (1991) & (1993), supra) and NotI-digested, dephosphorylated pWE15 were ligated and packaged. The resulting cosmid is c8,8.alpha.gluex2-20.

Cosmid c8.alpha.gluex2 (FIG. 2, panel B) was constructed via a couple of different steps. First, cosmid c8,8.alpha.gluex2-20 was digested with SalI and NotI. The 10.5-kb fragment containing the .alpha.S1-promoter and the exons 2-6 part of the acid .alpha.-glucosidase gene was isolated. Second, plasmid p.alpha.gluex2 was digested with SalI and NotI to obtain the fragment containing the 3 ′ part of the acid .alpha.-glucosidase gene. Finally, the cosmid vector pWE15 was digested with NotI and dephosphorylated. These three fragments were ligated and packaged. The resulting cosmid is c8.alpha.gluex2.

### Example 2

### Transgenesis

The cNA and genomic constructs were linearized with NotI and injected in the pronucleus of fertilized mouse oocytes which were then implanted in the uterus of pseudopregnant mouse foster mothers. The offspring were analyzed for the insertion of the human .alpha.-glucosidase cDNA or genomic DNA gene construct by Southern blotting of DNA isolated from clipped tails. Transgenic mice were selected and bred.

The genomic constructs linearized with NotI were also injected into the pronucleus of fertilized rabbit oocytes, which were implanted in the uterus of pseudopregnant rabbit foster mothers. The offspring were analyzed for the insertion of the alpha-glucosidase DNA by Southern blotting. Transgenic rabbits were selected and bred.

### Example 3

### Analysis of Acid .alpha.-Glucosidase in the Milk of Transgenic Mice

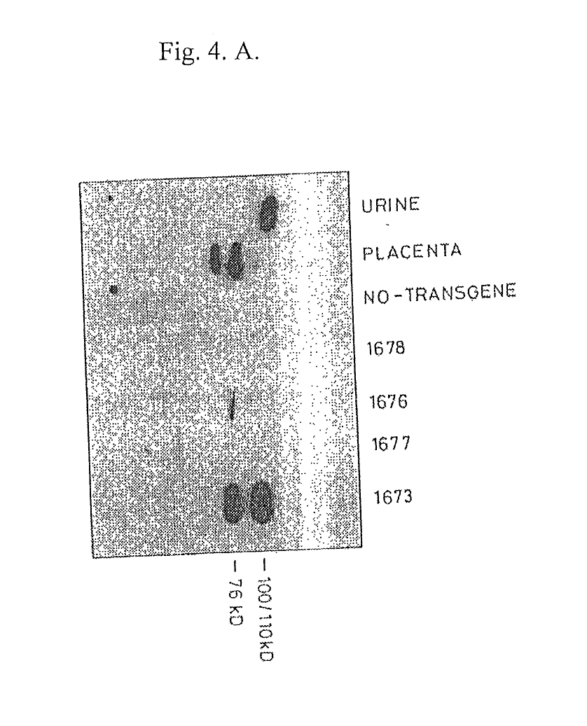

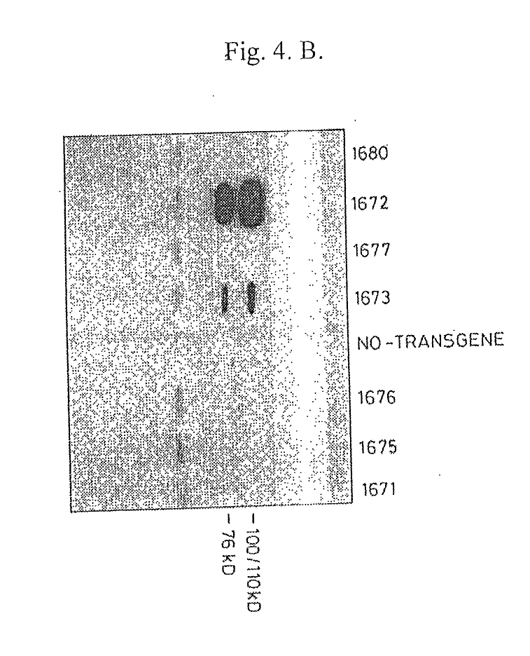

Milk from transgenic mice and nontransgenic controls was analyzed by Western Blotting. The probe was mouse antibody specific for human acid .alpha.-glucosidase (i.e., does not bind to the mouse enzyme). Transgenes 1672 and 1673 showed expression of human acid .alpha.-glucosidase in milk (FIG. 4). Major and minor bands at 100-110 kD and 76 kD were observed as expected for .alpha.-glucosidase. In milk from non-transgenic mice, no bands were observed.

The activity of human acid .alpha.-glucosidase was measured with the artificial substrate 4-methylumbelliferyl-.alpha.-D-glucopyranoside in the milk of transgenic mouse lines (See Galiaard, Genetic Metabolic Disease, Early Diagnosis and Prenatal Analysis, Elsevier/North Holland, Amsterdam, pp. 809-827 (1980)). Mice containing the cDNA construct (FIG. 1) varied from 0.2 to 2 .mu.mol/ml per hr. The mouse lines containing the genomic construct (FIG. 2, panel A) expressed at levels from 10 to 610 .mu.mol/ml per hr. These figures are equivalent to a production of 1.3 to 11.3 mg/l (cDNA construct) and 0.05 to 3.3 g/l (genomic construct) based on an estimated specific activity of the recombinant enzyme of 180 .mu.mol/mg (Van der Ploeg et al., J. Neurol. 235:392-396 (1988)).

The recombinant acid .alpha.-glucosidase was isolated from the milk of transgenic mice, by sequential chromatography of milk on ConA-Sepharose.™. and Sephadex.™. G200. 7 ml milk was diluted to 10 ml with 3 ml Con A buffer consisting of 10 mM sodium phosphate, pH 6.6 and 100 mM NaCl. A 1:1 suspension of Con A sepharose in Con A buffer was then added and the milk was left overnight at 4.degree. C. with gentle shaking. The Con A sepharose beads were then collected by centrifugation and washed 5 times with Con A buffer, 3 times with Con A buffer containing 1 M NaCl instead of 100 mM, once with Con A buffer containing 0.5 M NaCl instead of 100 mM and then eluted batchwise with Con A buffer containing 0.5 M NaCl and 0.1 M methyl-.alpha.-D-mannopyranoside. The acid .alpha.-glucosidase activity in the eluted samples was measured using the artificial 4-methylumbelliferyl-.alpha.-D-glycopyranoside substrate (see above). Fractions containing acid .alpha.-glucosidase activity were pooled, concentrated and dialyzed against Sephadex buffer consisting of 20 mM Na acetate, pH 4.5 and 25 mM NaCl, and applied to a Sephadex.™. 200 column. This column was run with the same buffer, and fractions were assayed for acid .alpha.-glucosidase activity and protein content. Fractions rich in acid .alpha.-glucosidase activity and practically free of other proteins were pooled and concentrated. The method as described is essentially the same as the one published by Reuser et al., Exp. Cell Res. 155:178-179 (1984). Several modifications of the method are possible regarding the exact composition and pH of the buffer systems and the choice of purification steps in number and in column material.

Acid alpha.-glucosidase purified from the milk was then tested for phosphorylation by administrating the enzyme to cultured fibroblasts from patients with CSD II (deficient in endogenous acid .alpha.-glucosidase). In this test mannose 6-phosphate containing proteins are bound by mannose 6-phosphate receptors on the cell surface of fibroblasts and are subsequently internalized. The binding is inhibited by free mannose 6-phosphate (Reuser et al., Exp. Cell Res. 155:178-189 (1984)). In a typical test for the phosphorylation of acid .alpha.-glucosidase isolated from milk of transgenic mice, the acid .alpha.-glucosidase was added to 10.sup.4-10.sup.6 fibroblasts in 500 .mu.l culture medium (Ham F10, supplied with 10% fetal calf serum and 3 mM Pipes) in an amount sufficient to metabolize 1 .mu.mole 4-methyl-umbelliferyl-.alpha.-D-glucopyranoside per hour for a time period of 20 hours. The experiment was performed with or without 5 mM mannose 6-phosphate as a competitor, essentially as described by Reuser et al., supra (1984). Under these conditions acid .alpha.-glucosidase of the patient fibroblasts was restored to the level measured in fibroblasts from healthy individuals. The restoration of the endogenous acid .alpha.-glucosidase activity by acid .alpha.-glucosidase isolated from mouse milk was as efficient as restoration by acid .alpha.-glucosidase purified from bovine testis, human urine and medium of transfected CHO cells. Restoration by .alpha.-glucosidase form milk was inhibited by 5 mM mannose 6-phosphate as observed for .alpha.-glucosidase from other sources. (Reuser et al., supra; Van der Ploeg et al., (1988), supra; Van der Ploeg et al., Ped. Res. 24:90-94 (1988).

As a further demonstration of the authenticity of .alpha.-glucosidase produced in the milk, the N-terminal amino acid sequence of the recombinant .alpha.-glucosidase produced in the milk of mice was shown to be the same as that of .alpha.-glucosidase precursor from human urine as published by Hoefsloot et al., EMBO J. 7:1697-1704 (1988) which starts with AHPGRP.

### Example 4

### Animal Trial of Alpha-Glucosidase

Recently, a knock-out mouse model for Pompe's disease has become available (25) This model was generated by targeted disruption of the murine alpha-glucosidase gene.

Glycogen-containing lysosomes are detected soon after birth in liver, heart and skeletal muscle. Overt clinical symptoms only become apparent at relatively late age (>9 months), but the heart is typically enlarged and the electrocardiogram is abnormal.

Experiments have been carried out using the knock-out (KO) mouse model in order to study the in vivo effect of AGLU purified from transgenic rabbit milk. The recombinant enzyme in these experiments was purified from milk of the transgenic rabbits essentially as described above for purification from transgenic mice.

1. Short Term Studies in KO Mouse Model

Single or multiple injections with a 6 day interval were administered to KO mice via the tail vein. Two days after the last enzyme administration the animals were killed, and the organs were perfused with phosphate buffered saline (PBS). Tissue homogenates were made for GLU enzyme activity assays and tissue glycogen content, and ultrathin sections of various organs were made to visualize accumulation (via electron microscopy) lysosomal glycogen content. Also the localization of internalized AGLU was determined using rabbit polyclonal antibodies against human placenta mature a-glucosidase.

The results showed that single doses of 0.7 and 1.7 mg AGLU (experiments C and A respectively) was taken up efficiently in vivo in various organs of groups of two knock-out mice when injected intravenously. Experiment A also showed that there were no differences in the uptake and distribution of AGLU purified from two independent rabbit milk sources.

Increases in AGLU activity were seen in the organs such as the liver, spleen, heart, and skeletal muscle, but not in the brain. Two days after a single injection of 1.7 mg AGLU to two KO animals, levels close to, or much higher than, the endogenous alphaglucosidase activity levels observed in organs of two PBS-injected normal control mice or two heterozygous KO mice were obtained (experiment A). Of the organs tested, the liver and spleen are, quantitatively, the main organs involved in uptake, but also the heart and pectoral and femoral muscles take up significant amounts of enzyme. The absence of a significant increase in brain tissue suggests that AGLU does not pass the blood-brain barrier. The results are summarized in Table 2.

TABLE 2
Tissue Uptake of AGLU and Glycogen Content Following Short Term Treatment in KO Mouse Model

| | | | | | | | Pectoral | | Femoral | | | | | |
|---|---|---|---|---|---|---|---|---|---|---|---|---|---|---|
| | Liver | | Spleen | | Heart | | Muscle | | Muscle | | Tongue | | Brain | |
| Group | Act | Glc | Act | Glc | Act | Glc | Act | Glc | Act | Glc | Act | Glc | Act | Glc |

Experiment A animals treated with single dose of 1.7 mg AGLU (from 2 sources)

| | | | | | | | | | | | | | | |
|---|---|---|---|---|---|---|---|---|---|---|---|---|---|---|
| treated KO | 674 | — | — | — | 263 | — | — | — | 24 | — | — | — | 0.8 | — |
| mice source 1 | 410 | | | | 17 | | | | 3.1 | | | | 0.4 | |
| treated KO | 454 | — | — | — | 76 | — | — | — | 12 | — | — | — | 0.8 | — |
| mice source 2 | 604 | | | | 48 | | | | 10 | | | | 0.4 | |
| untreated KO | 3.1 | — | — | — | 0.2 | — | — | — | 0.2 | — | — | — | 0.2 | — |
| mouse | | | | | | | | | | | | | | |
| untreated | 58 | — | — | — | 23 | — | — | — | 11 | — | — | — | 57 | — |
| normal mouse | 37 | | | | 17 | | | | 8.2 | | | | 57 | |

Experiment B animals treated with 4 doses of AGLU (1.0, 2.0, 1.0 and 1.4 mg) 6 days apart

| | | | | | | | | | | | | | | |
|---|---|---|---|---|---|---|---|---|---|---|---|---|---|---|
| treated KO | 1132 | 70 | — | — | 24 | 1259 | 125 | 87 | — | — | 89 | — | 0.4 | 163 |
| mice (13 weeks | 944 | 13 | | | 10 | 1082 | 46 | 116 | | | 35 | | 0.2 | 163 |
| old) | | | | | | | | | | | | | | |
| treated KO | 3375 | 23 | — | — | 60 | 1971 | 49 | 90 | — | — | 207 | — | 0.7 | 374 |
| mice (34 weeks | | | | | | | | | | | | | | |
| old) | | | | | | | | | | | | | | |
| untreated KO | 2.0 | 406 | — | — | 0.2 | 3233 | 1.0 | 86 | — | — | 1.0 | — | 0.2 | 487 |
| mice (13 and | 2.0 | 147 | | | 0.3 | 1748 | 1.0 | 87 | | | 1.0 | | 0.2 | 168 |
| 34 weeks old) | | | | | | | | | | | | | | |
| untreated | 35 | 6 | — | — | 8.2 | 0 | 6.0 | 1.0 | — | — | 14 | — | 18 | 0 |
| normal mice | | | | | | | | | | | | | | |
| (34 weeks old) | | | | | | | | | | | | | | |

Experiment C animals treated with single dose of 0.7 mg

| | | | | | | | | | | | | | | |
|---|---|---|---|---|---|---|---|---|---|---|---|---|---|---|
| treated KO | 582 | — | 462 | — | 46 | — | — | — | 5.1 | — | — | — | 0.4 | — |
| mice | 558 | | 313 | | 50 | | | | 3.6 | | | | 0.4 | |
| untreated KO | 1.1 | — | 0.8 | — | 0.3 | — | — | — | 0.2 | — | — | — | 0.2 | — |
| mice | 1.6 | | 0.7 | | 0.3 | | | | 0.3 | | | | 0.2 | |

Figures in the table refer to individual animals
Act: AGLU activity (nmoles 4 MU per mg protein per hour)
Glc: Glycogen content (μg/mg protein)
n.d. not detected
— data unavailable

When two KO mice were injected 4 times every 6 days (experiment B), a marked decrease of total cellular glycogen was observed in both heart and liver. No effects were observed in skeletal muscle tissues with regard to total glycogen. However, in general the uptake of AGLU in these tissues was lower than in the other tissues tested. Transmission electron microscopy of the 4 times injected KO mice indicated a marked decrease in lysosomal glycogen in both liver cells and heart muscle cells. The effects observed in heart tissue are localized since in some areas of the heart no decrease in lysosomal glycogen was observed after these short term administrations.

Western blot analysis using rabbit polyclonal antibodies against human placenta mature alpha-glucosidase indicated complete processing of the injected AGLU towards the mature enzyme in all organs tested strongly suggesting uptake in target tissues, and lysosomal localization and processing. No toxic effects were observed in any of the three experiments. Immunohistochemical staining of AGLU was evident in lysosomes of hepatocytes using a polyclonal rabbit antibody against human alpha-glucosidase. The presence of AGLU in heart and skeletal tissues is more difficult to visualize with this technique, apparently due to the lower uptake.

2. Long-term Experiments with the KO Mouse Model

In longer term experiments, enzyme was injected in the tail vein of groups of two or three KO mice, once a week for periods of up to 25 weeks. The initial dose was 2 mg (68 mg/kg) followed by 0.5 mg (17 mg/kg)/mouse for 12 weeks. In two groups of mice, this was followed by either 4 or 11 additional weeks of treatment of 2 mg/mouse. Injections started when the mice were 6-7 months of age. At this age, clear histopathology has developed in the KO model. Two days after the last enzyme administration the animals were killed, and the organs were perfused with phosphate buffered saline (PBS). Tissue homogenates were made for AGLU enzyme activity assays and tissue glycogen content, and sections of various organs were made to visualize (via light microscopy) lysosomal glycogen accumulation.

The results showed that mice treated 13 weeks with 0.5 mg/mouse (Group A, 3 animals/Group) had an increase of activity in the liver and spleen and decreased levels of glycogen in liver and perhaps in heart. One animal showed increased activity in muscles, although there was no significant decrease of glycogen in muscle.

Mice that were treated 14 weeks with 0.5 mg/mouse followed by 4 weeks with 2 mg/mouse (Group B, 3 animals/Group) showed similar results to those treated for 13 weeks only, except that an increased activity was measured in the heart and skeletal muscles and decreases of glycogen levels were also seen in the spleen.

Mice that were treated 14 weeks with 0.5 mg/mouse followed by 11 weeks with 2 mg/mouse (Group C 2 animals/Group) showed similar results to the other two groups except that treated mice showed definite decreases in glycogen levels in liver, spleen, heart and skeletal muscle. No activity could be detected, even at the highest dose, in the brain.

Results of treated and untreated animals in each Group (Group means) are summarized in Table 3.

TABLE 3
Tissue Uptake of AGLU and Glycogen Content Following Long Term Treatment in KO Mouse Model

| | | | | | | | Pectoral | | Quadriceps | | Gastroenemius | | | |
|---|---|---|---|---|---|---|---|---|---|---|---|---|---|---|
| | Liver | | Spleen | | Heart | | Muscle | | Muscle | | Muscle | | Brain | |
| Group | Act | Glc | Act | Glc | Act | Glc | Act | Glc | Act | Glc | Act | Glc | Act | Glc |

Group A animals treated with 0.5 mg/mouse/week for 13 weeks

| | | | | | | | | | | | | | | |
|---|---|---|---|---|---|---|---|---|---|---|---|---|---|---|
| treated | 713 | 2 | 463 | n.d. | 3 | 86 | 9 | 81 | 6 | 40 | 14 | 66 | — | — |
| untreated | 2 | 24 | 1 | n.d. | 1 | 111 | 1 | 66 | 1 | 50 | 1 | 61 | — | — |

Group B animals treated with 0.5 mg/mouse/week for 14 weeks, followed by 2 mg/mouse./week for 4 weeks

| | | | | | | | | | | | | | | |
|---|---|---|---|---|---|---|---|---|---|---|---|---|---|---|
| treated | 2705 | 1 | 1628 | 0 | 59 | 288 | 49 | 120 | 30 | 128 | 44 | 132 | — | — |
| untreated | 3 | 11 | 31 | 6 | 1 | 472 | 1 | 113 | 1 | 162 | 1 | 142 | — | — |

Group C animals treated with 0.5 mg/mouse/week for 14 weeks, followed by 2 mg/mouse./week for 11 weeks

| | | | | | | | | | | | | | | |
|---|---|---|---|---|---|---|---|---|---|---|---|---|---|---|
| treated | 1762 | 1 | 1073 | 2 | 66 | 211 | 99 | 113 | 37 | 18 | 109 | 32 | 1 | 32 |
| untreated | 2 | 45 | 1 | 21 | 1 | 729 | 1 | 291 | 0 | 104 | 0 | 224 | 0 | 44 |

Figures in the table refer to the mean of 3 animals (Groups A and B) or the mean of 2 animals (Group C)
Act AGLU activity (nmoles 4 MU per mg protein per hour)
Glc: Glycogen content (μg/mg protein)
n.d. not detected
— data unavailable

In addition, a very convincing improvement in the histopathological condition was observed in Group C mice (treated for the first 14 weeks at 0.5 mg/mouse, followed by 11 weeks at 2 mg/mouse). Clear reversal of pathology was demonstrated in various tissues, such as heart and pectoralis muscle.

It has been reported that Pompe's disease does not occur when the residual lysosomal a-glucosidase activity is >20% of average control value (14). The data obtained with the KO mouse model indicates that these levels are very well achievable using recombinant precursor enzyme.

### Example 5

### Human Clinical Trial

A single phase I study (AGLU1101-01) has been conducted in 15 healthy male volunteers. Doses of AGLU ranged from 25 to 800 mg, administered by intravenous infusion to healthy male adult volunteers. Subjects with a history of allergies and hypersensitivities were excluded from the study. The subjects were randomized into dose groups of 5, and each dose Group received AGLU (4 subjects) or placebo (1 subject) at each dose level. All subjects received two doses of study drug, which were administered two weeks apart. The dosing regimen was as follows:

A

25 mg: Group 1, treatment period 1

B

50 mg: Group 1, treatment period 2

C

100 mg: Group 2, treatment period 1

D

200 mg: Group 3, treatment period 1

E

400 mg: Group 2, treatment period 2

F

800 mg: Group 3, treatment period 2

placebo (1 subject per Group and treatment period)

Subjects were administered AGLU or placebo as an infusion on Day 1 of each treatment period. The infusions were administered over a 30 minute period and subjects were kept in a semi-recumbent position for at least 2 hours after cessation of infusion.

Adverse events were recorded just before the start of the infusion, at 30 minutes (end of infusion) and at 3, 12, 24, 36 and 48 hours thereafter as well as on Days 5 and 8 (first period) and days 5, 8 and 15 (second period). Vital signs, ECG and physical examinations were also monitored regularly throughout the treatment period.

Blood samples were taken for a standard range of clinical laboratory tests and pharmacokinetics analysis. The subject's urine was collected and a standard range of laboratory analyses (including determination of AGLU) were performed.

(a) Laboratory Safety and Adverse Events

There were no clinically significant changes in laboratory parameters, clinical signs and ECG measurements in any subjects at any dose Group. The results of adverse event monitoring in all subjects at all doses are summarized in Table 4.

TABLE 4
Adverse Event Reports

| Dose (mg) | Adverse Events |
|---|---|
| 25 | The reported events were muscle weakness, abnormal vision and fatigue. All events were |
| | mild and were deemed unrelated to the test article by the investigator. |
| 50 | The reported events were headache, rhinitis, nose bleed and paresthesia. All events were |
| | mild and were deemed unrelated or remotely related to the test article by the investigator, |
| | except the paresthesia which was classed as moderate and was deemed possibly related to the |
| | test article. |
| 100 | The reported events were rhinitis, headache, fatigue, hematoma and injection site reaction. |
| | All events were classed as mild. The cases of hematoma, injection site reaction and |
| | intermittent headache were deemed possibly or probably related to the test article by the |
| | investigator. The other events were deemed to be unrelated. |
| 200 | The reported events were nausea, headache, dizziness, fatigue, rhinitis, photophobia, vision |
| | abnormalities and euphoria. All events were classed as mild or moderate in intensity. Seven |
| | events (including cases of dizziness, nausea and abnormal vision) were deemed to be |
| | possibly or probably related to the test article. |
| 400 | The reported events were fatigue and paresthesia. The report of fatigue was considered |
| | unrelated to the test article, and the paresthesia was deemed possibly related. |
| 800 | The reported events were nausea, drowsiness, dizziness, increased sweating, asthenia, |
| | shivering and intermittent headache. All events were classed as mild or moderate in intensity. |
| | Eight events (including cases of drowsiness, nausea and asthenia) were deemed to be |
| | possibly related to the test article. |

A trial of the safety and efficacy of recombinant acid a-glucosidase as enzyme replacement therapy on infantile and juvenile patients with glycogen storage disease Type II is conducted. Four infantile patients and three juvenile patients are recruited. Infantiles are administered a starting dose of 15-20 mg/kg titrated to 40 mg/kg and juveniles are administered 10 mg/kg. Patients are treated for 24 weeks.

Patients are evaluated by the following parameters:

Standard adverse event reporting including suspected adverse events

Laboratory parameters including hematology, clinical chemistry and antibody detection.

a-glucosidase activity in muscle

Muscle histopathology

12-lead ECO

Clinical condition including neurological examination

Non-parametric PK parameters

Life saving interventions

Infantile patients are evaluated for the following additional parameters.

Left posterior ventricular wall thickness and left ventricular mass index

Neuromotor Development

Survival

Glycogen content in muscle

Juvenile patients are evaluated for the following additional parameters.

Pulmonary function

Muscle strength/timed tests and muscle function

PEDI/Rotterdam 9-item scale

The same patients are then subject to additional dosages of alpha glucosidase with infantiles receiving 15, 20, 30 or 40 mg/kg and juveniles: 10 mg/kg for an additional period of 24 weeks and evaluated by the parameters indicated above.

A further phase II clinical trial is performed on eight patients aged <6 months of age within 2 months after diagnosis at a dosage of 40 mg/kg. Patients are treated for 24 weeks and evaluated by the following criteria:

Safety parameters

Laboratory safety data

Adverse event recording

Primary efficacy parameter: survival without life-saving interventions (i.e. mechanical ventilation>24 hr) 6 months past diagnosis in combination with normal or mildly delayed motor function (BSID II).

Secondary efficacy: Changes in neuromotor development; changes in left posterior ventricular wall thickness and left ventricular mass index; Changes in skeletal muscle acid a-glucosidase activity and glycogen content.

Efficacy can be shown by a 50% survival at 6 months post-diagnosis without life saving interventions in the a-glucosidase group compared to 10% survival in the historical control group in combination with a BSID II classified as normal or mildly delayed.

A further clinical trial is performed on juvenile patients. The patients are aged >1 year and <35 years of age with juvenile onset of GSD type IIb The patients are administered 10 mg/kg or 20 mg/kg for a period of twenty-four weeks treatment.

Treatment is monitored by the following parameters:

Safety parameters Laboratory safety data

Adverse event recording

Primary efficacy Pulmonary function parameters (e.g. FVC, time on ventilator)

Muscle strength

Secondary efficacy Life-saving interventions parameters:

Quality of life

Skeletal muscle acid a-glucosidase activity

Quantitative objective 20% relative improvement in primary efficacy parameters over baseline

All quantitative measurements relating to efficacy are preferably statistically significant relative to contemporaneous or historical controls, preferably at p<0.05.

### Example 6

### Pharmaceutical Formulations

Alpha-glucosidase is formulated as follows: 5 mg/ml Cl-Glu, 15 mM sodium phosphate, pH 6.5,2% (w/w) mannitol, and 0.5% (w/w) sucrose. The above formulation is filled to a final volume of 10.5 ml into a 20 cc tubing vial and lyophilized. For testing, release and clinical use, each vial is reconstituted with 10.3 ml* of sterile saline (0.9%) for injection (USP or equivalent.) to yield 10.5 ml of a 5 mg/ml 0 Glu solution that may be directly administered or subsequently diluted with sterile saline to a patient specific target dose concentration. The 10.5 ml fill (52.5 mg alpha glucosidase total in vial) includes the USP recommended overage, that allows extraction and delivery (or transfer) of 10 mls (50 mg). The mannitol serves as a suitable bulking agent shortening the lyophilization cycle (relative to sucrose alone). The sucrose serves as a cryo/lyoprotectant resulting in no significant increase in aggregation following reconstitution. Reconstitution of the mannitol (only) formulations had repeatedly resulted in a slight increase in aggregation. Following lyophilization, the cake quality was acceptable and subsequent reconstitution times were significantly reduced Saline is preferred to HSA/dextrose for infusion solution. When saline is used in combination with lyophilization in 2% mannitol/0.5% sucrose the solution has acceptable tonicity for intravenous administration. The lyophilized vials containing the 2% mannitol/0.5% sucrose formulation were reconstituted with 0.9% sterile saline (for injection) to yield 5 mg/ml 0-Glu.

### Example 7

### Infusion Schedule

The solution is administered via the indwelling intravenous cannula. Patients are monitored closely during the infusion period and appropriate clinical intervention are taken in the event of an adverse event or suspected adverse event. A window of 48 hours is allowed for each infusion. An infusion schedule in which the rate of infusion increases with time reduces or eliminates adverse events.

Infusions for infantiles can be administered according to the following schedule: 5 cc/hr for 60 minutes 0 10 cc/hr for 60 minutes * 2 40 cc/hr for 30 minutes 280 cc/hr for the remainder of the infusion.

Infusions for juveniles can be administered according to the following schedule:

0.5 cc/kg/hr for 60 minutes

1 cc/kg/hr for 60 minutes

5 cc/kg/hr for 30 minutes

7.5 cc/kg hr for the remainder of the infusion

While the foregoing invention has been described in some detail for purposes of clarity and understanding, it will be clear to one skilled in the art from a reading of this disclosure that various changes in form and detail can be made without departing from the true scope of the invention. All publications and patent documents cited in this application are incorporated by reference in their entirety for all purposes to the same extent as if each individual publication or patent document were so individually denoted.

What is claimed is:

1. A transgenic nonhuman mammal having a transgene comprising:
a promoter and enhancer from the same mammary gland specific gene;
a secretory DNA segment encoding a signal peptide functional in mammary secretory cells of the transgenic nonhuman mammal, and
a recombinant DNA segment encoding acid .alpha.-glucosidase operably linked to the secretory DNA segment to form a secretory-recombinant DNA segment, the secretory-recombinant DNA segment being operably linked to the promoter and enhancer, and wherein the secretory DNA segment is an acid a glucosidase secretory DNA segment or is from the same mammary-gland specific gene as the promoter and enhancer;
wherein the transgene, in an adult form of the nonhuman mammal or a female descendant of the nonhuman mammal, expresses the secretory-recombinant DNA segment in the mammary secretory cells to produce acid .alpha.-glucosidase that is processed and secreted by the mammary secretory cells into milk in a recoverable amount with .alpha.-glucosidase catalytic activity.

2. The transgenic nonhuman mammal of claim 1, wherein the concentration of the acid .alpha.-glucosidase in the milk is at least 100 .mu.g/ml.

3. The nonhuman transgenic mammal of claim 1, wherein the secretory DNA segment is an acid .alpha.-glucosidase secretory DNA segment.

4. The transgenic nonhuman mammal of claim 1, wherein the human acid .alpha.-glucosidase is secreted into milk in a form that can be taken up by muscle cells.

5. The nonhuman transgenic mammal of claim 1, wherein the acid .alpha.-glucosidase is human.

6. The nonhuman transgenic mammal of claim 5, that is a mouse or rabbit.

7. The nonhuman transgenic mammal of claim 6, wherein the recombinant DNA segment is cDNA.

8. The nonhuman transgenic mammal of claim 6, wherein the recombinant DNA segment is genomic.

9. The nonhuman transgenic mammal of claim 6, wherein the recombinant DNA segment is a cDNA-genomic-DNA hybrid.

10. A method for producing acid .alpha.-glucosidase, the method comprising:
recovering milk from the adult form of the transgenic nonhuman mammal of claim 1 or its female descendant, wherein said milk contains a recoverable amount of acid .alpha.-glucosidase with catalytic activity.

11. The method of claim 10, further comprising incorporating the milk into a food product.

12. The method of claim 10, further comprising purifying the acid .alpha.-glucosidase from the milk.

13. The method of claim 12, wherein the acid .alpha.-glucosidase is purified to at least 95% pure from other components of the milk.

14. The method of claim 13, further comprising mixing the acid .alpha.-glucosidase with a pharmaceutical carrier for intravenous, intradermal, intramuscular or oral administration.

15. Milk from the transgenic nonhuman mammal of claim 1, the milk comprising human acid .alpha.-glucosidase in a recoverable amount.

16. The milk of claim 15, wherein the concentration of the human acid .alpha.-glucosidase is at least 100 .mu.g/ml.

17. A composition comprising human acid .alpha.-glucosidase with catalytic activity and capacity to be taken up by muscle cells in a patient and milk of the nonhuman transgenic mammal of claim 1.

18. A pharmaceutical composition for parenteral administration to a human patient comprising human acid .alpha.-glucosidase with catalytic activity and in a therapeutically effective dosage to treat a patient suffering from Pompe's disease; and a pharmaceutical carrier, the composition being free of other human proteins present in its natural environment.

19. The pharmaceutical composition of claim 18, wherein the pharmaceutical carrier is for intravenous administration.

20. The pharmaceutical composition of claim 18, wherein the human acid .alpha.-glucosidase is purified to homogeneity.

21. A method of treating a patient with Pompe's disease, comprising:
administering to the patient a therapeutically effective amount of human acid alpha glucosidase.

22. The method of claim 21, wherein the patient is administered at least 10 mg/kg body weight per week.

23. The method of claim 21, wherein the patient is administered at least 15 mg/kg body weight per week.

24. The method of claim 21, wherein the patient is administered at least 20 mg/kg body weight per week.

25. The method of claim 21, wherein the patient is administered at least 30 mg/kg body weight per week.

26. The method of claim 21, wherein the patient is administered at least 45 mg/kg-60 mg/kg body weight per week.

27. The method of claim 21, wherein the patient is administered at least 60 mg/kg body weight per week.

28. The method of claim 21, wherein the patient is administered at least 120 mg/kg body weight per week.

29. The method of any of claims 21-28, wherein the patient is administered a single dosage of alpha-glucosidase per week.

30. The method of any of claims 21-28, wherein the patient is administered two dosages of alpha-glucosidase per week.

31. The method of any of claim 21-28, wherein the patient is administered three dosages of alpha-glucosidase per week.

32. The method of any of claims 21-31, wherein the amount is administered per week for a period of at least four weeks.

33. The method of any of claims 21-31, wherein the amount is administered per week for a period of at least 24 weeks.

34. The method of any of claim 21-33, wherein the alpha-glucosidase is administered intravenously.

35. The method of claim 21, wherein the alpha-glucosidase was produced in milk of a transgenic mammal.

36. The method of claim 21, wherein the alpha-glucosidase was produced from a CHO cell-line.

37. The method of claim 21, wherein the patient has infantile Pompe's disease.

38. The method of claim 21, wherein the patient survives to be at least one year old.

39. The method of claim 21, wherein the patient has juvenile Pompe's disease.

40. The method of claim 21, wherein the patient has adult Pompe's disease.

41. The method of claim 21, wherein the alpha-glucosidase is predominantly in a 110 kD form.

42. The method of claim 21, further comprising monitoring a level of human acid alpha glucosidase in the patient.

43. The method of claim 21, further comprising administering a second dosage of human acid alpha glucosidase if the level of alpha-glucosidase falls below a threshold value in the patient.

44. The method of claim 21, wherein the human alpha glucosidase is administered intravenously and the rate of administration increases during the period of administration.

45. The method of claim 44, wherein the rate of administration increases by at least a factor of ten during the period of administration.

46. The method of claim 44, wherein the rate of administration increases by at least a factor of ten within a period of five hours.

47. The method of claim 21, wherein the patient is administered a series of at least four dosages, each dosage at a higher strength than the previous dosage.

48. The method of claim 47, wherein the dosages are a first dosage of 0.03-3 mg/kg/hr, a second dosage of 0.3-12 mg/kg/hr, a third dosage of 1-30 mg/kg/hr and a fourth dosage of 2-60 mg/kg/hr.

49. The method of claim 47, wherein the dosages are a first dosage of 0.11 mg/kg/hr, a second dosage of 1-4 mg/kg/hr, a third dosage of 3-10 mg/kg/hr and a fourth dosage of 6-20 mg/kg/hr.

50. The method of claim 47, wherein the dosages are a first dosage of 0.25-4 mg/kg/hr, a second dosage of 0.9-1.4 mg/kg/hr, a third dosage of 3.6-5.7 mg/kg/hr and a fourth dosage of 7.2-11.3 mg/kg/hr.

51. The method of claim 23, wherein the dosages are a first dosage of 0.3 mg/kg/hr, a second dosage of 1 mg/kg/hr, a third dosage of 4 mg/kg/hr and a fourth dosage of 12 mg/kg/hr

52. The method of any of claims 47-51, wherein the first, second, third and fourth dosages are each administered for periods of 15 min to 8 hours.

53. The method of any of claims 47-51, wherein the first, second, third and fourth dosages are administered for periods of 1 hr, 1 hr, 0.5 hr and 3 hr respectively.

54. A pharmaceutical composition comprising human acid alpha.glucosidase, human serum albumin, and a sugar in a physiologically acceptable buffer in sterile form.

55. The pharmaceutical composition of claim 54 comprising human acid alpha glucosidase, human serum albumin, and glucose in sodium phosphate buffer.

56. A pharmaceutical composition comprising alpha glucosidase, mannitol and sucrose in an aqueous solution.

57. The pharmaceutical composition of claim 56, wherein the sugar comprises mannitol and sucrose and the concentration of mannitol is 1-3% w/w of the aqueous solution and the concentration of sucrose is 0.1 to 1% w/w of the aqueous solution.

58. The pharmaceutical composition of claim 56, wherein the concentration of mannitol is 2% w/w and the concentration of sucrose is 0.5% w/w.

59. A lyophilized composition produced by lyophilizing a pharmaceutical composition comprising human acid glucosidase, mannitol and sucrose in aqueous solution.

60. A pharmaceutical composition prepared by lyophilizing a first composition comprising human acid alphaglucosidase, mannitol, sucrose and an aqueous solution to produce a second composition; and reconstituting the lyophilized composition in saline to produce a third composition.

61. The pharmaceutical composition of claim 60, wherein the human acid alpha-glucosidase is at 5 mg/ml in both the first and third composition, the mannitol is at 2 mg/ml in the first composition, the sucrose is at 0.5 mg/ml in the first composition, and the saline used in the reconstituting step is 0.9% w/w.
